SEQUENCE LISTING (460 sequences)

SEQ ID NO: 1 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSTISGSGGSTDYADSVKGRFTISRDNSKNTLYLQMNSLRAEDTAVYYCADGRGPFFDYRGQGTLVTVSS

SEQ ID NO: 2 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSTISGGGGSTDYADSVKGRFTISRDNSKNTLYLQLNSLRAEDTAVYYCADGRGPFFDYRGQGTLVTVSS

SEQ ID NO: 3 - protein, 117 aa, Artificial Sequence
  source: /note=“Description of Artificial Sequence: Synthetic polypeptide”
EVQVLESGGGLVQPGDSLRLSCAASGFTFTNFAMSWVRQAPGKGLEWVSTISGSGGSTDYADSVKGRFTISRDNSKNMLYLQMNSLRAEDTAVYYCADGRGPFFDYRGQGTLVTVSS

SEQ ID NO: 4 - protein, 117 aa, Artificial Sequence
  source: /note=“Description of Artificial Sequence: Synthetic polypeptide”
EVQVLESGGGLVQPGGSLRLSCEASGFTFSNFAMSWVRLAPGKGLEWVSTISGSGGSTSYADSVKGRFTISRDNSKNTLYLQMNSLRAEDTAVYYCADGRGPFFDYRGQGTLVTVSS

SEQ ID NO: 5 - protein, 117 aa, Artificial Sequence
  source: /note=“Description of Artificial Sequence: Synthetic polypeptide”
EVQVLESGGGLVQPGGSLRLSCATSGFTFSNFAMSWVRQAPGKGLEWVSTISGSGGSTDYADSVKGRFTISRDNSKNTLHLQMNSLRAEDTAVYYCADGRGPFFDYRGQGTLVTVSS

SEQ ID NO: 6 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSTISGSGGSTDYADSVKGRFTISRDNSKNTLYLQMNSLRAEDTAVYYCADGRGPFFDYRGQGTLVTVSS

SEQ ID NO: 7 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSTISGSGDSTDYADSVKGRFTISRDNSKKTLYLQMNSLRVEDTAVYYCADGRGPFFDYRGQGTLVTVSS

SEQ ID NO: 8 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSAISGSGGSTDYADSVKGRFTISRDNSNNTLYLQMNSLRAEDTAVYYCADGTGPFFDYRGQGTLVTVSS

SEQ ID NO: 9 - protein, 117 aa, Artificial Sequence
  source: /note=“Description of Artificial Sequence: Synthetic polypeptide”
EVQVLESGGGLVQPGGSLRLSCEASGFTFSNFAMSWVRQAPGKGLEWVSTISGSGGTTDYADSVKGRFTISRDNSKKTLYLQMNSLRVEDTAVYYCADGRGPFFDYRGQGTLVTVSS

SEQ ID NO: 10 - protein, 117 aa, Artificial Sequence
  source: /note=“Description of Artificial Sequence: Synthetic polypeptide”
EVQVLESGGGLVQPGESLRLSCAVSGFTFSNFAMSWVRQAPGKGLEWVSGISGSGGSTDYADSVKGRFTISRDNSKNTLYLQMNSLRAEDTALYYCADGTGPFFDYRGQGTLVTVSS

SEQ ID NO: 11 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSGISGSGGTTDYADSVKGRFTISRDNSKNTLYLQMNSLRVEDTAVYYCADGTGPFFDYRGQGTLVTVSS

SEQ ID NO: 12 - protein, 117 aa, Artificial Sequence
  source: /note=“Description of Artificial Sequence: Synthetic polypeptide”
EVQVLESGGGLVQPGGSLRLSCAAAGFTFSNFAMSWVRQAPGKGLEWVSSISGSAGTIDYADSVKGRFTISRDNSKNTLYLQMNSLRAEDTAVYYCADGTGPFFDYRGQGTLVTVSS

SEQ ID NO: 13 - protein, 117 aa, Artificial Sequence
  source: /note=“Description of Artificial Sequence: Synthetic polypeptide”
EVQVLESGGGLVQPGGSLRLSCAASGFTFSRYAMSWVRQAPGKGLEWVSAISGSGDSTDYADSVKGRFTISRDNSKNTLYLQMNSLRAEDTAVYYCADGTGPFFDYRGQGTLVTVSS

SEQ ID NO: 14 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSTISGSGGSTDYADSVKGRFTISRDNSKNTLYLQMNSLRVEDTAVYYCADGTGPFFDYRGQGTLVTVSS

SEQ ID NO: 15 - protein, 117 aa, Artificial Sequence
  source: /note=“Description of Artificial Sequence: Synthetic polypeptide”
EVQVLESGGGLVQPGESLRLSCAVSGFTFSNFAMSWVRQAPGKGLEWVSGISGSGGSTDYADSVKGRFTISRDNSKNTLYLQMNSLRAEDTAVYYCADGTGPFFDYRGQGTLVTVSS

SEQ ID NO: 16 - protein, 117 aa, Artificial Sequence
  source: /note=“Description of Artificial Sequence: Synthetic polypeptide”
EVQVLESGGGLVQPGGSLRLSCATSGFTFSNYAMSWVRQAPGKGLEWVSTISGSGGNTDYADSVKGRFTISRDNSKNTLYLQMNSLRAEDTAVYYCADGTGPFFDYRGQGTLVTVSS

SEQ ID NO: 17 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSGISNSGGSTDYADSVKGRFTISRDNSKNTLYLQMNSLRVEDTAVYYCADGTGPFFDYRGQGTLVTVSS

SEQ ID NO: 18 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSAISGSGGSTDYADSVKGRFTISRDNSKNTLYLQMNSLRAEDTAVYYCADGTGPFFDYRGQGTLVTVSS

SEQ ID NO: 19 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSGISGSGGSTDYADSVKGRFTISRDNSKNTLYLQMNSLRAEDTAVYYCADGTGPFFDYRGQGTLVTVSS

SEQ ID NO: 20 - protein, 117 aa, Artificial Sequence
  source: /note=“Description of Artificial Sequence: Synthetic polypeptide”
EVQVLESGGGLVQPGGSLRLSCAASGFTFSNYAMSWVRQAPGKGLEWVSAISGSGGSTYYADSVKGRFTISRDNSKNTLHLQMNSLRAEDTAVYYCADGRGPFFDYRGQGTLVTVSS

SEQ ID NO: 21 - protein, 117 aa, Artificial Sequence
  source: /note=“Description of Artificial Sequence: Synthetic polypeptide”
EVQVLESGGGLVQPGGSLRLSCAASGFTFSSFAMSWVRQAPGKGLEWVSAISGSGGTTDYADSVKGRFTISRDNSMNTLYLQMNSLRAEDTAVYYCADGTGPFFDYRGQGTLVTVSS

SEQ ID NO: 22 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCADGRGPFFDYRGQGTLVTVSS

SEQ ID NO: 23 - protein, 117 aa, Artificial Sequence
  source: /note=“Description of Artificial Sequence: Synthetic polypeptide”
EVQVLESGGGLVQPGGSLRLSCAASGFTFSSYAMNWVRQAPGKGLEWVSAISGSAGRTDYADSVKGRFTISRDNSKNTLYLQMNSLRAEDTAVYYCADGTGPFFDYRGQGTLVTVSS

SEQ ID NO: 24 - protein, 117 aa, Artificial Sequence
  source: /note=“Description of Artificial Sequence: Synthetic polypeptide”
EVQVLESGGGVVQPGRSLRLSCAASGFTFSSYAMSWVRQAPGKGLEWVSTISGGGGNTDYADSVKGRFTISRDNSNNMLYLQMNSLRAEDTAVYYCADGTGPFFDYRGQGTLVTVSS

SEQ ID NO: 25 - protein, 117 aa, Artificial Sequence
  source: /note=“Description of Artificial Sequence: Synthetic polypeptide”
EVQVLESGGGLVQPGGSLRLSCAASGFTFSSYAMSWVRQGPGKGLEWVSTISGSGDSTDYADSVKGRFTISRDNSKNTLYLQMNSLRTEDTAVYYCADGTGPFFDYRGQGTLVTVSS

SEQ ID NO: 26 - protein, 117 aa, Artificial Sequence
  source: /note=“Description of Artificial Sequence: Synthetic polypeptide”
EVQVLESGGGLVQPGGSLRLSCAAAGFTFSNFAMSWVRQAPGKGLEWVSTISGGGGGTDYADSVEGRFTISRDNSKNTLYLQMNSLRAEDTAVYYCADGRGPFFDYRGQGTLVTVSS

SEQ ID NO: 27 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSTISGSGGSTDYADSVKGRFTISRDNSKNMLYLQMNSLRAEDTAVYYCADGTGPFFDYRGQGTLVTVSS

SEQ ID NO: 28 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADSVKGRFTISRDNSKNTLYLQMNSLRVEDTAVYYCADGRGPFFDYRGQGTLVTVSS

SEQ ID NO: 29 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSGISNSGGSTDYADSVKGRFTISRDNSKNTLYLQMDSLRAEDTAVYYCADGTGPFFDYRGQGTLVTVSS

SEQ ID NO: 30 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADSVKGRFTISRDNSKNTLYLQMNSLRAEDTAVYYCADGTGPFFDYRGQGTLVTVSS

SEQ ID NO: 31 - protein, 117 aa, Artificial Sequence
  source: /note=“Description of Artificial Sequence: Synthetic polypeptide”
EVQVLESGGGLVQPGGSLRLSCAASGFTFSNYAMSWVRQAPGKGLEWVSSISGSAGSIDYADSVRGRFTISRDNSNNTLYLQMNSLRAEDTAVYYCADGTGPFFDYRGQGTLVTVSS

SEQ ID NO: 32 - protein, 117 aa, Artificial Sequence
  source: /note=“Description of Artificial Sequence: Synthetic polypeptide”
EVQVLESGGGLVQPGGSLRLSCAASGFTFSNYAMSWVRQAPGKGLEWVSTISGSGGSTDYADSVKGRFTISRDNSKNTLYLQLNSLRAEDTAVYYCADGTGPFFDYRGQGTLVTVSS

SEQ ID NO: 33 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVAVISYDGSNKYYADSVKGRFTISRDNSKNTLYLQMNSLRAEDTAVYYCADGTGPFFDYRGQGTLVTVSS

SEQ ID NO: 34 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGSNTYYADSVKGRFTISRDNSKNTLYLQMNSLRAEDTAVYYCADGRGPFFDYRGQGTLVTVSS

SEQ ID NO: 35 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSGISNRGVNTYYADNVKGRFIISRDNSKNTLYLQLNSLRAEDTAVYYCADGTGPFFDYRGQGTLVTVSS

SEQ ID NO: 36 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTDYADSVKGRFTISRDNSKNTLYLQMNSLRAEDTAVYYCADGTGPFFDYRGQGTLVTVSS

SEQ ID NO: 37 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSAISGSGGSTYYADSVKGRFTISRDNSKNTLYLQMNSLRVEDTAVYYCADGTGPFFDYRGQGTLVTVSS

SEQ ID NO: 38 - protein, 117 aa, Artificial Sequence
  source: /note=“Description of Artificial Sequence: Synthetic polypeptide”
EVQVLESGGGLVQPGGSLRLSCAASGFTFSNFAMSWVRQAPGKGLEWVSTISGSGDSTYYADSVKGRFTISRDNSRNTVYLQMNSLRAEDTAVYYCADGTGPFFDYRGQGTLVTVSS

SEQ ID NO: 39 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADSVKGRFTISRDNSKNTLHLQMNSLRAEDTAVYYCADGRGPFFDYRGQGTLVTVSS

SEQ ID NO: 40 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADSVKGRFTISRDNSKNTLYLQMNSLRVEDTAVYYCADGTGPFFDYRGQGTLVTVSS

SEQ ID NO: 41 - protein, 117 aa, Artificial Sequence
  source: /note=“Description of Artificial Sequence: Synthetic polypeptide”
EVQVLESGGGLVQPGGSLRLSCAASGFTFSSYGMHWVRQAPGKEREWVAVISYDGGNKYYADSVKGRFTISRDNSNNTLYLQMNSLRTEDTAVYYCADGTGPFFDYRGQGTLVTVSS

SEQ ID NO: 42 - protein, 117 aa, Artificial Sequence
  source: /note=“Description of Artificial Sequence: Synthetic polypeptide”
EVQVLESGGGLVQPGGSLRLSCAASGFIFSSYAMSWVRQAPGKGLEWVSTISGSGGSTDYADSVKGRFTISRDNSKNTLHLQMNSLRAEDTAVYYCADGTGPFFDYRGQGTLVTVSS

SEQ ID NO: 43 - protein, 117 aa, Artificial Sequence
  source: /note=“Description of Artificial Sequence: Synthetic polypeptide”
EVQVLESGGGLVQPGGSLRLSCVASGFTFSGYGMHWARQAPGKEREWVAVISYDGSKKYYADSVKGRFTISRDNSKNTLYLQMNSLRAEDTAVYYCADGRGPFFDYRGQGTLVTVSS

SEQ ID NO: 44 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADSVKGRFTISRDNSNNTLYLQMNSLRAEDTAVYYCADGTGPFFDYRGQGTLVTVSS

SEQ ID NO: 45 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSAGTIDYADSVKGRFTISRDNSKNTLYLQMNSLRAEDTAVYYCADGTGPFFDYRGQGTLVTVSS

SEQ ID NO: 46 - protein, 117 aa, Artificial Sequence
  source: /note=“Description of Artificial Sequence: Synthetic polypeptide”
EVQVLESGGGLVQPGGSLRLSCAAAGFTFSSYAMSWVRQAPGKGLEWVSVISGSGGSTNYADSVKGRFTISRDNSKNTLYLQMNSLRAEDTAVYYCADGRGPFFDYRGQGTLVTVSS

SEQ ID NO: 47 - protein, 117 aa, Artificial Sequence
  source: /note=“Description of Artificial Sequence: Synthetic polypeptide”
EVQVLESGGGLVQPGGSLRISCAASGFTFSSFAMSWVRQAPGKGREWVAVISYDGSNKYYADSVKGRFTISRDNSKNTLYLQMNSLRAEDTAVYYCADGTGPFFDYRGQGTLVTVSS

SEQ ID NO: 48 - protein, 117 aa, Artificial Sequence
  source: /note=“Description of Artificial Sequence: Synthetic polypeptide”
EVQVLESGGGLVQPGGSLRLSCAASGFTFSSYAMSWVRQAPGKEREWLAVISYDGSKKYYADSVKGRFTISRDNSKNTLYLQMNSLRAEDTAVYYCADGTGPFFDYRGQGTLVTVSS

SEQ ID NO: 49 - protein, 117 aa, Artificial Sequence
  source: /note=“Description of Artificial Sequence: Synthetic polypeptide”
EVQVLESGGGLVQPGGSLRLSCAASGFTFSRFAMSWVRQAPGKGLEWVSAISGSAGSIDYADSVKGRFTISRDNSKNTLYLQMNSLRVEDTAVYYCADGTGPFFDYRGQGTLVTVSS

SEQ ID NO: 50 - protein, 117 aa, Artificial Sequence
  source: /note=“Description of Artificial Sequence: Synthetic polypeptide”
EVQVLESGGDVVQPGRSLRLSCAASGFTFSSYGMHWVRQAPGKEREWVAVISHDGSKKYYGDSVKGRFTISRDNSKNTLYLQMNSLRAEDTAVYYCADGTGPFFDYRGQGTLVTVSS

SEQ ID NO: 51 - protein, 117 aa, Artificial Sequence
  source: /note=“Description of Artificial Sequence: Synthetic polypeptide”
EVQVLESGGGVVQPGRSLRLSCAASGFTFSSYAMSWVRQAPGKEREWVSAISGSGGSTYYADSVKGRFTISRDNSKNTLYLQMNSLRAEDTAVYYCADGTGPFFDYRGQGTLVTVSS

SEQ ID NO: 52 - protein, 117 aa, Artificial Sequence
  source: /note=“Description of Artificial Sequence: Synthetic polypeptide”
EVQVLESGGGVVQPGRSLRLSCAASGFTFSSYGMHWVRQAPGKEREWVAVISYDGSNKYYADSVKGRFTISRDNSKNTLYLQMNSLRVEDTAVYYCADGTGPFFDYRGQGTLVTVSS

SEQ ID NO: 53 - protein, 117 aa, Artificial Sequence
  source: /note=“Description of Artificial Sequence: Synthetic polypeptide”
EVQVLESGGGLVQPGGSLRLSCAASGFTFSSYAMNWVRQAPGKGLEWVSSISGSGGTTDYADSVEGRFTISRDNSKNTLYLQMNSLRAEDTAVYYCADGTGPFFDYRGQGTLVTVSS

SEQ ID NO: 54 - protein, 117 aa, Artificial Sequence
  source: /note=“Description of Artificial Sequence: Synthetic polypeptide”
EVQVLESGGGLVEPGGSLRLSCAASGFMFSSYAMSWVRQAPGKGPNWVSAISGSGGSTSYADSVKGRFTISRDNSNNTLYLQMNSLRTEDTAVYYCADGTGPFFDYRGQGTLVTVSS

SEQ ID NO: 55 - protein, 117 aa, Artificial Sequence
  source: /note=“Description of Artificial Sequence: Synthetic polypeptide”
EVQVLESGGGLVQPGGSLRLSCAASGFIFSSYAMSWVRQAPGKGLEWVSGISGSGVNTYYADSVKGRFTISRDNSKNTLYLQMNSLRAEDTAVYYCADGTGPFFDYRGQGTLVTVSS

SEQ ID NO: 56 - protein, 117 aa, Artificial Sequence
  source: /note=“Description of Artificial Sequence: Synthetic polypeptide”
EVQVLESGGGLVQPGGSLRLSCAASRFTFSNFAMSWVRQAPGKGLEWVSAISDSGGSTYYADSVKGRFTISRDNSKNTLYLQMNSLRAEDTAVYYCADGTGPFFDYRGQGTLVTVSS

SEQ ID NO: 57 - protein, 117 aa, Artificial Sequence
  source: /note=“Description of Artificial Sequence: Synthetic polypeptide”
EVQVLESGGGLVQPGGSLRLSCAASGFTFSSYAMNWVRQAPGKGLEWVSAISGRGDSTYYADSVKGRFTISRDNSKNTLYLQMNSLRAEDTAVYNCADGRGPFFDYRGQGTLVTVSS

SEQ ID NO: 58 - protein, 117 aa, Artificial Sequence
  source: /note=“Description of Artificial Sequence: Synthetic polypeptide”
EVQVLESGGGLVQPGGSLRLSCAASGFMFSSYGMHWVRQAPGKEREWVAVISDDGSNKFYADSVKGRFTISRDNSKNTLYLQMNSLRAEDTAVYYCADGRGPFFDYRGQGTLVTVSS

SEQ ID NO: 59 - protein, 117 aa, Artificial Sequence
  source: /note=“Description of Artificial Sequence: Synthetic polypeptide”
EVQVLESGGGLVQPGGSLRLSCAASGFTFSSYVMSWVRQAPGKGLEWVSAISGSGVNTDYADSVKGRFTISRDNSKNTLYLQLNSLRAEDTAVYYCADGRGPFFDYRGQGTLVTVSS

SEQ ID NO: 60 - protein, 117 aa, Artificial Sequence
  source: /note=“Description of Artificial Sequence: Synthetic polypeptide”
EVQVLESGGGLVQPGGSLRLSCAASGFTFSSYGMHWVRQAPGKEREWVAVISYDGSNKYYADSVKGRFTISRDNSKNTLYLQMNSLRVEDTAVYYCADGKGPFFDYRGQGTLVTVSS

SEQ ID NO: 61 - protein, 8 aa, Artificial Sequence
  source: /note=“Description of Artificial Sequence: Synthetic peptide”
GFTFSNFA

SEQ ID NO: 62 - protein, 8 aa, Artificial Sequence
  source: /note=“Description of Artificial Sequence: Synthetic peptide”
GFTFTNFA

SEQ ID NO: 63 - protein, 8 aa, Artificial Sequence
  source: /note=“Description of Artificial Sequence: Synthetic peptide”
GFTFSSYA

SEQ ID NO: 64 - protein, 8 aa, Artificial Sequence
  source: /note=“Description of Artificial Sequence: Synthetic peptide”
GFTFSRYA

SEQ ID NO: 65 - protein, 8 aa, Artificial Sequence
  source: /note=“Description of Artificial Sequence: Synthetic peptide”
GFTFSNYA

SEQ ID NO: 66 - protein, 8 aa, Artificial Sequence
  source: /note=“Description of Artificial Sequence: Synthetic peptide”
GFTFSSFA

SEQ ID NO: 67 - protein, 8 aa, Artificial Sequence
  source: /note=“Description of Artificial Sequence: Synthetic peptide”
GFTFSSYG

SEQ ID NO: 68 - protein, 8 aa, Artificial Sequence
  source: /note=“Description of Artificial Sequence: Synthetic peptide”
GFIFSSYA

SEQ ID NO: 69 - protein, 8 aa, Artificial Sequence
  source: /note=“Description of Artificial Sequence: Synthetic peptide”
GFTFSGYG

SEQ ID NO: 70 - protein, 8 aa, Artificial Sequence
  source: /note=“Description of Artificial Sequence: Synthetic peptide”
GFTFSRFA

SEQ ID NO: 71 - protein, 8 aa, Artificial Sequence
  source: /note=“Description of Artificial Sequence: Synthetic peptide”
GFMFSSYA

SEQ ID NO: 72 - protein, 8 aa, Artificial Sequence
  source: /note=“Description of Artificial Sequence: Synthetic peptide”
RFTFSNFA

SEQ ID NO: 73 - protein, 8 aa, Artificial Sequence
  source: /note=“Description of Artificial Sequence: Synthetic peptide”
GFMFSSYG

SEQ ID NO: 74 - protein, 8 aa, Artificial Sequence
  source: /note=“Description of Artificial Sequence: Synthetic peptide”
GFTFSSYV

SEQ ID NO: 75 - protein, 8 aa, Artificial Sequence
  source: /note=“Description of Artificial Sequence: Synthetic peptide”
ISGSGGST

SEQ ID NO: 76 - protein, 8 aa, Artificial Sequence
  source: /note=“Description of Artificial Sequence: Synthetic peptide”
ISGGGGST

SEQ ID NO: 77 - protein, 8 aa, Artificial Sequence
  source: /note=“Description of Artificial Sequence: Synthetic peptide”
ISGSGDST

SEQ ID NO: 78 - protein, 8 aa, Artificial Sequence
  source: /note=“Description of Artificial Sequence: Synthetic peptide”
ISGSGGTT

SEQ ID NO: 79 - protein, 8 aa, Artificial Sequence
  source: /note=“Description of Artificial Sequence: Synthetic peptide”
ISGSAGTI

SEQ ID NO: 80 - protein, 8 aa, Artificial Sequence
  source: /note=“Description of Artificial Sequence: Synthetic peptide”
ISGSGGNT

SEQ ID NO: 81 - protein, 8 aa, Artificial Sequence
  source: /note=“Description of Artificial Sequence: Synthetic peptide”
ISNSGGST

SEQ ID NO: 82 - protein, 8 aa, Artificial Sequence
  source: /note=“Description of Artificial Sequence: Synthetic peptide”
ISGSAGRT

SEQ ID NO: 83 - protein, 8 aa, Artificial Sequence
  source: /note=“Description of Artificial Sequence: Synthetic peptide”
ISGGGGNT

SEQ ID NO: 84 - protein, 8 aa, Artificial Sequence
  source: /note=“Description of Artificial Sequence: Synthetic peptide”
ISGGGGGT

SEQ ID NO: 85 - protein, 8 aa, Artificial Sequence
  source: /note=“Description of Artificial Sequence: Synthetic peptide”
ISGSAGSI

SEQ ID NO: 86 - protein, 8 aa, Artificial Sequence
  source: /note=“Description of Artificial Sequence: Synthetic peptide”
ISYDGSNK

SEQ ID NO: 87 - protein, 8 aa, Artificial Sequence
  source: /note=“Description of Artificial Sequence: Synthetic peptide”
ISGSGSNT

SEQ ID NO: 88 - protein, 8 aa, Artificial Sequence
  source: /note=“Description of Artificial Sequence: Synthetic peptide”
ISNRGVNT

SEQ ID NO: 89 - protein, 8 aa, Artificial Sequence
  source: /note=“Description of Artificial Sequence: Synthetic peptide”
ISYDGGNK

SEQ ID NO: 90 - protein, 8 aa, Artificial Sequence
  source: /note=“Description of Artificial Sequence: Synthetic peptide”
ISYDGSKK

SEQ ID NO: 91 - protein, 8 aa, Artificial Sequence
  source: /note=“Description of Artificial Sequence: Synthetic peptide”
ISHDGSKK

SEQ ID NO: 92 - protein, 8 aa, Artificial Sequence
  source: /note=“Description of Artificial Sequence: Synthetic peptide”
ISGSGVNT

SEQ ID NO: 93 - protein, 8 aa, Artificial Sequence
  source: /note=“Description of Artificial Sequence: Synthetic peptide”
ISDSGGST

SEQ ID NO: 94 - protein, 8 aa, Artificial Sequence
  source: /note=“Description of Artificial Sequence: Synthetic peptide”
ISGRGDST

SEQ ID NO: 95 - protein, 8 aa, Artificial Sequence
  source: /note=“Description of Artificial Sequence: Synthetic peptide”
ISDDGSNK

SEQ ID NO: 96 - protein, 10 aa, Artificial Sequence
  source: /note=“Description of Artificial Sequence: Synthetic peptide”
ADGRGPFFDY

SEQ ID NO: 97 - protein, 10 aa, Artificial Sequence
  source: /note=“Description of Artificial Sequence: Synthetic peptide”
ADGTGPFFDY

SEQ ID NO: 98 - protein, 10 aa, Artificial Sequence
  source: /note=“Description of Artificial Sequence: Synthetic peptide”
ADGKGPFFDY

SEQ ID NO: 99 - protein, 122 aa, Artificial Sequence
  source: /note=“Description of Artificial Sequence: Synthetic polypeptide”
EVQLLESGGGLVQPGGSLRLSCAASGFIFSSYAMSWVRQAPGKGQEWVSGVSGTGVNTYYADSVKGRFTISRDNSKNTLYLQMNSLRAEDTAVYYCASGKGWDSGSWYEDYRGQGTLVTVSS

SEQ ID NO: 100 - protein, 122 aa, Artificial Sequence
  source: /note=“Description of Artificial Sequence: Synthetic polypeptide”
EVQLLESGGGLVQPGGSLRLSCAASGFTFSIYAMSWVRQAPGKGLEWVSGISGSGVNTYYADSVKGRFTISRDNSRNTVYLQMNSLRADDTAVYHCANGKGWDGNTWYEDYRGQGTLVTVSS

SEQ ID NO: 101 - protein, 122 aa, Artificial Sequence
  source: /note=“Description of Artificial Sequence: Synthetic polypeptide”
EVQLLESGGGLVQPGGSLRLSCAASGFIFSSYAMSWVRQAPGKGQEWVSGVSGSGVNTYYADSVKGRFTISRDNPKNTVYLQMNSLRAEDTAVYYCASGKGWDGNTWYEDYRGQGTLVTVSS

SEQ ID NO: 102 - protein, 122 aa, Artificial Sequence
  source: /note=“Description of Artificial Sequence: Synthetic polypeptide”
EVQLLESGGGLVQPGGSLRLSCAASGFIFSVYAMSWVRQAPGMGPEWVSGVSGSGINTYYADSVKGRFTISRDNSKNTVYLEMNSLRAEDTAVYYCASGKGWDGNTWYEDYRGQGTLVTVSS

SEQ ID NO: 103 - protein, 122 aa, Artificial Sequence
  source: /note=“Description of Artificial Sequence: Synthetic polypeptide”
EVQLLESGGGLVQPGGSLRLSCAASGFIFSIYAMSWVRQAPGKGLEWVSGISGSGANTYYADSVKGRFTISRDNSKNTLYLQMNSLRAEDTAVYYCASGKGWDGNTWYEDYRGQGTLVTVSS

SEQ ID NO: 104 - protein, 122 aa, Artificial Sequence
  source: /note=“Description of Artificial Sequence: Synthetic polypeptide”
EVQLLESGGGLVQPGGSLRLSCAASGFTFSIYAMSWVRQAPGKGLEWVSGISGSGVNTYYADSVKGRFTISRDNSKNTVYLQMNSLRAEDTAVYYCASGKGWDGNTWYEDYRGQGTLVTVSS

SEQ ID NO: 105 - protein, 122 aa, Artificial Sequence
  source: /note=“Description of Artificial Sequence: Synthetic polypeptide”
EVQLLESGGGLVQPGGSLRLSCAASGFIFSSYAMSWVRQAPGKGLEWVSAISGSGVRTYYADSVKGRFTISRDNSKNTLYLQMSSLRAEDTAVYSCASGKGWDGNTWYEDYRGQGTLVTVSS

SEQ ID NO: 106 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGVSGSGINTYYADSVKGRFTISRDNSKNTVYLEMNSLRAEDTAVYYCASGKGWDGNTWYEDYRGQGTLVTVSS

SEQ ID NO: 107 - protein, 122 aa, Artificial Sequence
  source: /note=“Description of Artificial Sequence: Synthetic polypeptide”
EVQLLESGGGLVQPGGSLRLSCAASGFTFSIYAMSWVRQAPGKGLEWVSGISGSGVNTYYADSVKGRFTISRDNSRNTVYLQMNSLRVEDTAVYYCASGKGWDGSTWYEDYRGQGTLVTVSS

SEQ ID NO: 108 - protein, 122 aa, Artificial Sequence
  source: /note=“Description of Artificial Sequence: Synthetic polypeptide”
EVQLLESGGGLVQPGGSLRLSCAASGFTFSIYAMSWVRQAPGKGLEWVSAISGSGVNTYYADSVKGRFTISRDNSKNTLYLHMNSLRAEDTAVYYCASGKGWDGNTWYEDYRGQGTLVTVSS

SEQ ID NO: 109 - protein, 122 aa, Artificial Sequence
  source: /note=“Description of Artificial Sequence: Synthetic polypeptide”
EVQLLESGGGLVQPGGSLRLSCTASGFIFSIYAMSWVRQAPGKGLEWVSAISGSGGSTYYADSVKGRFTISRDNSQNTLYLQMNSLRAEDTAVYYCASGKGWDGSTWYEDYRGQGTLVTVSS

SEQ ID NO: 110 - protein, 122 aa, Artificial Sequence
  source: /note=“Description of Artificial Sequence: Synthetic polypeptide”
EVQLLESGGGLVQPGGSLRLSCAASGFTFSIYAMSWVRQAPGKGLEWVSGISGTGINTYYADSVKGRFIISRDNSKNTLDLQMNSLRADDTAVYYCASGKGWDGNTWYEDYRGQGTLVTVSS

SEQ ID NO: 111 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGMGPEWVSGVSGSGVNTYYADSVKGRFTISRDNSKNTLYLQMNSLRAEDTAVYYCASGKGWDGNTWYEDYRGQGTLVTVSS

SEQ ID NO: 112 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGVSGSGVNTYYADSVKGRFTISRDNPKNTVYLQMNSLRADDTAVYFCASGKGWDGSTWYEDYRGQGTLVTVSS

SEQ ID NO: 113 - protein, 122 aa, Artificial Sequence
  source: /note=“Description of Artificial Sequence: Synthetic polypeptide”
EVQLLESGGGLVQPGGSLRLSCAASGFTFSIYAMSWVRQAPGKGLEWVSGVSGSGINTYYADSVKGRFTISRDNSKNTVYLEMNSLRAEDTAVYYCASGKGWDGNTWYEDYRGQGTLVTVSS

SEQ ID NO: 114 - protein, 122 aa, Artificial Sequence
  source: /note=“Description of Artificial Sequence: Synthetic polypeptide”
EVQLLESGGGMVQPGGSLRLSCAASGFTFSSYAMSWVRQAPGMGPEWVSGISGSGVNTYYADSVKGRFTISRDNSKNTLYLQMNSLRADDTAVYYCASGKGWDGNTWYEDYRGQGTLVTVSS

SEQ ID NO: 115 - protein, 122 aa, Artificial Sequence
  source: /note=“Description of Artificial Sequence: Synthetic polypeptide”
EVQLLESGGGLVQPGGSLRLSCAASGFIFSIYAMSWVRQAPGKGLEWVSGISGSGVNTYYADSVKGRFTISRDNSRNTVYLQMNSLRAEDTAVYYCARGKGWDGNTWYEDYRGQGTLVTVSS

SEQ ID NO: 116 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGISNRGVNTYYADNVKGRFIISRDNSKNTLDLQMNSLRADDTAVYYCASGKGWDGNTWYEDYRGQGTLVTVSS

SEQ ID NO: 117 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGISNTGINTYYADSVKGRFTISRDNSKNTLYLQMNSLRAEDTAVYYCASGKGWDGNTWYEDYRGQGTLVTVSS

SEQ ID NO: 118 - protein, 122 aa, Artificial Sequence
  source: /note=“Description of Artificial Sequence: Synthetic polypeptide”
EVQLLESGGGVVQPGRSLRLSCAASGFTFSSYAMSWVRQAPGKGLEWVSGISGSGVNTYYADSVKGRFIISRDNSKNTLYLQMNSLRAEDTAVYYCASGKGWDGNTWYEDYRGQGTLVTVSS

SEQ ID NO: 119 - protein, 122 aa, Artificial Sequence
  source: /note=“Description of Artificial Sequence: Synthetic polypeptide”
EVQLLESGGGLVQPGGSLRLSCAASGFIFSSYAMSWVRQAPGKGLEWVSGISNTGINTYYADSVKGRFIISRDNSKNTLDLQMNSLRADDTAVYYCASGKGWDGNTWYEDYRGQGTLVTVSS

SEQ ID NO: 120 - protein, 122 aa, Artificial Sequence
  source: /note=“Description of Artificial Sequence: Synthetic polypeptide”
EVQLLESGGGLVQPGGSLRLSCAASGFIFSSYAMSWVRQAPGQGLEWVSGISNTGVNTYYADSVKGRFTISRDNSKNMLYLQMNSLRAEDTAVYYCASGKGWDGNTWYEDYRGQGTLVTVSS

SEQ ID NO: 121 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGEGLEWVSAISGSGVNTYYADSVKGRFTISRDNSKNTLYLQMNSLRAEDTAVYYCASGKGWDGNTWYEDYRGQGTLVTVSS

SEQ ID NO: 122 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGISNTGINTYYADSVKGRFIISRDNSKNTLDLQMNSLRADDTAVYYCASGKGWDGNTWYEDYRGQGTLVTVSS

SEQ ID NO: 123 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWISGISNRGVNTYYADNVKGRFIISRDNSKNTLDLQMNSLRADDTAVYYCASGKGWDGNTWYEDYRGQGTLVTVSS

SEQ ID NO: 124 - protein, 122 aa, Artificial Sequence
  source: /note=“Description of Artificial Sequence: Synthetic polypeptide”
EVQLLESGGGLVQPGGSLRLSCAASGFIFSVYAMSWVRQAPGKGLEWVSGISGSGGSTHYADSVQGRFTISRDNSKNTLDLQMNSLRADDTAVYYCASGKGWDGNTWYEDYRGQGTLVTVSS

SEQ ID NO: 125 - protein, 122 aa, Artificial Sequence
  source: /note=“Description of Artificial Sequence: Synthetic polypeptide”
EVQLLESGGGLVQPGGSLRLSCTASGFIFSIYAMSWVRQAPGKGLEWVSGISNRGVNTYYADNVKGRFIISRDNSKNTLDLQMNSLRAEDTAVYYCASGKGWDGNTWYEDYRGQGTLVTVSS

SEQ ID NO: 126 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGISNTGINTYYADSVKGRFTISRDNSKNTLDLQMNSLRADDTAVYYCASGKGWDGNTWYEDYRGQGTLVTVSS

SEQ ID NO: 127 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYFCASGKGWDGNTWYEDYRGQGTLVTVSS

SEQ ID NO: 128 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTALYYCASGKGWDGNTWYEDYRGQGTLVTVSS

SEQ ID NO: 129 - protein, 122 aa, Artificial Sequence
  source: /note=“Description of Artificial Sequence: Synthetic polypeptide”
EVQLLESGGGLVQPGGSLRLSCAASGFTFSSYVMSWVRQAPGKGLEWVSGISGSGANTYYADSVKGRFTISRDNSKNTLYLQMNSLRAEDTAVYYCASGKGWDGNTWYEDYRGQGTLVTVSS

SEQ ID NO: 130 - protein, 119 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSNTWYEDYRGQGTLVTVSS

SEQ ID NO: 131 - protein, 122 aa, Artificial Sequence
  source: /note=“Description of Artificial Sequence: Synthetic polypeptide”
EVQLLESGGGVVQPGRSLRLSCAASGFTFSSYAMSWVRQAPGKGLEWVSAISGSGGSTYYADSVKGRFTISRDNSKNTVYLQMNSLRAEDTAVYYCASGKGWDGNTWYEDYRGQGTLVTVSS

SEQ ID NO: 132 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFIISRDNSKNTLDLQMNSLRADDTAVYYCASGKGWDGNTWYEDYRGQGTLVTVSS

SEQ ID NO: 133 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNPKNTVYLQMNSLRAEDTAVYYCASGKGWDGNTWYEDYRGQGTLVTVSS

SEQ ID NO: 134 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTVYLQMNSLRAEDTAVYYCASGKGWDGNTWYEDYRGQGTLVTVSS

SEQ ID NO: 135 - protein, 118 aa, Artificial Sequence
  source: /note=“Description of Artificial Sequence: Synthetic polypeptide”
EVQLLESGGGLVQPGGSLRLSCAASGFTFSVYAMSWVRQAPGMGPEWVSGISGSGVNTYYADSVKGRFTISRDNSKNTLYLQMNSLRAEDTAVYYCARHSNTWYEDYRGQGTLVTVSS

SEQ ID NO: 136 - skipped

SEQ ID NO: 137 - protein, 122 aa, Artificial Sequence
  source: /note=“Description of Artificial Sequence: Synthetic polypeptide”
EVQLLESGGGLVQPGGSLRLACAASGFTFSSYAMSWVRQAPGKGLEWVSSISGSGVNTYYADSVKGRFTISRDNSRNTLYLQMNSLRAEDTAVYYCASGKGWDGNTWYEDYRGQGTLVTVSS

SEQ ID NO: 138 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLERVSAISSSGGSTNYADSVKGRFTISRDNAKNTLDLQMNSLRADDTALYYCASGKGWDGNTWYEDYRGQGTLVTVSS

SEQ ID NO: 139 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLDLQMNSLRADDTAVYYCASGKGWDGNTWYEDYRGQGTLVTVSS

SEQ ID NO: 140 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRADDTALYYCASGKGWDGNTWYEDYRGQGTLVTVSS

SEQ ID NO: 141 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGVEWVSAISGSGGSTFYADSVKGRFTISRDNSKNTLYLQMFSLRAEDTAVYYCASGKGWDGNTWYEDYRGQGTLVTVSS

SEQ ID NO: 142 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASGKGWDGNTWYEDYRGQGTLVTVSS

SEQ ID NO: 143 - protein, 122 aa, Artificial Sequence
  source: /note=“Description of Artificial Sequence: Synthetic polypeptide”
EVQLLESGGGLVQPGGSLRLSCAASGFTFSSYGMHWVRQSPGKEREWVAVISYDGSNKYYSDSVKGRFTISRDNSKNTLYLQMNSLRADDTAVYYCASGKGWDGNTWYEDYRGQGTLVTVSS

SEQ ID NO: 144 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAITTSGGDTYYADSVNDRFTISRDNSKNTLYLQMYSLRAEDTAVYYCASGKGWDGNTWYEDYRGQGTLVTVSS

SEQ ID NO: 145 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSTISGSGANTYYADSVKGRFTISRDNSKNTLFLQMNSLRADDTAVYYCASGKGWDGNTWYEDYRGQGTLVTVSS

SEQ ID NO: 146 - protein, 122 aa, Artificial Sequence
  source: /note=“Description of Artificial Sequence: Synthetic polypeptide”
EVQLLESGGGLVQPGGSLRLSCAASGFTFSIYAMSWVRQAPGKGLEWISSISGNGGNTFYADSVKGRFTISRDNSKNTLYLQMNSLRAEDTAVYYCASGKGWDGNTWYEDYRGQGTLVTVSS

SEQ ID NO: 147 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSSISGTGDSTYYADSVKGRFTISRDNSNNTLDLQMNSLRADDTAVYYCASGKGWDGNTWYEDYRGQGTLVTVSS

SEQ ID NO: 148 - protein, 122 aa, Artificial Sequence
  source: /note=“Description of Artificial Sequence: Synthetic polypeptide”
EVQLLESGGGLVQPGGSLRVSCAASGFTFSSYAMSWVRQAPGKGLERLSAISGSGVNTYYADSVKGRFTISRNNSNNTLYLQMNSLRAEDTAVYYCASGKGWDGNTWYEDYRGQGTLVTVSS

SEQ ID NO: 149 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTNYVDSVKGRFIISRDNSKNTPDLQMNSLRADDTAVYYCASGKGWDGNTWYEDYRGQGTLVTVSS

SEQ ID NO: 150 - protein, 8 aa, Artificial Sequence
  source: /note=“Description of Artificial Sequence: Synthetic peptide”
GFTFSIYA

SEQ ID NO: 151 - protein, 8 aa, Artificial Sequence
  source: /note=“Description of Artificial Sequence: Synthetic peptide”
GFIFSSYA

SEQ ID NO: 152 - protein, 8 aa, Artificial Sequence
  source: /note=“Description of Artificial Sequence: Synthetic peptide”
GFIFSVYA

SEQ ID NO: 153 - protein, 8 aa, Artificial Sequence
  source: /note=“Description of Artificial Sequence: Synthetic peptide”
GFIFSIYA

SEQ ID NO: 154 - protein, 8 aa, Artificial Sequence
  source: /note=“Description of Artificial Sequence: Synthetic peptide”
GFTFSSYV

SEQ ID NO: 155 - protein, 8 aa, Artificial Sequence
  source: /note=“Description of Artificial Sequence: Synthetic peptide”
GFTFSVYA

SEQ ID NO: 156 - protein, 8 aa, Artificial Sequence
  source: /note=“Description of Artificial Sequence: Synthetic peptide”
VSGSGVNT

SEQ ID NO: 157 - protein, 8 aa, Artificial Sequence
  source: /note=“Description of Artificial Sequence: Synthetic peptide”
VSGSGINT

SEQ ID NO: 158 - protein, 8 aa, Artificial Sequence
  source: /note=“Description of Artificial Sequence: Synthetic peptide”
ISGSGANT

SEQ ID NO: 159 - protein, 8 aa, Artificial Sequence
  source: /note=“Description of Artificial Sequence: Synthetic peptide”
ISGSGVRT

SEQ ID NO: 160 - protein, 8 aa, Artificial Sequence
  source: /note=“Description of Artificial Sequence: Synthetic peptide”
ISGTGINT

SEQ ID NO: 161 - protein, 8 aa, Artificial Sequence
  source: /note=“Description of Artificial Sequence: Synthetic peptide”
ISNTGINT

SEQ ID NO: 162 - protein, 8 aa, Artificial Sequence
  source: /note=“Description of Artificial Sequence: Synthetic peptide”
ISNTGVNT

SEQ ID NO: 163 - protein, 8 aa, Artificial Sequence
  source: /note=“Description of Artificial Sequence: Synthetic peptide”
VSGTGVNT

SEQ ID NO: 164 - protein, 8 aa, Artificial Sequence
  source: /note=“Description of Artificial Sequence: Synthetic peptide”
ISSSGGST

SEQ ID NO: 165 - protein, 8 aa, Artificial Sequence
  source: /note=“Description of Artificial Sequence: Synthetic peptide”
ITTSGGDT

SEQ ID NO: 166 - protein, 8 aa, Artificial Sequence
  source: /note=“Description of Artificial Sequence: Synthetic peptide”
ISGNGGNT

SEQ ID NO: 167 - protein, 8 aa, Artificial Sequence
  source: /note=“Description of Artificial Sequence: Synthetic peptide”
ISGTGDST

SEQ ID NO: 168 - protein, 15 aa, Artificial Sequence
  source: /note=“Description of Artificial Sequence: Synthetic peptide”
ANGKGWDGNTWYEDY

SEQ ID NO: 169 - protein, 15 aa, Artificial Sequence
  source: /note=“Description of Artificial Sequence: Synthetic peptide”
ASGKGWDGNTWYEDY

SEQ ID NO: 170 - protein, 15 aa, Artificial Sequence
  source: /note=“Description of Artificial Sequence: Synthetic peptide”
ASGKGWDGSTWYEDY

SEQ ID NO: 171 - protein, 15 aa, Artificial Sequence
  source: /note=“Description of Artificial Sequence: Synthetic peptide”
ARGKGWDGNTWYEDY

SEQ ID NO: 172 - protein, 15 aa, Artificial Sequence
  source: /note=“Description of Artificial Sequence: Synthetic peptide”
ASGKGWDSGSWYEDY

SEQ ID NO: 173 - protein, 12 aa, Artificial Sequence
  source: /note=“Description of Artificial Sequence: Synthetic peptide”
AKGYSNTWYEDY

SEQ ID NO: 174 - protein, 11 aa, Artificial Sequence
  source: /note=“Description of Artificial Sequence: Synthetic peptide”
ARHSNTWYEDY

SEQ ID NO: 175 - protein, 118 aa, Artificial Sequence
  source: /note=“Description of Artificial Sequence: Synthetic polypeptide”
EVQLLESGGGLVQPGGSLRLSCAASGFTFSSYDMSWVRQAPGKGLEWVSTISGSSGNTYYADSVKGRFTISRDNSKNTLYLQMNSLRAEDTAVYYCANRRAAVAGDLWGQGTLVTVSS

SEQ ID NO: 176 - protein, 118 aa, Artificial Sequence
  source: /note=“Description of Artificial Sequence: Synthetic polypeptide”
EVQLLESGGGLVQPGGSLRLSCAASGFTLSNYDMTWVRQAPGKGLEWVSTISGRGGSTYYADSVKGRFTISRDNSKNTLYLQMNSLRVEDTAVYYCANRRIAVAGDLWGQGTLVTVSS

SEQ ID NO: 177 - protein, 118 aa, Artificial Sequence
  source: /note=“Description of Artificial Sequence: Synthetic polypeptide”
EVQLLESGGGLVQPGGSLRLSCAASGFTLSNYDMTWVRQAPGKGLEWVSTISGGGGNTYYADSVKGRFTISRDNSKNTLYLQMNSLRAEDTAVYYCANRRIAVAGDLWGQGTLVTVSS

SEQ ID NO: 178 - protein, 118 aa, Artificial Sequence
  source: /note=“Description of Artificial Sequence: Synthetic polypeptide”
EVQLLESGGGLVQPGGSLRLSCAASGFTLSNYDMTWVRQAPGKGLEWVSSISGSDGSTYYADSVKGRFTISRDNSKNTLYLQMNSLRAEDTAVYYCANRRAAVAGDLWGQGTLVTVSS

SEQ ID NO: 179 - protein, 118 aa, Artificial Sequence
  source: /note=“Description of Artificial Sequence: Synthetic polypeptide”
EVQLLESGGGVVQPGRSLRLSCAASGFTFSSYGMHWVRQAPGKEREWVAVISYDGSNKYYADSVKGRFTISRDNSKNTLYLQMNSLRAEDTAVYYCANRRAAVAGDLWGQGTLVTVSS

SEQ ID NO: 180 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSTISGSGDFTYYADSVKGRFTISRDISKNTLYLQMNSLRAEDTAVYYCANRRAAVAGDLWGQGTLVTVSS

SEQ ID NO: 181 - protein, 118 aa, Artificial Sequence
  source: /note=“Description of Artificial Sequence: Synthetic polypeptide”
EVQLLESGGGLVQPGGSLRLSCAASGFSLSSYTMSWVRQAPGKGLEWVSAISGSGGSTYYADSVKGRFTISRDNSKNTLFLQMNSLRVEDTAVYYCANRRAAVAGDLWGQGTLVTVSS

SEQ ID NO: 182 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLFLQMNSLRVEDTAVYYCANRRAAVAGDLWGQGTLVTVSS

SEQ ID NO: 183 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDISKNTLYLQMNSLRVEDTAVYYCANRRAAVAGDLWGQGTLVTVSS

SEQ ID NO: 184 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCANRRAAVAGDLWGQGTLVTVSS

SEQ ID NO: 185 - protein, 118 aa, Artificial Sequence
  source: /note=“Description of Artificial Sequence: Synthetic polypeptide”
EVQLLESGGGLVQPGGSLRLSCAASGFTFRSYGMSWVRQAPGKGLEWVSAISGSGGSTYYADSVKGRFTISRDNSKNTLFLQMNSLRVEDTAVYYCANRRAAVAGDLWGQGTLVTVSS

SEQ ID NO: 186 - protein, 117 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSTISGSSGNTYYADSVKGRFTISRDNSKNTLYLQMNSLRAEDTAVYYCANRRAAVAGVWGQGTLVTVSS

SEQ ID NO: 187 - protein, 118 aa, Artificial Sequence
  source: /note=“Description of Artificial Sequence: Synthetic polypeptide”
EVQLLESGGGLVQPGGSLRLSCAASGFTLSNYDMTWVRQAPGKGLEWVSTISGSGGSTAYADSVKGRFTISRDNSKNTLFLQMNSLRVEDTAVYYCANRRAAVAGDLWGQGTLVTVSS

SEQ ID NO: 188 - protein, 117 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEGAVAGDLWGQGTLVTVSS

SEQ ID NO: 189 - protein, 117 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGAVAGDLWGQGTLVTVSS

SEQ ID NO: 190 - protein, 118 aa, Artificial Sequence
  source: /note=“Description of Artificial Sequence: Synthetic polypeptide”
EVQLLESGGGVVQPGRSLRLSCAASGFTFSSYAMSWVRQAPGKGLEWVSTISGSGGNTYYADSVKGRFTISRDNSKNTLYLQMNSLRAEDTAVYYCANRRAAVAGDLWGQGTLVTVSS

SEQ ID NO: 191 - protein, 117 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSDGSTYYADSVKGRFTISRDNSKNTLYLQMNSLRAEDTAVYYCAKDQAVAGDLWGQGTLVTVSS

SEQ ID NO: 192 - protein, 118 aa, Artificial Sequence
  source: /note=“Description of Artificial Sequence: Synthetic polypeptide”
EVQLLESGGGLVQPGGSLRLSCAASGFTFSSYDMTWVRQAPGKGLEWVSTISGGGGNTYYADSVKGRFTISRDNSKNTLFLQMNSLRVEDTAVYYCANRRAAVAGDLWGQGTLVTVSS

SEQ ID NO: 193 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKEREWVAVISYDGSNKYYADSVKGRFTISRDNSKNTLYLQMNSLRADDTAVYYCANRRIAVAGDLWGQGTLVTVSS

SEQ ID NO: 194 - protein, 118 aa, Artificial Sequence
  source: /note=“Description of Artificial Sequence: Synthetic polypeptide”
EVQLLESGGGLVQPGGSLRLSCAASGFTFSTYGMHWVRQAPGKEREWVAVISYDGTNKYYANSVKGRFTISRDNSKNTLYLQMNSLRVEDTAVYYCANRRIAVAGDLWGQGTLVTVSS

SEQ ID NO: 195 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCANRRIAVAGDLWGQGTLVTVSS

SEQ ID NO: 196 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSTISGGGGNTYYADSVKGRFTISRDNSKNTLFLQMNSLRVEDTAVYYCANRRAAVAGDLWGQGTLVTVSS

SEQ ID NO: 197 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSTISGSGGNTYYADSVKGRFTISRDNSKNTLFLQMNSLRVEDTAVYYCANRRAAVAGDLWGQGTLVTVSS

SEQ ID NO: 198 - protein, 118 aa, Artificial Sequence
  source: /note=“Description of Artificial Sequence: Synthetic polypeptide”
EVQLLESGGGLVQPGGSLRLSCAASGFTFSSYDMSWVRQAPGKGLEWVSGITYSGGHTYYADSVKGRFTISRDNSKNTLFLQMNSLRVEDTAVYYCANRRAAVAGDLWGQGTLVTVSS

SEQ ID NO: 199 - protein, 118 aa, Artificial Sequence
  source: /note=“Description of Artificial Sequence: Synthetic polypeptide”
EVQLLESGGGLVQPGGSLRLSCAASGFTLSSYTMSWVRQAPGKEREWVAVISSDGSNKYYADSVKGRFTISRDNSKNTLYLQMNSLRAEDTAVYYCANRRAAVAGDLWGQGTLVTVSS

SEQ ID NO: 200 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGDSTHYADSVKGRFTISRDNSKNTLFLQMNSLRVEDTAVYYCANRRAAVAGDLWGQGTLVTVSS

SEQ ID NO: 201 - protein, 118 aa, Artificial Sequence
  source: /note=“Description of Artificial Sequence: Synthetic polypeptide”
EVQLLESGGGVVQPGRSLRLSCAASGFTFSSKGMHWVRQAPGKEREWVAVISYDGSNKYYADSVKGRFTISRDNSKNTLYLQMNSLRAEDTAVYYCANRRIAVAGDLWGQGTLVTVSS

SEQ ID NO: 202 - protein, 118 aa, Artificial Sequence
  source: /note=“Description of Artificial Sequence: Synthetic polypeptide”
EVQLLESGGGLVQPGGSLRLSCAASGFTFSSYDMSWVRQAPGKGLEWVSTISGGGGNTYYADSVKGRFTISRDNSKNTLFLQMNSLRVEDTAVYYCANRRAAVAGDLWGQGTLVTVSS

SEQ ID NO: 203 - protein, 117 aa, Artificial Sequence
  source: /note=“Description of Artificial Sequence: Synthetic polypeptide”
EVQLLESGGGVVQPGRSLRLSCAASGFTFSSYGMHWVRQAPGKEREWVSSISGSDGSTYYADSVKGRFTISRDNSKNTLYLQMNSLRAEDTAVYYCAKDTAVAGDLWGQGTLVTVSS

SEQ ID NO: 204 - protein, 118 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSAISGSGGSTFYADSVKGRFTISRDNSKNTLYLQMNSLRAEDTAVYYCANRRIAVAGDLWGQGTLVTVSS

SEQ ID NO: 205 - protein, 117 aa, Artificial Sequence
  source: /note=“Description of Artificial Sequence: Synthetic polypeptide”
EVQLLESGGGLVQPGGSLRLSCAASGFTFSSYDMTWVRQAPGKGLEWVSTISGGGGNTYYADSVKGRFTISRDNSKNTLFLQMNSLRVEDTAVYYCANRRAAVAGDWGQGTLVTVSS

SEQ ID NO: 206 - protein, 118 aa, Artificial Sequence
  source: /note=“Description of Artificial Sequence: Synthetic polypeptide”
EVQLLESGGGLVQPGGSLRLSCAAAGFTFSSKGMHWVRQAPGKEREWVAVISYDGSNKYYADSVKGRFTISRDNSNNTLYLQMNSLRAEDTAVYYCANRRAAVAGDLWGQGTLVTVSS

SEQ ID NO: 207 - protein, 117 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGISGSGGSTYYADSVKGRFTISRDNSKNTLYLQMNSLRDEDTAVYYCAKDIAVAGDLWGQGTLVTVSS

SEQ ID NO: 208 - protein, 118 aa, Artificial Sequence; identical to SEQ ID NO: 175
  source: /note=“Description of Artificial Sequence: Synthetic polypeptide”

SEQ ID NO: 209 - protein, 118 aa, Artificial Sequence; identical to SEQ ID NO: 176
  source: /note=“Description of Artificial Sequence: Synthetic polypeptide”

SEQ ID NO: 210 - protein, 118 aa, Artificial Sequence; identical to SEQ ID NO: 177
  source: /note=“Description of Artificial Sequence: Synthetic polypeptide”

SEQ ID NO: 211 - protein, 118 aa, Artificial Sequence; identical to SEQ ID NO: 178
  source: /note=“Description of Artificial Sequence: Synthetic polypeptide”

SEQ ID NO: 212 - protein, 118 aa, Artificial Sequence; identical to SEQ ID NO: 179
  source: /note=“Description of Artificial Sequence: Synthetic polypeptide”

SEQ ID NO: 213 - protein, 118 aa, Artificial Sequence; identical to SEQ ID NO: 180
  source: /note=“Description of Artificial Sequence: Synthetic polypeptide”

SEQ ID NO: 214 - protein, 118 aa, Artificial Sequence; identical to SEQ ID NO: 181
  source: /note=“Description of Artificial Sequence: Synthetic polypeptide”

SEQ ID NO: 215 - protein, 118 aa, Artificial Sequence; identical to SEQ ID NO: 182
  source: /note=“Description of Artificial Sequence: Synthetic polypeptide”

SEQ ID NO: 216 - protein, 118 aa, Artificial Sequence; identical to SEQ ID NO: 183
  source: /note=“Description of Artificial Sequence: Synthetic polypeptide”

SEQ ID NO: 217 - protein, 118 aa, Artificial Sequence; identical to SEQ ID NO: 184
  source: /note=“Description of Artificial Sequence: Synthetic polypeptide”

SEQ ID NO: 218 - protein, 118 aa, Artificial Sequence; identical to SEQ ID NO: 185
  source: /note=“Description of Artificial Sequence: Synthetic polypeptide”

SEQ ID NO: 219 - protein, 8 aa, Artificial Sequence
  source: /note=“Description of Artificial Sequence: Synthetic peptide”
GFTLSNYD

SEQ ID NO: 220 - protein, 8 aa, Artificial Sequence
  source: /note=“Description of Artificial Sequence: Synthetic peptide”
GFTLSSYD

SEQ ID NO: 221 - protein, 8 aa, Artificial Sequence
  source: /note=“Description of Artificial Sequence: Synthetic peptide”
GFTLSSYT

SEQ ID NO: 222 - protein, 8 aa, Artificial Sequence
  source: /note=“Description of Artificial Sequence: Synthetic peptide”
GFTFSSYD

SEQ ID NO: 223 - protein, 8 aa, Artificial Sequence
  source: /note=“Description of Artificial Sequence: Synthetic peptide”
GFSLSSYT

SEQ ID NO: 224 - protein, 8 aa, Artificial Sequence
  source: /note=“Description of Artificial Sequence: Synthetic peptide”
GFTLRNYD

SEQ ID NO: 225 - protein, 8 aa, Artificial Sequence
  source: /note=“Description of Artificial Sequence: Synthetic peptide”
GFIVSSYD

SEQ ID NO: 226 - protein, 8 aa, Artificial Sequence
  source: /note=“Description of Artificial Sequence: Synthetic peptide”
GFTVSSYD

SEQ ID NO: 227 - protein, 8 aa, Artificial Sequence
  source: /note=“Description of Artificial Sequence: Synthetic peptide”
GFTFSSYA

SEQ ID NO: 228 - protein, 8 aa, Artificial Sequence
  source: /note=“Description of Artificial Sequence: Synthetic peptide”
GFTFRSYG

SEQ ID NO: 229 - protein, 8 aa, Artificial Sequence
  source: /note=“Description of Artificial Sequence: Synthetic peptide”
GFTFSTYG

SEQ ID NO: 230 - protein, 8 aa, Artificial Sequence
  source: /note=“Description of Artificial Sequence: Synthetic peptide”
GFTFSSKG

SEQ ID NO: 231 - protein, 8 aa, Artificial Sequence
  source: /note=“Description of Artificial Sequence: Synthetic peptide”
ISGRDGST

SEQ ID NO: 232 - protein, 8 aa, Artificial Sequence
  source: /note=“Description of Artificial Sequence: Synthetic peptide”
ISGSDGST

SEQ ID NO: 233 - protein, 8 aa, Artificial Sequence
  source: /note=“Description of Artificial Sequence: Synthetic peptide”
ISGSSGNT

SEQ ID NO: 234 - protein, 8 aa, Artificial Sequence
  source: /note=“Description of Artificial Sequence: Synthetic peptide”
ISGRGGST

SEQ ID NO: 235 - protein, 8 aa, Artificial Sequence
  source: /note=“Description of Artificial Sequence: Synthetic peptide”
ISGSGDFT

SEQ ID NO: 236 - protein, 8 aa, Artificial Sequence
  source: /note=“Description of Artificial Sequence: Synthetic peptide”
ISYDGTNK

SEQ ID NO: 237 - protein, 8 aa, Artificial Sequence
  source: /note=“Description of Artificial Sequence: Synthetic peptide”
ITYSGGHT

SEQ ID NO: 238 - protein, 8 aa, Artificial Sequence
  source: /note=“Description of Artificial Sequence: Synthetic peptide”
ISSDGSNK

SEQ ID NO: 239 - protein, 8 aa, Artificial Sequence
  source: /note=“Description of Artificial Sequence: Synthetic peptide”
ISGSGDST

SEQ ID NO: 240 - protein, 11 aa, Artificial Sequence
  source: /note=“Description of Artificial Sequence: Synthetic peptide”
ANRRAAVAGDL

SEQ ID NO: 241 - protein, 11 aa, Artificial Sequence
  source: /note=“Description of Artificial Sequence: Synthetic peptide”
ANRRIAVAGDL

SEQ ID NO: 242 - protein, 10 aa, Artificial Sequence
  source: /note=“Description of Artificial Sequence: Synthetic peptide”
ANRRAAVAGV

SEQ ID NO: 243 - protein, 10 aa, Artificial Sequence
  source: /note=“Description of Artificial Sequence: Synthetic peptide”
AKEGAVAGDL

SEQ ID NO: 244 - protein, 10 aa, Artificial Sequence
  source: /note=“Description of Artificial Sequence: Synthetic peptide”
AKDGAVAGDL

SEQ ID NO: 245 - protein, 10 aa, Artificial Sequence
  source: /note=“Description of Artificial Sequence: Synthetic peptide”
AKDQAVAGDL

SEQ ID NO: 246 - protein, 10 aa, Artificial Sequence
  source: /note=“Description of Artificial Sequence: Synthetic peptide”
AKDTAVAGDL

SEQ ID NO: 247 - protein, 10 aa, Artificial Sequence
  source: /note=“Description of Artificial Sequence: Synthetic peptide”
ANRRAAVAGD

SEQ ID NO: 248 - protein, 10 aa, Artificial Sequence
  source: /note=“Description of Artificial Sequence: Synthetic peptide”
AKDIAVAGDL

SEQ ID NO: 249 - protein, 119 aa, Artificial Sequence
  source: /note=“Description of Artificial Sequence: Synthetic polypeptide”
EVQVLESGGGLVQPGGSLRLSCAASGFTFSSSDMSWVRQAPGKGLEWVSAISGSGGNTYYADSVKGRFTISRDNSKNTLYLQMNSLRAEDTAVYYCALGGSSGPFFDNRGQGTLVTVSS

SEQ ID NO: 250 - protein, 119 aa, Artificial Sequence
  source: /note=“Description of Artificial Sequence: Synthetic polypeptide”
EVQVLESGGGLVQPGGSLRLSCAASGFTFSSNAMSWVRQAPGKGLEWVSAISSSGVSTSYADSVKGRFTISRDNSKNSLYLQMNSLRAEDTAVYYCALGGGSGPFFDNRGQGTLVTVSS

SEQ ID NO: 251 - protein, 119 aa, Artificial Sequence
  source: /note=“Description of Artificial Sequence: Synthetic polypeptide”
EVQVLESGGGLVQPGGSLRLSCAASGFTFRSYAMSWVRQAPGKGLEWVSAISGSGGSTYYADSVKGRFTISRDNSKSMLYLQMNSLRAEDTAVYYCSLGSSSGPFFDNRGQGTLVTVSS

SEQ ID NO: 252 - protein, 121 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRDIDSSGPFFDNRGQGTLVTVSS

SEQ ID NO: 253 - protein, 119 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGDNTDYADSVKGRFTISRDNSKNTLYLQMNSLRADDTAVYYCALGGSSGPFFDNRGQGTLVTVSS

SEQ ID NO: 254 - protein, 119 aa, Artificial Sequence
  source: /note=“Description of Artificial Sequence: Synthetic polypeptide”
EVQVLESGGGLVQPGGSLRLSCAASGFTFSSFAMSWVRQAPGKGLEWVSAISDSGGRTYYADSVKGRFTISRDNSNNTLYLQMDSLRAEDTAVYYCASGGSSGPFFDNRGQGTLVTVSS

SEQ ID NO: 255 - protein, 119 aa, Artificial Sequence
  source: /note=“Description of Artificial Sequence: Synthetic polypeptide”
EVQVLESGGGLVQPGGSLRLSCAASGFTFSSFAMSWVRQAPGKGLEWVAAISNSGASTDYEDSVKGRFTISRDNSKNTLYLHMNSLRVEDTALYYCALGSSSGPFFDNRGQGTLVTVSS

SEQ ID NO: 256 - protein, 119 aa, Artificial Sequence
  source: /note=“Description of Artificial Sequence: Synthetic polypeptide”
EVQVLESGGGLVQPGGSLRLSCAASGFTFSSHAMSWVRQAPGKGLEWVSTISGSGDSTSYADSVKGRFTISRDNSKNTLYLQMNSLRAEDTAVYYCALGGGSGPFFDNRGQGTLVTVSS

SEQ ID NO: 257 - protein, 119 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGTGDNTYYADSVKGRFTISRDNSKNTLSLQMNSLRAEDTAVYYCALGGSSGPFFDNRGQGTLVTVSS

SEQ ID NO: 258 - protein, 119 aa, Artificial Sequence
  source: /note=“Description of Artificial Sequence: Synthetic polypeptide”
EVQVLESGGGLVQPGGSLRLSCVASGFTFSSYGMSWVRQAPGKEREWVAAISGSGGNTYYADSVKGRFTISRDNSKNTLYLQMNSLRAEDTAVYYCALGGGSGPFFDNRGQGTLVTVSS

SEQ ID NO: 259 - protein, 119 aa, Artificial Sequence
  source: /note=“Description of Artificial Sequence: Synthetic polypeptide”
EVQVLESGGGLVQPGGSLRLSCAASGFTFSSYGMHWVRRAPGKEREWVAVISYDGSNKNYADSVKGRFTISRDNSKNTLYLQMNSLRAEDTAVYYCALGGSSGPFFDNRGQGTLVTVSS

SEQ ID NO: 260 - protein, 119 aa, Artificial Sequence
  source: /note=“Description of Artificial Sequence: Synthetic polypeptide”
EVQVLESGGGLVQPGGSLRLSCAASGFTFSSYVMSWVRQAPGKGLEWVSSISNSGGSTNYADSVKGRFTLSRDNSKNTLYLQMNSLRAEDTAVYYCALGGGSGPFFDNRGQGTLVTVSS

SEQ ID NO: 261 - protein, 119 aa, Artificial Sequence
  source: /note=“Description of Artificial Sequence: Synthetic polypeptide”
EVQVLESGGGLVQPGGSLRLSCAASGFTFSSYAMMWLRQAPGKGLEWVSGISDRGGSTHYEDSVKGRFTISRDNSKNTLYLQMNSLRAEDTAVYYCALGGSSGPFFDNRGQGTLVTVSS

SEQ ID NO: 262 - protein, 119 aa, Artificial Sequence
  source: /note=“Description of Artificial Sequence: Synthetic polypeptide”
EVQVLESGGGLVQPGGSLRLSCAVSGFTFSSYAMSWVRQAPGKGLEWVSAISSSGGSTNYADSVRGRFTISRDNSKNTLFLQMNSLRAEDTAMYYCALGGSSGPFFDNRGQGTLVTVSS

SEQ ID NO: 263 - protein, 119 aa, Artificial Sequence
  source: /note=“Description of Artificial Sequence: Synthetic polypeptide”
EVQVLESGGGLVQPGGSLRLSCAASGFTFHNWPMTWVRQAPGKRLEWVSAISGSGDSTYYADSVKGRFTISRDNSRNTLYLQMNSLRAEDTAVYYCALGGGSGPFFDNRGQGTLVTVSS

SEQ ID NO: 264 - protein, 120 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDQGSSGPFFDNRGQGTLVTVSS

SEQ ID NO: 265 - protein, 119 aa, Artificial Sequence
  source: /note=“Description of Artificial Sequence: Synthetic polypeptide”
EVQVLESGGGLVQPGGSLRLSCAASGFTFSSYGMHWVRQAPGKGLEWVSSISGSGGSTNYADSVKGRFTISRDNSKNTLFLQLNSLRAEDTAVYYCALGGGSGPFFDNRGQGTLVTVSS

SEQ ID NO: 266 - protein, 119 aa, Artificial Sequence
  source: /note=“Description of Artificial Sequence: Synthetic polypeptide”
EVQVLESGGGLIHPGGSLRLSCAASGFTFSTYAMTWIRQAPGKGLEWVSAISGSGDSPNYADSVKGRFTISRDNSKNTLYLQMNSLRAEDTAVYYCALGGSSGPFFDNRGQGTLVTVSS

SEQ ID NO: 267 - protein, 119 aa, Artificial Sequence
  source: /note=“Description of Artificial Sequence: Synthetic polypeptide”
EVQVLESGGGLVQPGGSLRLSCAASGFTYSSYAMTWIRQAPGKGLEWVSAISGSGGSTNYADFVKGRFTISRDNSKNTLYLQMNSLRAEDTAVYYCALGAGSGPFFDNRGQGTLVTVSS

SEQ ID NO: 268 - protein, 119 aa, Artificial Sequence
  source: /note=“Description of Artificial Sequence: Synthetic polypeptide”
EVQVLESGGGLVQPGGSLRLSCEASGFTFSSYTMKWVRQAPGKGLEWVSAISDSGGSTYYADSVKGRFTISRDNSKNTLYLQMNSLRAEDTAVYYCTKEDSSGPFFDNRGQGTLVTVSS

SEQ ID NO: 269 - protein, 119 aa, Artificial Sequence
  source: /note=“Description of Artificial Sequence: Synthetic polypeptide”
EVQVLESGGGLVQPGGSLRLSCAASGFTFSSYGMHWVRQAPGKEREWVAVISYDGNTIYYADSVKGRFTISRDNSKNTLYLQLNSLKSEDTAVYYCALGAGSGPFFDNRGQGTLVTVSS

SEQ ID NO: 270 - protein, 119 aa, Artificial Sequence
  source: /note=“Description of Artificial Sequence: Synthetic polypeptide”
EVQVLESGGGLVQPGGSLRLSCAASGFTFSSYAMKWVRQAPGKGLEWVSGISNSGGSTYYADSVKGRFTISRDNSRNTLYLQMNSLRAEDTAVYYCALGGGSGPFFDNRGQGTLVTVSS

SEQ ID NO: 271 - protein, 119 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISSSGASTNYADSVKGRFTISRDNSKNTLYLQMTSLRAEDTAVYYCSLGGSSGPFFDNRGQGTLVTVSS

SEQ ID NO: 272 - protein, 119 aa, Artificial Sequence
  source: /note=“Description of Artificial Sequence: Synthetic polypeptide”
EVQVLESGGGLVQPGGSLRLSCAASGFTFSSYAMSWVRQAPGKGLEWVSAISGSGGSTYYADTVKGRFTISRDNSKNALYLQMNSLRAEDTAVYYCALGGSSGPFFDNRGQGTLVTVSS

SEQ ID NO: 273 - protein, 119 aa, Artificial Sequence
  source: /note=“Description of Artificial Sequence: Synthetic polypeptide”
EVQVLESGGGLVQPGGSLRLSCTASGFTFSSYAMSWVRQAPGKGLKWVSAISSSGGSTSYADSVKGRFTISRDNSKNTLYLQMNSLRVEDTAVYYCALGSGSGPFFDNRGQGTLVTVSS

SEQ ID NO: 274 - protein, 119 aa, Artificial Sequence; identical to SEQ ID NO: 249
  source: /note=“Description of Artificial Sequence: Synthetic polypeptide”

SEQ ID NO: 275 - protein, 119 aa, Artificial Sequence; identical to SEQ ID NO: 250
  source: /note=“Description of Artificial Sequence: Synthetic polypeptide”

SEQ ID NO: 276 - protein, 119 aa, Artificial Sequence; identical to SEQ ID NO: 251
  source: /note=“Description of Artificial Sequence: Synthetic polypeptide”

SEQ ID NO: 277 - protein, 121 aa, Artificial Sequence; identical to SEQ ID NO: 252
  source: /note=“Description of Artificial Sequence: Synthetic polypeptide”

SEQ ID NO: 278 - protein, 119 aa, Artificial Sequence; identical to SEQ ID NO: 253
  source: /note=“Description of Artificial Sequence: Synthetic polypeptide”

SEQ ID NO: 279 - protein, 119 aa, Artificial Sequence; identical to SEQ ID NO: 254
  source: /note=“Description of Artificial Sequence: Synthetic polypeptide”

SEQ ID NO: 280 - protein, 119 aa, Artificial Sequence; identical to SEQ ID NO: 255
  source: /note=“Description of Artificial Sequence: Synthetic polypeptide”

SEQ ID NO: 281 - protein, 119 aa, Artificial Sequence; identical to SEQ ID NO: 256
  source: /note=“Description of Artificial Sequence: Synthetic polypeptide”

SEQ ID NO: 282 - protein, 119 aa, Artificial Sequence; identical to SEQ ID NO: 257
  source: /note=“Description of Artificial Sequence: Synthetic polypeptide”

SEQ ID NO: 283 - protein, 119 aa, Artificial Sequence; identical to SEQ ID NO: 258
  source: /note=“Description of Artificial Sequence: Synthetic polypeptide”

SEQ ID NO: 284 - protein, 119 aa, Artificial Sequence; identical to SEQ ID NO: 259
  source: /note=“Description of Artificial Sequence: Synthetic polypeptide”

SEQ ID NO: 285 - protein, 119 aa, Artificial Sequence; identical to SEQ ID NO: 260
  source: /note=“Description of Artificial Sequence: Synthetic polypeptide”

SEQ ID NO: 286 - protein, 119 aa, Artificial Sequence; identical to SEQ ID NO: 261
  source: /note=“Description of Artificial Sequence: Synthetic polypeptide”

SEQ ID NO: 287 - protein, 119 aa, Artificial Sequence; identical to SEQ ID NO: 262
  source: /note=“Description of Artificial Sequence: Synthetic polypeptide”

SEQ ID NO: 288 - protein, 119 aa, Artificial Sequence; identical to SEQ ID NO: 263
  source: /note=“Description of Artificial Sequence: Synthetic polypeptide”

SEQ ID NO: 289 - protein, 120 aa, Artificial Sequence; identical to SEQ ID NO: 264
  source: /note=“Description of Artificial Sequence: Synthetic polypeptide”

SEQ ID NO: 290 - protein, 119 aa, Artificial Sequence; identical to SEQ ID NO: 265
  source: /note=“Description of Artificial Sequence: Synthetic polypeptide”

SEQ ID NO: 291 - protein, 119 aa, Artificial Sequence; identical to SEQ ID NO: 266
  source: /note=“Description of Artificial Sequence: Synthetic polypeptide”

SEQ ID NO: 292 - protein, 119 aa, Artificial Sequence; identical to SEQ ID NO: 267
  source: /note=“Description of Artificial Sequence: Synthetic polypeptide”

SEQ ID NO: 293 - protein, 119 aa, Artificial Sequence; identical to SEQ ID NO: 268
  source: /note=“Description of Artificial Sequence: Synthetic polypeptide”

SEQ ID NO: 294 - protein, 119 aa, Artificial Sequence; identical to SEQ ID NO: 269
  source: /note=“Description of Artificial Sequence: Synthetic polypeptide”

SEQ ID NO: 295 - protein, 119 aa, Artificial Sequence; identical to SEQ ID NO: 270
  source: /note=“Description of Artificial Sequence: Synthetic polypeptide”

SEQ ID NO: 296 - protein, 119 aa, Artificial Sequence; identical to SEQ ID NO: 271
  source: /note=“Description of Artificial Sequence: Synthetic polypeptide”

SEQ ID NO: 297 - protein, 119 aa, Artificial Sequence; identical to SEQ ID NO: 272
  source: /note=“Description of Artificial Sequence: Synthetic polypeptide”

SEQ ID NO: 298 - protein, 119 aa, Artificial Sequence; identical to SEQ ID NO: 273
  source: /note=“Description of Artificial Sequence: Synthetic polypeptide”

SEQ ID NO: 299 - protein, 119 aa, Artificial Sequence; identical to SEQ ID NO: 249
  source: /note=“Description of Artificial Sequence: Synthetic polypeptide”

SEQ ID NO: 300 - protein, 119 aa, Artificial Sequence; identical to SEQ ID NO: 250
  source: /note=“Description of Artificial Sequence: Synthetic polypeptide”

SEQ ID NO: 301 - protein, 119 aa, Artificial Sequence; identical to SEQ ID NO: 251
  source: /note=“Description of Artificial Sequence: Synthetic polypeptide”

SEQ ID NO: 302 - protein, 121 aa, Artificial Sequence; identical to SEQ ID NO: 252
  source: /note=“Description of Artificial Sequence: Synthetic polypeptide”

SEQ ID NO: 303 - protein, 119 aa, Artificial Sequence; identical to SEQ ID NO: 253
  source: /note=“Description of Artificial Sequence: Synthetic polypeptide”

SEQ ID NO: 304 - protein, 119 aa, Artificial Sequence; identical to SEQ ID NO: 254
  source: /note=“Description of Artificial Sequence: Synthetic polypeptide”

SEQ ID NO: 305 - protein, 119 aa, Artificial Sequence; identical to SEQ ID NO: 255
  source: /note=“Description of Artificial Sequence: Synthetic polypeptide”

SEQ ID NO: 306 - protein, 119 aa, Artificial Sequence; identical to SEQ ID NO: 256
  source: /note=“Description of Artificial Sequence: Synthetic polypeptide”

SEQ ID NO: 307 - protein, 119 aa, Artificial Sequence; identical to SEQ ID NO: 257
  source: /note=“Description of Artificial Sequence: Synthetic polypeptide”

SEQ ID NO: 308 - protein, 119 aa, Artificial Sequence; identical to SEQ ID NO: 258
  source: /note=“Description of Artificial Sequence: Synthetic polypeptide”

SEQ ID NO: 309 - protein, 8 aa, Artificial Sequence
  source: /note=“Description of Artificial Sequence: Synthetic peptide”
GFTFSRFP

SEQ ID NO: 310 - protein, 8 aa, Artificial Sequence
  source: /note=“Description of Artificial Sequence: Synthetic peptide”
GFTFGNYA

SEQ ID NO: 311 - protein, 8 aa, Artificial Sequence
  source: /note=“Description of Artificial Sequence: Synthetic peptide”
GFPFSSYA

SEQ ID NO: 312 - protein, 8 aa, Artificial Sequence
  source: /note=“Description of Artificial Sequence: Synthetic peptide”
GFTFRSYA

SEQ ID NO: 313 - protein, 8 aa, Artificial Sequence
  source: /note=“Description of Artificial Sequence: Synthetic peptide”
GFTFSSSD

SEQ ID NO: 314 - protein, 8 aa, Artificial Sequence
  source: /note=“Description of Artificial Sequence: Synthetic peptide”
GFTFSSNA

SEQ ID NO: 315 - protein, 8 aa, Artificial Sequence
  source: /note=“Description of Artificial Sequence: Synthetic peptide”
GFTFSSHA

SEQ ID NO: 316 - protein, 8 aa, Artificial Sequence
  source: /note=“Description of Artificial Sequence: Synthetic peptide”
GFTFHNWP

SEQ ID NO: 317 - protein, 8 aa, Artificial Sequence
  source: /note=“Description of Artificial Sequence: Synthetic peptide”
GFTFSTYA

SEQ ID NO: 318 - protein, 8 aa, Artificial Sequence
  source: /note=“Description of Artificial Sequence: Synthetic peptide”
GFTYSSYA

SEQ ID NO: 319 - protein, 8 aa, Artificial Sequence
  source: /note=“Description of Artificial Sequence: Synthetic peptide”
GFTFSSYT

SEQ ID NO: 320 - protein, 8 aa, Artificial Sequence
  source: /note=“Description of Artificial Sequence: Synthetic peptide”
ISSSGAST

SEQ ID NO: 321 - protein, 8 aa, Artificial Sequence
  source: /note=“Description of Artificial Sequence: Synthetic peptide”
ISGSGDSP

SEQ ID NO: 322 - protein, 8 aa, Artificial Sequence
  source: /note=“Description of Artificial Sequence: Synthetic peptide”
ISGSGAST

SEQ ID NO: 323 - protein, 124 aa, Artificial Sequence
  source: /note=“Description of Artificial Sequence: Synthetic polypeptide”
QLQLQESGPGLVKPSETLALTCNVSGGSISSSSYYWGWIRQPPGKRLEWIGSIYYSGSTYFNPSLKSRVTISVDTSKNQFSLKLSSVTAADTAVYYCAGGDYYGPVGQWPSDYRGQGTLVTVSS

SEQ ID NO: 324 - protein, 124 aa, Artificial Sequence
  source: /note=“Description of Artificial Sequence: Synthetic polypeptide”
QLQLQESGPGLVKPSETLSLTCTVSGDSISSSDYYWGWIRQPPGKRLEWIGSIYHSGSTDYNPSLKSRVTIFVDTSKNQFSLKLTSVTAADTAVYYCAGGYRYGPVGQWPSDYRGQGTLVTVSS

SEQ ID NO: 325 - protein, 124 aa, Artificial Sequence
  source: /note=“Description of Artificial Sequence: Synthetic polypeptide”
QLQLQESGPGLVKPSETLSLTCTVSGGSVSSSSYYWGWIRQPPGKRLELIGSIYYGGSTYFNPSLKSRVTISVDTSKNQFSLKLSSVTAADTAVYYCAGGDTYGPVGQWPSDYRGQGTLVTVSS

SEQ ID NO: 326 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTNYNPSLKSRVTISVDTSKNQFSLKLSSVTAADTAVYYCAGGYNYGPVGQWPSDYRGQGTLVTVSS

SEQ ID NO: 327 - protein, 124 aa, Artificial Sequence
  source: /note=“Description of Artificial Sequence: Synthetic polypeptide”
QLQLQESGPGLVKPSETLSLTCTVSGASISSSSYYWGWIRQPPGKRLEWIGSIYYSGSTDYNPSLKSRVTISVDTSKNQFSLKLTSVTAADTAVYYCAGGYNYGPVGQWPSDYRGQGTLVTVSS

SEQ ID NO: 328 - protein, 124 aa, Artificial Sequence
  source: /note=“Description of Artificial Sequence: Synthetic polypeptide”
QLQLQESGPGLVKPSETLSLTCSVSGDSISSSDYYWGWIRQPPGKRLEWIGSIYYSGSTDYNPSLKSRVTISVDTSKNQLSLKLSSVTAADTAVYYCAGGDRYGPVGQWPSDYRGQGTLVTVSS

SEQ ID NO: 329 - protein, 124 aa, Artificial Sequence
  source: /note=“Description of Artificial Sequence: Synthetic polypeptide”
QLQLQESGPGLVKPSETLSLTCTVSGGSISSSDYYWGWIRQPPGKRLEWIGTIYYSGSTYYNPSLKSRVTISVDTSKNQFSLKLSSVTAADTAVYYCAGGDSYGPVGQWPSDYRGQGTLVTVSS

SEQ ID NO: 330 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTDYNPSLKSRVTISVDTSKNQFSLKLSSVTAADTAVYYCAGGYNYGPVGQWPSDYRGQGTLVTVSS

SEQ ID NO: 331 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTDYNPSLKSRVTISVDTSKNQFSLKLTSVTAADTAVYYCAGGYNYGPVGQWPSDYRGQGTLVTVSS

SEQ ID NO: 332 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYHSGSTNYNPSLKSRVTISEDTSKNQFSLKLSSVTAADTAVYYCAGGDRYGPVGQWPSDYRGQGTLVTVSS

SEQ ID NO: 333 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTYYNPSLNSRVTISVDTSKNQFSLKLSSVTAADTAVYYCAGGDTYGPVGQWPSDYRGQGTLVTVSS

SEQ ID NO: 334 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTDYNPSLKSRVTISVDTSKNQLSLKLSSVTAADTAVYYCAGGDRYGPVGQWPSDYRGQGTLVTVSS

SEQ ID NO: 335 - protein, 124 aa, Artificial Sequence
  source: /note=“Description of Artificial Sequence: Synthetic polypeptide”
QLQLQESGPGLVKPSETLSLTCTVSGDSISSSSYYWGWIRQPPGKRLEWIGSIYYSGSTNYNPSLNSRVTISVDTSRNQFSLKVSSVTAADTAVYYCAGGDTYGPVGQWPSDYRGQGTLVTVSS

SEQ ID NO: 336 - protein, 124 aa, Artificial Sequence
  source: /note=“Description of Artificial Sequence: Synthetic polypeptide”
QLQLQESGPGLVKPSETLALTCNVSGGSISSSSYYWGWIRQPPGKRLEWIGSIYYSGSTDYNPSLKSRVTISVDTSKNQFSLKLSSVTAADTAVYYCAGGYNYGPVGQWPSDYRGQGTLVTVSS

SEQ ID NO: 337 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGTIYYSGSTYYNPSLKSRVTISVDTSKNQFSLKLSSVTAADTAVYYCAGGYNYGPVGQWPSDYRGQGTLVTVSS

SEQ ID NO: 338 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGRRLEWIGSIYYSGSTHYNPSLKSRVTISVDTSKNQFSLTLSSVTAADTAVYYCAGGYNYGPVGQWPSDYRGQGTLVTVSS

SEQ ID NO: 339 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYHSGSTDYNPSLKSRVTISVDTSKNQFSLKLSSVTAADTAVYYCAGGDRYGPVGQWPSDYRGQGTLVTVSS

SEQ ID NO: 340 - protein, 124 aa, Artificial Sequence
  source: /note=“Description of Artificial Sequence: Synthetic polypeptide”
QLQLQESGPGLVKPSETLALTCNVSGGSISSSSYYWGWIRQPPGKRLEWIGSIYYSGSTDYNPSLKSRVTISVDTSKNQFSLKLTSVTAADTAVYYCAGGYNYGPVGQWPSDYRGQGTLVTVSS

SEQ ID NO: 341 - protein, 124 aa, Artificial Sequence
  source: /note=“Description of Artificial Sequence: Synthetic polypeptide”
QLQLQESGPGLVKPSETLSLTCTVSGDSISSSSYYWGWIRQPPGRRLEWIGSIYYSGSTNYNPSLKSRVTLSVDTSKNQFSLNLSSVTAADTAVYYCAGGDSYGPVGQWPSDYRGQGTLVTVSS

SEQ ID NO: 342 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYHTGSTHYNPSLKSRVTISVDTSKNQFSLTLSSVTAADTAVYYCAGGNSYGPVGQWPSDYRGQGTLVTVSS

SEQ ID NO: 343 - protein, 124 aa, Artificial Sequence
  source: /note=“Description of Artificial Sequence: Synthetic polypeptide”
QLQLQESGPGLVKPSETLSLTCTVSGGSITSSSYYWGWIRQPPGRRLEWIGSIYYSGSTYYNPSLKSRVTLSVDTSKNQFSLNLSSVTASDTAVYYCAGGDSYGPVGHWPSDYRGQGTLVTVSS

SEQ ID NO: 344 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTDYNPSLKSRVTISVDTSKNQFSLKLTSVTAADTAVYYCAGGDRYGPVGQWPSDYRGQGTLVTVSS

SEQ ID NO: 345 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGRRLEWIGSIYYSGSTDYNPSLKSRVTISVDTSKNQLSLKLSSVTAADTAVYYCAGGDRYGPVGQWPSDYRGQGTLVTVSS

SEQ ID NO: 346 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTDYNPSLKSRVTISVDTSKNQFSLTLSSVTAADTAVYYCAGGDRYGPVGQWPSDYRGQGTLVTVSS

SEQ ID NO: 347 - protein, 124 aa, Artificial Sequence
  source: /note=“Description of Artificial Sequence: Synthetic polypeptide”
QLQLQESGPGLVKPSETLSLTCTVSGGSISSSSYFWGWIRQPPGKRLEWIGTIYYSGSTNYNPSLKSRVTLSVDTSKNQFSLNLSSVTAADTAVYYCAGGDTYGPVGQWPSDYRGQGTLVTVSS

SEQ ID NO: 348 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLELIGSIYYGGSTYYNPSLKSRVTISVDTSKNQFSLKLSSVTAADTAVYYCSGGDRYGPVGQWPSDYRGQGTLVTVSS

SEQ ID NO: 349 - protein, 124 aa, Artificial Sequence
  source: /note=“Description of Artificial Sequence: Synthetic polypeptide”
QLQLQESGPGLVKPSETLSLTCTVSGDSISSSSYYWGWIRQPPGRRLEWIGSIYYSGSTNYNPSLKSRVTISVDTSKNQLSLKLSSVTAADTAVYYCAGGDRYGPVGQWPSDYRGQGTLVTVSS

SEQ ID NO: 350 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGRRLEWIGSIYYSGSTNYNPSLKSRVTLSVDTSKNQFSLNLSSVTAADTAVYYCAGGNSYGPVGQWPSDYRGQGTLVTVSS

SEQ ID NO: 351 - protein, 124 aa, Artificial Sequence
  source: /note=“Description of Artificial Sequence: Synthetic polypeptide”
QLQLQESGPGLVKPSETLALTCNVSGGSISSSSYYWGWIRQPPGKRLEWIGSIYHTGSTHYNPSLKSRVTISVDTSKNQFSLTLSSVTAADTAVYYCAGGDRYGPVGQWPSDYRGQGTLVTVSS

SEQ ID NO: 352 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLELIGSIYYGGSTYYNPSLKSRVTISVDTSKNQLSLKLSSVTAADTAIYYCAGGYNYGPVGQWPSDYRGQGTLVTVSS

SEQ ID NO: 353 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTDYNPSLRSRVTIFVDTSKNQFSLKLSSVTAADTAVYYCAGGDRYGPVGQWPSDYRGQGTLVTVSS

SEQ ID NO: 354 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGSIYYSGSTYYNPSLKSRVTISVDTSKNQFSLKLRSVTAADTAVYYCAGGDSYGPVGQWPSDYRGQGTLVTVSS

SEQ ID NO: 355 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLELIGSIYYGGSTSYNPSLKSRVTIFVDTSKNQMSLKLSSVTAADTAVYYCAGGDRYGPVGQWPSDYRGQGTLVTVSS

SEQ ID NO: 356 - protein, 124 aa, Artificial Sequence
  source: /note=“Description of Artificial Sequence: Synthetic polypeptide”
QLQLQESGPGLVKPSETLSLTCTVSGGSISSSDYYWGWIRQPPGKRPEWIGSIYYSGNTDYNPSLKSRVTISVDTSKNQFSLKLSSVTAADTAVYYCAGGDRYGPVGQWPSDYRGQGTLVTVSS

SEQ ID NO: 357 - protein, 124 aa, Artificial Sequence
  source: /note=“Description of Artificial Sequence: Synthetic polypeptide”
QLQLQESGPGLVKPSETLALTCNVSGGSISSSSYYWGWIRQPPGKRLEWIGSIYHSGSTDYNPSLKSRVTISVDTSKNQFSLKLSSVTAADTAVYYCSGGDRYGPVGQWPSDYRGQGTLVTVSS

SEQ ID NO: 358 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLEWIGNIYYSGSTYFNPSLKSRVTISVDTSKNQFSLKLSSVTAADTAVYYCAGGDTYGPVGQWPSDYRGQGTLVTVSS

SEQ ID NO: 359 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLELIGSIYYGGSTYYNPSLKSRVTISVDTSKNQFSLKLTSVTAADTAVYYCAGGYNYGPVGQWPSDYRGQGTLVTVSS

SEQ ID NO: 360 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRLELIGSIYHSGSTHYNPSLTSRVTLSVDTSKNQFSLNLSSVTAADTAVYYCAGGDSYGPVGQWPSDYRGQGTLVTVSS

SEQ ID NO: 361 - protein, 124 aa, Artificial Sequence
  source: /note=“Description of Artificial Sequence: Synthetic polypeptide”
QLQLQESGPGLVKPSETLALTCNVSGGSISSSSYYWGWIRQPPGKRLEWIGSIYYSGSTYYNPSLKSRVTISVDTSKNQFSLKLSSVTAADTAVYYCAGGDRYGPVGQWPSDYRGQGTLVTVSS

SEQ ID NO: 362 - protein, 124 aa, Artificial Sequence
  source: /note=“Description of Artificial Sequence: Synthetic polypeptide”
QLQLQESGPGLVKPSETLSLTCTVSGGSIGSSSYYWGWIRQPPGKRLEWIGSIYYSGSTYFNPSLKSRVTISVDTSKNQFSLKLSSVTAADTAVYYCSGGDRYGPVGQWPSDYRGQGTLVTVSS

SEQ ID NO: 363 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKRPEWIGSIYYSGNTYYNPSLKSRVTISVDTSKNQFSLKLSSVTAADTAVYYCAGGYNYGPVGQWPSDYRGQGTLVTVSS

SEQ ID NO: 364 - protein, 124 aa, Artificial Sequence
  source: /note=“Description of Artificial Sequence: Synthetic polypeptide”
QLQLQESGPGLVKPSETLSLTCTVSGGSISGSDYYWGWIRQPPGKGLEWIGSIYYSGRTDYNPSLKSRVTIFVDTSKNQFSLKLSSVTAADTAVYYCAGGDRYGPVGQWPSDYRGQGTLVTVSS

SEQ ID NO: 365 - protein, 124 aa, Artificial Sequence
  source: /note=“Description of Artificial Sequence: Synthetic polypeptide”
QLQLQESGPGLVKPSETLSLTCTVSGGSISSSNYYWGWIRQPPGKGLEWIGSIYYSGSTDYNPSLKSRVTLSVDTSKNQFSLNLSSVTAADTAVYYCAGGYRYGPVGQWPSDYRGQGTLVTVSS

SEQ ID NO: 366 - protein, 124 aa, Artificial Sequence
  source: /note=“Description of Artificial Sequence: Synthetic polypeptide”
QLQLQESGPGLVKPSETLSLTCTVSGGSISSSDYYWGWIRQPPGKGLEWIGSIYYSGSTYYNPSLKSRVTISVDTSKNQFSLKLSSVTAADTAVYYCAGGDRYGPVGQWPSDYRGQGTLVTVSS

SEQ ID NO: 367 - protein, 124 aa, Artificial Sequence
  source: /note=“Description of Artificial Sequence: Synthetic polypeptide”
QLQLQESGPGLVKPSETLSLNCTVSGGSISSGSYYWGWIRQPPGKGLEWIGSIYYSGSTEYNPSLKSRVTIFVDTSKNQFSLKLSSVTAADTAVYYCAGGDRYGPVGQWPSDYRGQGTLVTVSS

SEQ ID NO: 368 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKGLEWIGSIYYSGSTNYNPSLKSRVTISVDTSKNQLSLKLSSVTAADTAVYYCAGGDRYGPVGQWPSDYRGQGTLVTVSS

SEQ ID NO: 369 - protein, 124 aa, Artificial Sequence
  source: /note=“Description of Artificial Sequence: Synthetic polypeptide”
QLQLQESGPGLVKPSETLSLTCTVSGGSIGSSSYYWGWIRQPPGKGLEWIGSIYYSGSTYYKPSLKSRVTISVDTSKNQFSLKLSSVTAADTAVYYCAGGDRYGPVGQWPSDYRGQGTLVTVSS

SEQ ID NO: 370 - protein, 124 aa, Artificial Sequence
  source: /note=“Description of Artificial Sequence: Synthetic polypeptide”
QLQLQESGPGLVKPSETLSLTCAVSGDSISSSSYYWGWIRQPPGKGLEWIGSIYYSGSTNYNPSLKSRVTISVDTSKNQLSLKLSSVTAADTAVYYCAGGDRYGPVGQWPSDYRGQGTLVTVSS

SEQ ID NO: 371 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKGLEWIGSIYHSGSTDYNPSLKSRVTISVDTSKNQFSLKLTSVTAADTAVYYCAGGYNYGPVGQWPSDYRGQGTLVTVSS

SEQ ID NO: 372 - protein, 124 aa, Artificial Sequence
  source: /note=“Description of Artificial Sequence: Synthetic polypeptide”
QLQLQESGPGLVKPSETLSLTCTVSGGSISSSSYYWGWVRQPPGKGLEWIGSIYHSGSTYYNPSLKSRVTISVDTSKNQFLLKLSSVTAADTAVYYCAGGDRYGPVGQWPSDYRGQGTLVTVSS

SEQ ID NO: 373 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKGLEWIGSIYYSGSTNYNPSLKSRVTISVDTSKNQLSLKLSSVTAADTAVYYCAGGDTYGPVGQWPSDYRGQGTLVTVSS

SEQ ID NO: 374 - protein, 124 aa, Artificial Sequence
  source: /note=“Description of Artificial Sequence: Synthetic polypeptide”
QLQLQESGPGLVKPSETLSLTCTVSGGSISSSNYYWGWIRQPPGKGLEWIGSIYYSGSTDYNPSLKSRVTISVDTSKNQFSLKLTSVTAADTAVYYCAGGYNYGPVGQWPSDYRGQGTLVTVSS

SEQ ID NO: 375 - protein, 124 aa, Artificial Sequence
  source: /note=“Description of Artificial Sequence: Synthetic polypeptide”
QLQLQESGPGLVKPSETLSLTCTVSGGSISSSRYYWGWIRQPPGKGLEWIGSIYYSGSTNYNPSLKSRVTISVDTSKNQLSLKLSSVTAADTAVYYCAGGDRYGPVGQWPSDYRGQGTLVTVSS

SEQ ID NO: 376 - protein, 124 aa, Artificial Sequence
  source: /note=“Description of Artificial Sequence: Synthetic polypeptide”
QLQLQESGPGLVKPSETLYLTCTVSGGSISSSSYYWGWIRQPPGKGLEWIGSIYYSGRTDYNPSLKSRVTISVDTSKNQFSLKLSSVTAADTAVYYCAGGDSYGPVGQWPSDYRGQGTLVTVSS

SEQ ID NO: 377 - protein, 124 aa, Artificial Sequence
  source: /note=“Description of Artificial Sequence: Synthetic polypeptide”
QLQLQESGPGLVKPSETLSLTCTVSGDSISSSSYYWGWIRQPPGKELEWIGSIYHSGSTNYNPSLKSRVTISEDTSKNQFSLKLSSVTAADTAVYYCAGGDRYGPVGQWPSDYRGQGTLVTVSS

SEQ ID NO: 378 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKGLEWIGSIYYSGSTYYNPSLKSRVTISVDTSKNQFSLNLSSVTAADTAVYYCAGGDSYGPVGQWPSDYRGQGTLVTVSS

SEQ ID NO: 379 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKGLEWIGEISHIGSTNYNPSLKSRVTISVDTSKNQFSLKLSSVTAADTAVYYCAGGDRYGPVGQWPSDYRGQGTLVTVSS

SEQ ID NO: 380 - protein, 124 aa, Artificial Sequence
  source: /note=“Description of Artificial Sequence: Synthetic polypeptide”
QLQLQESGPGLVKPSETLSLTCNVSGGSISSSSYYWGWIRQPPGKGLEWIGSIYYSGSTYYNPSLKSRVTISVDTSKNQFSLNLTSVTAADTAVYYCAGGYRYGPVGQWPSDYRGQGTLVTVSS

SEQ ID NO: 381 - protein, 124 aa, Artificial Sequence
  source: /note=“Description of Artificial Sequence: Synthetic polypeptide”
QLQLQESGPGLVKPSETLSLTCTVSGGSISSRSYYWGWIRQPPGKGLEWIGSFYYSGSAYYNPSLKSRVTISVDTSKNQFSLKLTSVTAADTAVYYCAGGYNYGPVGQWPSDYRGQGTLVTVSS

SEQ ID NO: 382 - protein, 124 aa, Artificial Sequence
  source: /note=“Description of Artificial Sequence: Synthetic polypeptide”
QLQLQESGPGLVKPSETLSLTCTVSGDSISSSSYYWGWIRQPPGKRLEWIGSIYYSGSTDYNPSLKSRVTISVDTSKNQFSLKLTSVTAADTAVYYCGGGYRYGPVGQWPSDYRGQGTLVTVSS

SEQ ID NO: 383 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKGLEWIGSIYYSGDTYYNPSLKSRVTISVDTSKNQFSLKLSSVTAADTAVYYCAGGDYYGPVGQWPSDYRGQGTLVTVSS

SEQ ID NO: 384 - protein, 124 aa, Artificial Sequence
  source: /note=“Description of Artificial Sequence: Synthetic polypeptide”
QLQLQESGPGLVKPSETLSLTCTVSGDSITSTTYYWGWIRQPPGKDLEWIGSIYYSGSTDYNPSLKSRVTISVDTSKNQLSLKLSSVTAADTAVYYCAGGDRYGPVGQWPSDYRGQGTLVTVSS

SEQ ID NO: 385 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKELEWIGSIYYSGNTYYNPSLKSRVTISVDTSKSQFSLKLSSVTAADTAVYYCAGGDRYGPVGQWPSDYRGQGTLVTVSS

SEQ ID NO: 386 - protein, 124 aa, Artificial Sequence
  source: /note=“Description of Artificial Sequence: Synthetic polypeptide”
QLQLQESGPGLVKPSETLSLTCTVSGGSISSSRYYWGWIRQPPGKGLEWIGSIYYSGSTDYNPSLKSRVTIFVDTSKNQFSLKLTSVTAADTAVYYCAGGYNYGPVGQWPSDYRGQGTLVTVSS

SEQ ID NO: 387 - protein, 124 aa, Artificial Sequence
  source: /note=“Description of Artificial Sequence: Synthetic polypeptide”
QLQLQESGPGLVKPSETLSLTCTVSGDSIISSSYYWGWIRQPPGKGLEWIGSIHYSGSTYYNPSLKSRVTISVDTSKNQFSLKLTSVTAADTAVYYCAGGYRYGPVGQWPSDYRGQGTLVTVSS

SEQ ID NO: 388 - protein, 124 aa, Artificial Sequence
  source: /note=“Description of Artificial Sequence: Synthetic polypeptide”
QLQLQESGPGLVRPSETLSLTCTVSGGSISSGSHYWAWIRQPPGKGLDWIGSIYYSGSTYYNPSLKSRVTISVDTSKNQFSLKLSSVTAADTAVYYCAGGYNYGPVGQWPSDYRGQGTLVTVSS

SEQ ID NO: 389 - protein, 124 aa, Artificial Sequence
  source: /note=“Description of Artificial Sequence: Synthetic polypeptide”
QLQLQESGPGLVKPSETLSLTCTVSGGSISSSSYYWGWIRQPPGKELEWIGSIYYNGGTDYNPSLKSRVTISVDTSKNQFSLKLSSVTAADTAVYYCAGGYNYGPVGQWPSDYRGQGTLVTVSS

SEQ ID NO: 390 - protein, 124 aa, Artificial Sequence
  source: /note=“Description of Artificial Sequence: Synthetic polypeptide”
QLQLQESGPGLVKPSETLSLTCTVSGGSIGSSSYCWGWIRQPPGKRLEWIGSIYYSGSTDYNPSLKSRVTLSVDTSKNQFSLNLSSVTAADTAVYYCAGGDSYGPVGQWPSDYRGQGTLVTVSS

SEQ ID NO: 391 - protein, 124 aa, Artificial Sequence
  source: /note=“Description of Artificial Sequence: Synthetic polypeptide”
QLQLQESGPGLVKPSETLSLTCNVSGDSITDSRQYWGWIRQPPGKGLEWIGSLYYSGTTYYNPSLKSRVSISVDTSKNQFSLKLSSVTAADSAVYYCAGGYNYGPVGQWPSDYRGQGTLVTVSS

SEQ ID NO: 392 - protein, 10 aa, Artificial Sequence
  source: /note=“Description of Artificial Sequence: Synthetic peptide”
GDSISSSDYY

SEQ ID NO: 393 - protein, 10 aa, Artificial Sequence
  source: /note=“Description of Artificial Sequence: Synthetic peptide”
GGSVSSSSYY

SEQ ID NO: 394 - protein, 10 aa, Artificial Sequence
  source: /note=“Description of Artificial Sequence: Synthetic peptide”
GGSISSSSYY

SEQ ID NO: 395 - protein, 10 aa, Artificial Sequence
  source: /note=“Description of Artificial Sequence: Synthetic peptide”
GASISSSSYY

SEQ ID NO: 396 - protein, 10 aa, Artificial Sequence
  source: /note=“Description of Artificial Sequence: Synthetic peptide”
GGSISSSDYY

SEQ ID NO: 397 - protein, 10 aa, Artificial Sequence
  source: /note=“Description of Artificial Sequence: Synthetic peptide”
GDSISSSSYY

SEQ ID NO: 398 - protein, 10 aa, Artificial Sequence
  source: /note=“Description of Artificial Sequence: Synthetic peptide”
GGSITSSSYY

SEQ ID NO: 399 - protein, 10 aa, Artificial Sequence
  source: /note=“Description of Artificial Sequence: Synthetic peptide”
GGSISSSSYF

SEQ ID NO: 400 - protein, 10 aa, Artificial Sequence
  source: /note=“Description of Artificial Sequence: Synthetic peptide”
GGSIGSSSYY

SEQ ID NO: 401 - protein, 10 aa, Artificial Sequence
  source: /note=“Description of Artificial Sequence: Synthetic peptide”
GGSISGSDYY

SEQ ID NO: 402 - protein, 10 aa, Artificial Sequence
  source: /note=“Description of Artificial Sequence: Synthetic peptide”
GGSISSSNYY

SEQ ID NO: 403 - protein, 10 aa, Artificial Sequence
  source: /note=“Description of Artificial Sequence: Synthetic peptide”
GGSISSGSYY

SEQ ID NO: 404 - protein, 10 aa, Artificial Sequence
  source: /note=“Description of Artificial Sequence: Synthetic peptide”
GGSISSSRYY

SEQ ID NO: 405 - protein, 10 aa, Artificial Sequence
  source: /note=“Description of Artificial Sequence: Synthetic peptide”
GGSISSRSYY

SEQ ID NO: 406 - protein, 10 aa, Artificial Sequence
  source: /note=“Description of Artificial Sequence: Synthetic peptide”
GDSITSTTYY

SEQ ID NO: 407 - protein, 10 aa, Artificial Sequence
  source: /note=“Description of Artificial Sequence: Synthetic peptide”
GDSIISSSYY

SEQ ID NO: 408 - protein, 10 aa, Artificial Sequence
  source: /note=“Description of Artificial Sequence: Synthetic peptide”
GGSISSGSHY

SEQ ID NO: 409 - protein, 10 aa, Artificial Sequence
  source: /note=“Description of Artificial Sequence: Synthetic peptide”
GGSIGSSSYC

SEQ ID NO: 410 - protein, 10 aa, Artificial Sequence
  source: /note=“Description of Artificial Sequence: Synthetic peptide”
GDSITDSRQY

SEQ ID NO: 411 - protein, 7 aa, Artificial Sequence
  source: /note=“Description of Artificial Sequence: Synthetic peptide”
IYHSGST

SEQ ID NO: 412 - protein, 7 aa, Artificial Sequence
  source: /note=“Description of Artificial Sequence: Synthetic peptide”
IYYGGST

SEQ ID NO: 413 - protein, 7 aa, Artificial Sequence
  source: /note=“Description of Artificial Sequence: Synthetic peptide”
IYYSGST

SEQ ID NO: 414 - protein, 7 aa, Artificial Sequence
  source: /note=“Description of Artificial Sequence: Synthetic peptide”
IYHTGST

SEQ ID NO: 415 - protein, 7 aa, Artificial Sequence
  source: /note=“Description of Artificial Sequence: Synthetic peptide”
IYYSGNT

SEQ ID NO: 416 - protein, 7 aa, Artificial Sequence
  source: /note=“Description of Artificial Sequence: Synthetic peptide”
IYYSGRT

SEQ ID NO: 417 - protein, 7 aa, Artificial Sequence
  source: /note=“Description of Artificial Sequence: Synthetic peptide”
ISHIGST

SEQ ID NO: 418 - protein, 7 aa, Artificial Sequence
  source: /note=“Description of Artificial Sequence: Synthetic peptide”
FYYSGSA

SEQ ID NO: 419 - protein, 7 aa, Artificial Sequence
  source: /note=“Description of Artificial Sequence: Synthetic peptide”
IYYSGDT

SEQ ID NO: 420 - protein, 7 aa, Artificial Sequence
  source: /note=“Description of Artificial Sequence: Synthetic peptide”
IHYSGST

SEQ ID NO: 421 - protein, 7 aa, Artificial Sequence
  source: /note=“Description of Artificial Sequence: Synthetic peptide”
IYYNGGT

SEQ ID NO: 422 - protein, 7 aa, Artificial Sequence
  source: /note=“Description of Artificial Sequence: Synthetic peptide”
LYYSGTT

SEQ ID NO: 423 - protein, 16 aa, Artificial Sequence
  source: /note=“Description of Artificial Sequence: Synthetic peptide”
AGGYRYGPVGQWPSDY

SEQ ID NO: 424 - protein, 16 aa, Artificial Sequence
  source: /note=“Description of Artificial Sequence: Synthetic peptide”
AGGDTYGPVGQWPSDY

SEQ ID NO: 425 - protein, 16 aa, Artificial Sequence
  source: /note=“Description of Artificial Sequence: Synthetic peptide”
AGGYNYGPVGQWPSDY

SEQ ID NO: 426 - protein, 16 aa, Artificial Sequence
  source: /note=“Description of Artificial Sequence: Synthetic peptide”
AGGDRYGPVGQWPSDY

SEQ ID NO: 427 - protein, 16 aa, Artificial Sequence
  source: /note=“Description of Artificial Sequence: Synthetic peptide”
AGGDSYGPVGQWPSDY

SEQ ID NO: 428 - protein, 16 aa, Artificial Sequence
  source: /note=“Description of Artificial Sequence: Synthetic peptide”
AGGNSYGPVGQWPSDY

SEQ ID NO: 429 - protein, 16 aa, Artificial Sequence
  source: /note=“Description of Artificial Sequence: Synthetic peptide”
AGGDSYGPVGHWPSDY

SEQ ID NO: 430 - protein, 16 aa, Artificial Sequence
  source: /note=“Description of Artificial Sequence: Synthetic peptide”
SGGDRYGPVGQWPSDY

SEQ ID NO: 431 - protein, 16 aa, Artificial Sequence
  source: /note=“Description of Artificial Sequence: Synthetic peptide”
AGGDYYGPVGQWPSDY

SEQ ID NO: 432 - protein, 16 aa, Artificial Sequence
  source: /note=“Description of Artificial Sequence: Synthetic peptide”
GGGYRYGPVGQWPSDY

SEQ ID NO: 433 - protein, 5 aa, Artificial Sequence
  source: /note=“Description of Artificial Sequence: Synthetic peptide”
GGGGS

SEQ ID NO: 434 - protein, 10 aa, Artificial Sequence
  source: /note=“Description of Artificial Sequence: Synthetic peptide”
GGGGSGGGGS

SEQ ID NO: 435 - protein, 8 aa, Artificial Sequence
  source: /note=“Description of Artificial Sequence: Synthetic peptide”
GFTFSSYG

SEQ ID NO: 436 - protein, 10 aa, Artificial Sequence
  source: /note=“Description of Artificial Sequence: Synthetic peptide”
ADGTGPFFDY

SEQ ID NO: 437 - protein, 122 aa, Artificial Sequence
  source: /note=“Description of Artificial Sequence: Synthetic polypeptide”
EVQLLESGGGLVQPGGSLRLSCAASGFTFSSYAMSWVRQAPGKGLEWVSGISGSAGRTDYADSVKGRFTISRDNSKNTLYLQMNSLRADDTAVYHCANGKGWDGNTWYEDYRGQGTLVTVSS

SEQ ID NO: 438 - protein, 8 aa, Artificial Sequence
  source: /note=“Description of Artificial Sequence: Synthetic peptide”
ISGSGDNT

SEQ ID NO: 439 - protein, 8 aa, Artificial Sequence
  source: /note=“Description of Artificial Sequence: Synthetic peptide”
ISNSGAST

SEQ ID NO: 440 - protein, 8 aa, Artificial Sequence
  source: /note=“Description of Artificial Sequence: Synthetic peptide”
ISYDGNTI

SEQ ID NO: 441 - protein, 8 aa, Artificial Sequence
  source: /note=“Description of Artificial Sequence: Synthetic peptide”
ISSSGVST

SEQ ID NO: 442 - protein, 8 aa, Artificial Sequence
  source: /note=“Description of Artificial Sequence: Synthetic peptide”
ISDSGGRT

SEQ ID NO: 443 - protein, 8 aa, Artificial Sequence
  source: /note=“Description of Artificial Sequence: Synthetic peptide”
ISGTGDNT

SEQ ID NO: 444 - protein, 8 aa, Artificial Sequence
  source: /note=“Description of Artificial Sequence: Synthetic peptide”
ISDRGGST

SEQ ID NO: 445 - protein, 12 aa, Artificial Sequence
  source: /note=“Description of Artificial Sequence: Synthetic peptide”
ALGGSSGPFFDN

SEQ ID NO: 446 - protein, 12 aa, Artificial Sequence
  source: /note=“Description of Artificial Sequence: Synthetic peptide”
ALGGGSGPFFDN

SEQ ID NO: 447 - protein, 12 aa, Artificial Sequence
  source: /note=“Description of Artificial Sequence: Synthetic peptide”
ALGAGSGPFFDN

SEQ ID NO: 448 - protein, 12 aa, Artificial Sequence
  source: /note=“Description of Artificial Sequence: Synthetic peptide”
ALGSSSGPFFDN

SEQ ID NO: 449 - protein, 12 aa, Artificial Sequence
  source: /note=“Description of Artificial Sequence: Synthetic peptide”
ALGSGSGPFFDN

SEQ ID NO: 450 - protein, 12 aa, Artificial Sequence
  source: /note=“Description of Artificial Sequence: Synthetic peptide”
SLGSSSGPFFDN

SEQ ID NO: 451 - protein, 14 aa, Artificial Sequence
  source: /note=“Description of Artificial Sequence: Synthetic peptide”
AKRDIDSSGPFFDN

SEQ ID NO: 452 - protein, 12 aa, Artificial Sequence
  source: /note=“Description of Artificial Sequence: Synthetic peptide”
ASGGSSGPFFDN

SEQ ID NO: 453 - protein, 13 aa, Artificial Sequence
  source: /note=“Description of Artificial Sequence: Synthetic peptide”
AKDQGSSGPFFDN

SEQ ID NO: 454 - protein, 12 aa, Artificial Sequence
  source: /note=“Description of Artificial Sequence: Synthetic peptide”
TKEDSSGPFFDN

SEQ ID NO: 455 - protein, 12 aa, Artificial Sequence
  source: /note=“Description of Artificial Sequence: Synthetic peptide”
SLGGSSGPFFDN

SEQ ID NO: 456 - protein, 8 aa, Artificial Sequence
  source: /note=“Description of Artificial Sequence: Synthetic peptide”
GFTFSSYD

SEQ ID NO: 457 - protein, 8 aa, Artificial Sequence
  source: /note=“Description of Artificial Sequence: Synthetic peptide”
GFTLSSYT

SEQ ID NO: 458 - protein, 119 aa, Artificial Sequence; identical to SEQ ID NO: 272
  source: /note=“Description of Artificial Sequence: Synthetic polypeptide”

SEQ ID NO: 459 - protein, 119 aa, Artificial Sequence; identical to SEQ ID NO: 273
  source: /note=“Description of Artificial Sequence: Synthetic polypeptide”

SEQ ID NO: 460 - protein, 10 aa, Artificial Sequence
  source: /note=“Description of Artificial Sequence: Synthetic peptide”
GDSITSTTYY

(19) United States
(12) Patent Application Publication
(10) Pub. No.: US 2020/0207867 A1
(43) Pub. Date: Jul. 2, 2020

(54) HEAVY CHAIN ANTIBODIES BINDING TO ECTOENZYMES

(71) Applicant: TENEOBIO, INC., Newark, CA (US)

(72) Inventors: Starlynn Clarke, Newark, CA (US); Kevin Dang, Newark, CA (US); Shelley Force Aldred, Newark, CA (US); Nathan Trinklein, Newark, CA (US); Wim van Schooten, Newark, CA (US)

(21) Appl. No.: 16/646,970

(22) PCT Filed: Sep. 13, 2018
(86) PCT No.: PCT/US2018/050931

§ 371 Date: Mar. 12, 2020

Related U.S. Application Data
(60) Provisional application No. 62/558,147, filed on Sep. 13, 2017.

(51) Int. Cl. C07K 16/28; A61P 35/00

(52) U.S. Cl. CPC C07K 16/2896; A61P 35/00; C07K 2317/565; C07K 2317/732; C07K 2317/35; C07K 2317/734; C07K 2317/31

(57) ABSTRACT

Human heavy chain antibodies, such as UniAbs™, binding to ectoenzymes are provided along with combinations of such heavy chain antibodies and multi-specific heavy chain antibodies, targeting non-overlapping epitopes on ectoenzymes, including synergistic combinations. Methods of making such antibodies, compositions including pharmaceutical compositions comprising such antibodies, and methods directed to the treatment of disease or conditions associated with the expression of ectoenzymes are also included.

### CROSS-REFERENCE TO RELATED APPLICATIONS

This application claims priority benefit of the filing date of U.S. Provisional Patent Application Ser. No. 62/558,147, filed on Sep. 13, 2017, the disclosure of which application is herein incorporated by reference in its entirety.

### FIELD OF THE INVENTION

The present invention concerns human heavy chain antibodies (e.g. UniAb™) binding to ectoenzymes. The invention further concerns combinations of heavy chain antibodies and multi-specific heavy chain antibodies, targeting non-overlapping epitopes on ectoenzymes, including synergistic combinations of such antibodies. The invention specifically concerns anti-CD38 heavy chain antibodies, combinations, including synergistic combinations, of anti-CD38 heavy chain antibodies targeting non-overlapping epitopes on CD38, multi-specific heavy chain anti-CD38 antibodies with binding specificity to more than one non-overlapping epitope on CD38, as well as methods of making such antibodies, compositions including pharmaceutical compositions comprising such antibodies, and their various uses.

### BACKGROUND OF THE INVENTION

Ectoenzymes

Ectoenzymes are membrane proteins that have their catalytics site on the outside of the membrane in the extracellular compartment. These cell surface proteins facilitate many functions and are found on a wide variety of cells, such as immune cells, endothelial cells, and neuronal tissue cells. Ectoenzymes can be nucleotidases, cyclases, ADP-ribosyltransferases, peptidases, proteases and oxidases and include, without limitation, the following molecules: CD10, CD13, CD26, CD38, CD39, CD73, CD156b, CD156c, CD157, CD203, VAP1, ART2, and MT1-MMP.

CD38, also known as ADP-ribosyl cyclase/cyclic ADP-ribose hydrolase 1, is a single-pass type II transmembrane protein with ectoenzymatic activities. Using NAD(P) as a substrate it catalyzes the formation of several products: cyclic ADP-ribose (cADPR); ADP-ribose (ADPR); nicotinic acid adenine dinucleotide phosphate (NAADP); nicotinic acid (NA); ADP-ribose-2′-phosphate (ADPRP) (see, e.g. H. C. Lee, Mol. Med., 2006, 12: 317-323).

CD38 is expressed predominantly on immune cells including plasma cells, activated effector T cells, antigen-presenting cells, smooth muscle cells in the lung, Multiple Myeloma (MM) cells, B cell lymphoma, B cell leukemia cells, T cell lymphoma cells, breast cancer cells, myeloid derived suppressor cells, B regulatory cells, and T regulatory cells. CD38 on immune cells interacts with CD31/PECAM-1 expressed by endothelial cells and other cell lineages. This interaction promotes leukocyte proliferation, migration, T cell activation, and monocyte-derived DC maturation.

Antibodies binding to CD38 are described, for example, in Deckert et al., Clin. Cancer Res., 2014, 20(17):4574-83 and U.S. Pat. Nos. 8,153,765; 8,263,746; 8,362,211; 8,926,969; 9,187,565; 9,193,799; 9,249,226; and 9,676,869.

Daratumumab, an antibody specific for human CD38, was approved for human use in 2015 for the treatment of Multiple Myeloma (reviewed in Shallis et al., Cancer Immunol. Immunother., 2017, 66(6):697-703). Another antibody against CD38, Isatuximab (SAR650984), is in clinical trials for the treatment of Multiple Myeloma. (See, e.g., Deckert et al., Clin Cencer Res, 2014, 20(17):4574-83; Martin et al., Blood, 2015, 126:509; Martin et al., Blood, 2017, 129:3294-3303). These antibodies induce potent complement dependent cytotoxicity (CDC), antibody dependent cell-mediated cytotoxicity (ADCC), antibody dependent cellular phagocytosis (ADCP), and indirect apoptosis of tumor cells. Isatuximab also blocks the cyclase and hydrolase enzymatic activities of CD38 and induces direct apoptosis of tumor cells.

Examples of allosteric modulation of proteins by antibodies are human growth hormone, integrins, and beta-glactosidase (L. P. Roguin & L. A. Retegui, 2003, Scand. J. Immunol. 58(4):387-394). These examples show modulation of ligand-receptor interactions by single antibodies targeting different epitopes. Example of a bispecific antibody targeting two epitopes on a single molecule is against c-MET or hepatocyte growth factor receptor (HGFR) (DaSilva, J., Abstract 34: A MET x MET bispecific antibody that induces receptor degradation potently inhibits the growth of MET-addicted tumor xenografts. AACR Annual Meeting 2017; Apr. 1-5, 2017; Washington, DC).

Heavy Chain Antibodies

In a conventional IgG antibody, the association of the heavy chain and light chain is due in part to a hydrophobic interaction between the light chain constant region and the CH1 constant domain of the heavy chain There are additional residues in the heavy chain framework 2 (FR2) and framework 4 (FR4) regions that also contribute to this hydrophobic interaction between the heavy and light chains.

It is known, however, that sera of camelids (sub-order Tylopoda which includes camels, dromedaries and llamas) contain a major type of antibodies composed solely of paired H-chains (heavy-chain only antibodies or UniAbs™). The UniAbs™ of Camelidae (Camelus dromedarius, Camelus bactrianus, Lama glama, Lama guanaco, Lama alpaca and Lama vicugna) have a unique structure consisting of a single variable domain (VHH), a hinge region and two constant domains (CH2 and CH3), which are highly homologous to the CH2 and CH3 domains of classical antibodies. These UniAbs™ lack the first domain of the constant region (CH1) which is present in the genome, but is spliced out during mRNA processing. The absence of the CH1 domain explains the absence of the light chain in the UniAbs™, since this domain is the anchoring place for the constant domain of the light chain Such UniAbs™ naturally evolved to confer antigen-binding specificity and high affinity by three CDRs from conventional antibodies or fragments thereof (Muyldermans, 2001; J Biotechnol 74:277-302; Revets et al., 2005; Expert Opin Biol Ther 5:111-124). Cartilaginous fish, such as sharks, have also evolved a distinctive type of immunoglobulin, designated as IgNAR, which lacks the light polypeptide chains and is composed entirely by heavy chains. IgNAR molecules can be manipulated by molecular engineering to produce the variable domain of a single heavy chain polypeptide (vNARs) (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003)).

The ability of heavy chain-only antibodies devoid of light chain to bind antigen was established in the 1960s (Jaton et al. (1968) Biochemistry, 7, 4185-4195). Heavy chain immunoglobulin physically separated from light chain retained 80% of antigen-binding activity relative to the tetrameric antibody. Sitia et al. (1990) Cell, 60, 781-790 demonstrated that removal of the CH1 domain from a rearranged mouse μ gene results in the production of a heavy chain-only antibody, devoid of light chain, in mammalian cell culture. The antibodies produced retained VH binding specificity and effector functions.

Heavy chain antibodies with a high specificity and affinity can be generated against a variety of antigens through immunization (van der Linden, R. H., et al. Biochim. Biophys. Acta. 1431, 37-46 (1999)) and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)).

Mice in which the λ (lambda) light (L) chain locus and/or the λ and κ (kappa) L chain loci have been functionally silenced and antibodies produced by such mice are described in U.S. Pat. Nos. 7,541,513 and 8,367,888. Recombinant production of heavy chain-only antibodies in mice and rats has been reported, for example, in WO2006008548; U.S. Application Publication No. 20100122358; Nguyen et al., 2003, Immunology; 109(1), 93-101; Brüggemann et al., Crit. Rev. Immunol.; 2006, 26(5):377-90; and Zou et al., 2007, J Exp Med; 204(13): 3271-3283. The production of knockout rats via embryo microinjections of zinc-finger nucleases is described in Geurts et al., 2009, Science, 325(5939):433. Soluble heavy chain-only antibodies and transgenic rodents comprising a heterologous heavy chain locus producing such antibodies are described in U.S. Pat. Nos. 8,883,150 and 9,365,655. CAR-T structures comprising single-domain antibodies as binding (targeting) domain are described, for example, in Iri-Sofia et al., 2011, Experimental Cell Research 317:2630-2641 and Jamnani et al., 2014, Biochim Biophys Acta, 1840:378-386.

### SUMMARY OF THE INVENTION

The present invention is based, at least in part, on the finding that heavy chain antibodies, including but not limited to UniAbs™, with binding affinity to non-overlapping epitopes on an ectoenzyme have improved properties relative to antibodies binding individually to the same epitopes.

In one aspect, the invention concerns a composition comprising a combination of two or more heavy chain antibodies binding to non-overlapping epitopes on the same ectoenzyme.

In one embodiment, the ectoenzyme is selected from the group consisting of CD10, CD13, CD26, CD38, CD39, CD73, CD156b, CD156c, CD157, CD203, VAP1, ART2, and MT1-MMP.

In another embodiment, the ectoenzyme is CD38, CD39 or CD73, preferably CD38.

In a further embodiment, the heavy chain antibody is a UniAb™.

In a still further embodiment, the two or more heavy chain antibodies comprise heavy chain variable region amino acid sequences selected from the group consisting of SEQ ID NOs: 1-60, 99-149, 175-218, 247-308, and 323-391.

In an additional embodiment, the heavy chain variable region amino acid sequences are selected from the group consisting of SEQ ID NOs: 1, 99, 175, 247 and 323.

In another embodiment, the heavy chain variable region amino acid sequences are selected from the group consisting of SEQ ID NOs: 99, 175 and 323.

In yet another embodiment, the composition herein comprises a combination of a first and a second heavy chain antibody, wherein

(a) the first antibody comprises a CDR1 sequence of SEQ ID NO: 394, a CDR2 sequence of SEQ ID NO: 413, and a CDR3 sequence of SEQ ID NO: 431, and

(b) the second antibody comprises a CDR1 sequence of SEQ ID NO: 219, a CDR2 sequence of SEQ ID NO: 83 and a CDR3 sequence of SEQ ID NO: 240.

In a further embodiment, the first antibody comprises a heavy chain variable region amino acid sequence of SEQ ID NO: 323 and the second antibody comprises a heavy chain variable region amino acid sequence of SEQ ID NO: 175.

In a still further embodiment, the first and the second antibodies are IgG1.

In one embodiment, the combination of the first and second antibody is synergistic.

In a particular embodiment, the composition herein comprises a combination of UniAbs™ 309021 and 309265.

In another embodiment, the composition herein comprises a combination of a first and a second heavy chain antibody, wherein the first antibody comprises a CDR1 sequence of SEQ ID NO: 394, a CDR2 sequence of SEQ ID NO: 413, and a CDR3 sequence of SEQ ID NO: 431, and the second antibody comprises a CDR1 sequence of SEQ ID NO: 151, a CDR2 sequence of SEQ ID NO: 163 and a CDR3 sequence of SEQ ID NO: 172.

In yet another embodiment, the first antibody comprises a heavy chain variable region amino acid sequence of SEQ ID NO: 323 and the second antibody comprises a heavy chain variable region amino acid sequence of 99, where the first and the second antibodies may, for example, be IgG1 or IgG4, and may be synergistic.

In a particular embodiment, the composition comprises a combination of UniAbs™ 309021 and 309407.

In another particular embodiment, the composition comprises a UniAb™ selected from the group consisting of 309021, 309407 and 309265.

In a further aspect, the invention concerns a multi-specific heavy chain antibody having binding specificity to at least two non-overlapping epitopes on an ectoenzyme.

In one embodiment, the ectoenzyme is selected from the group consisting of CD10, CD13, CD26, CD38, CD39, CD73, CD156b, CD156c, CD157, CD203, VAP1, ART2, and MT1-MMP.

In various embodiments, the ectoenzyme is CD38, CD39 or CD73, preferably CD38.

In one embodiment, the multi-specific antibody comprises two or more heavy chain variable region amino acid sequences binding to non-ovelapping epitopes on CD38, selected from the group consisting of SEQ ID NOs: 1-60, 99-149, 175-218, 247-308, and 323-391.

In a second embodiment, the multi-specific antibody is bispecific.

In a third embodiment, the multi-specific antibody is bivalent.

In a fourth embodiment, the multi-specific antibody is tetravalent.

In a further embodiment, the multi-specific antibody is bispecific comprising (a) a first heavy chain variable region comprising a CDR1 sequence of SEQ ID NO: 394, a CDR2 sequence of SEQ ID NO: 413, and a CDR3 sequence of SEQ ID NO: 431, and (b) a second heavy chain variable region comprising a CDR1 sequence of SEQ ID NO: 219, a CDR2 sequence of SEQ ID NO: 83 and a CDR3 sequence of SEQ ID NO: 240, where the antibody may be bivalent or tetravalent.

In a still further embodiment, the multi-specific antibody comprises a first heavy chain variable region sequence of SEQ ID NO: SEQ ID NO: 323 and a second heavy chain variable region sequence of SEQ ID NO: 175, where the antibody may be bivalent or tetravalent.

In one embodiment, the multi-specific antibody herein, having the listed CDR/variable region sequences, is IgG1.

In another embodiment, the multi-specific antibody is bispecific comprising (a) a first heavy chain variable region comprising a CDR1 sequence of SEQ ID NO: 394, a CDR2 sequence of SEQ ID NO: 413, and a CDR3 sequence of SEQ ID NO: 431, and (b) a second heavy chain variable region comprising a CDR1 sequence of SEQ ID NO: 151, a CDR2 sequence of SEQ ID NO: 163 and a CDR3 sequence of SEQ ID NO: 172, where the antibody may be bivalent or tetravalent.

In yet another embodiment, the multi-specific antibody comprises a first heavy chain variable region sequence of SEQ ID NO: SEQ ID NO: 323 and a second heavy chain variable region sequence of SEQ ID NO: 99, and may be bivalent or tetravalent.

In one embodiment, the multi-specific antibody herein, having the listed CDR/variable region sequence is IgG1 or IgG4.

In another embodiment, the multi-specific antibody is a UniAb™.

In yet another embodiment, the multi-specific antibody, comprises binding specificity of one or more of UniAbs™ 309021, 309265, and 309407.

In a further embodiment, the multi-specific antibody comprises binding specificity of UniAbs™ 309021 and 309265.

In a still further embodiment, the multi-specific antibody comprises binding specificity of UniAbs™ 309021 and 309407.

In a further aspect, the invention concerns a CAR-T comprising heavy chain variable region sequences of one or more of the multi-specific antibodies herein.

In a still further aspect, the invention concerns a pharmaceutical composition comprising a composition or a multi-specific antibody or CAR-T herein.

In yet another aspect, the invention concerns a method for the treatment of a disease or condition characterized by expression of an ectoenzyme, comprising administering to a subject in need an effective amount of a pharmaceutical composition herein.

In a different aspect, the invention concerns a method for the treatment of a disease or condition characterized by expression of CD38, CD39, or CD73, comprising administering to a subject in need an effective amount of a multi-specific heavy chain antibody binding to two or more non-overlapping epitopes on CD38, CD39 or CD73.

In one embodiment, the disease or condition is characterized by expression of CD38, and may, for example, be selected from the group consisting of hematological malignancies, conditions characterized by airway hyper-responsiveness, and age-related and metabolic dysfunction characterized by nicotinamide adenine dinucleotide (NAD) decline.

In one embodiment, the hematological malignancy is selected from the group comprising multiple myeloma (MM), non-Hodgkin's lymphoma, B-cell chronic lymphocylic leukemia (CLL), B-cell acute lymphoblastic leukemia (ALL), and dT-cell ALL. The CD38 heavy chain antibodies and pharmaceutical compositions of the present invention can also be used to treat asthma and other conditions characterized by airway hyper-responsiveness, and age-related and metabolic dysfunction characterized by nicotinamide adenine dinucleotide (NAD) decline, and preferably is MM.

In a further embodiment, the multi-specific antibody used in the treatment methods herein comprises heavy chain CDR1, CDR2 and CDR3 sequences of two or more of antibodies 309021, 309265 and 309407.

In a still further embodiment, the multi-specific antibody used in the treatment methods herein comprises heavy chain variable region sequences of two or more of UniAbs™ 309021, 309265 and 309407; or heavy chain CDR1, CDR2 and CDR3 sequences of UniAbs™ 309201 and 309265, or 309021 and 309407; or heavy chain variable region sequences of UniAbs™ 309201 and 309265, or 309021 and 309407.

In another embodiment, the treatment method herein further comprises administration of one or more further agents for the treatment of MM.

In one embodiment, the further agent is selected from the group consisting of daratumumab, isatuximab, elotuzumab, and chemotherapeutic agents effective in the treatment of MM, where the chemotherapeutic agent imay, for example, be lenalidomide, dexamethasone, or bortezomib, such as lenalidomide and dexamethasone or bortezomib and dexamethasone.

In one preferred embodiment, a bispecific, bivalent heavy chain antibody having binding affinity to a first CD38 epitope and a second, non-overlapping CD38 epitope comprises a first polypeptide having binding affinity to the first CD38 epitope comprising an antigen-binding domain of a heavy-chain antibody comprising a CDR1 sequence of SEQ ID NO: 150, a CDR2 sequence of SEQ ID NO: 92, and a CDR3 sequence of SEQ ID NO: 168, at least a portion of a hinge region, and a CH domain comprising a CH2 domain and a CH3 domain, and a second polypeptide having binding affinity to the second CD38 epitope comprising an antigen-binding domain of a heavy-chain antibody comprising a CDR1 sequence of SEQ ID NO: 393, a CDR2 sequence of SEQ ID NO: 412, and a CDR3 sequence of SEQ ID NO: 424, at least a portion of a hinge region, a CH domain comprising a CH2 domain and a CH3 domain, and an asymmetric interface between the CH2 domain of the first polypeptide and the CH2 domain of the second polypeptide, and an Fc region that is a human IgG1 Fc region, a human IgG4 Fc region, a silenced human IgG1 Fc region, or a silenced human IgG4 Fc region.

In one preferred embodiment, a bispecific, tetravalent heavy chain antibody having binding affinity to a first CD38 epitope and a second, non-overlapping CD38 epitope comprises two identical polypeptides, each polypeptide comprising a first antigen-binding domain of a heavy-chain antibody having binding affinity to the first CD38 epitope, comprising a CDR1 sequence of SEQ ID NO: 150, a CDR2 sequence of SEQ ID NO: 92, and a CDR3 sequence of SEQ ID NO: 168, a second antigen-binding domain of a heavy-chain antibody having binding affinity to the second CD38 epitope, comprising a CDR1 sequence of SEQ ID NO: 393, a CDR2 sequence of SEQ ID NO: 412, and a CDR3 sequence of SEQ ID NO: 424, at least a portion of a hinge region, a CH domain comprising a CH2 domain and a CH3 domain, and an Fc region that is a human IgG1 Fc region, a human IgG4 Fc region, a silenced human IgG1 Fc region, or a silenced human IgG4 Fc region.

In one preferred embodiment, a bispecific, tetravalent heavy chain antibody having binding affinity to a first CD38 epitope and a second, non-overlapping CD38 epitope comprises a first and a second heavy chain polypeptide, wherein the first heavy chain polypeptide comprises two antigen-binding domains of a heavy-chain antibody having binding affinity to the first CD38 epitope, each antigen-binding domain comprising a CDR1 sequence of SEQ ID NO: 150, a CDR2 sequence of SEQ ID NO: 92, and a CDR3 sequence of SEQ ID NO: 168, at least a portion of a hinge region, and a CH domain comprising a CH2 domain and a CH3 domain, and an asymmetric interface between the CH2 domain of the first polypeptide and the CH2 domain of the second polypeptide, and wherein the second heavy chain polypeptide comprises two antigen-binding domains of a heavy-chain antibody having binding affinity to the second CD38 epitope, each antigen-binding domain comprising a CDR1 sequence of SEQ ID NO: 393, a CDR2 sequence of SEQ ID NO: 412, and a CDR3 sequence of SEQ ID NO: 424, at least a portion of a hinge region, and a CH domain comprising a CH2 domain and a CH3 domain, an asymmetric interface between the CH2 domain of the first polypeptide and the CH2 domain of the second polypeptide, and an Fc region that is a human IgG1 Fc region, a human IgG4 Fc region, a silenced human IgG1 Fc region, or a silenced human IgG4 Fc region.

### BRIEF DESCRIPTION OF THE DRAWINGS

FIG. 1 shows Family 1 anti-CD38 UniAb™ variable domain amino acid sequences.

FIG. 2 shows Family 1 anti-CD38 UniAb™ antibody unique CDR sequences.

FIG. 3 shows Family 1 anti-CD38 UniAb™ antibody CDR1, CDR2 and CDR3 sequences.

FIG. 4 shows Family 1 anti-CD38 UniAb™ antibody biological activities.

FIG. 5 shows Family 3 anti-CD38 UniAb™ variable domain amino acid sequences.

FIG. 6 shows Family 3 anti-CD38 UniAb™ antibody unique CDR sequences.

FIG. 7 shows Family 3 anti-CD38 UniAb™ antibody CDR1, CDR2 and CDR3 sequences.

FIG. 8 shows Family 3 anti-CD38 UniAb™ antibody biological activities.

FIG. 9 shows Family 4 anti-CD38 UniAb™ variable domain amino acid sequences.

FIG. 10 shows Family 4 anti-CD38 UniAb™ antibody unique CDR sequences.

FIG. 11 shows Family 4 anti-CD38 UniAb™ antibody CDR1, CDR2 and CDR3 sequences.

FIG. 12 shows Family 4 anti-CD38 UniAb™ antibody biological activities.

FIG. 13 shows Family 7 anti-CD38 UniAb™ variable domain amino acid sequences.

FIG. 14 shows Family 7 anti-CD38 UniAb™ antibody unique CDR sequences.

FIG. 15 shows Family 7 anti-CD38 UniAb™ antibody CDR1, CDR2 and CDR3 sequences.

FIG. 16 shows Family 7 anti-CD38 UniAb™ antibody biological activities.

FIG. 17 shows Family 9 anti-CD38 UniAb™ variable domain amino acid sequences.

FIG. 18 shows Family 9 anti-CD38 UniAb™ antibody unique CDR sequences.

FIG. 19 shows Family 9 anti-CD38 UniAb™ antibody CDR1, CDR2 and CDR3 sequences.

FIG. 20 shows Family 9 anti-CD38 UniAb™ antibody biological activities.

FIG. 21 is a schematic representation of two tetravalent, bispecific heavy chain antibodies and one bivalent, bispecific heavy chain antibody. A symmetric antibody structure is shown in panel a, and asymmetric antibodies are shown in panels b and c, expressed using knob-into-hole technology. VH domains binding non-overlapping epitopes on CD38 are shown in different shades of fill.

FIG. 22 Serum titers of UniRats™ immunized with CD38 antigens. All immunized animals have significant serum activity with human and cynomolgus (cyno) CD38 proteins in standard solid phase antigen ELISA assay.

FIG. 23 shows that UniAbs™ representing five unique heavy chain CDR3 sequence families exhibit a variety of functional behaviors with each family displaying a unique set of characteristics. A single lead VH sequence was selected from each of the five CDR3 sequence families (Clone ID Nos. 308936; 309021; 309246; 309407; and 309265) for additional functional screening in IgG1 UniAb™ format. In some assays, Daratumumab and Isatuximab were included as reference controls. Each UniAb™ was characterized for its binding to human and cyno CD38 proteins and binding to cells expressing either human or cyno CD38. In addition, the UniAbs™ were assessed for ability to inhibit the natural cyclase (enzyme) activity of CD38 as well as the ability to stimulate indirect apoptosis, direct apoptosis, ADCC and CDC on CD38-expressing mammalian cells under the appropriate assay conditions.

FIG. 24 shows CDC of different combinations UniAb™ 309407 (at a concentration of 12.5 nM) mixed with Daratumumab at different concentrations. UniDab™ 309407 did not lyse Ramos cells by CDC by itself. Daratumumab mixed with UniAb™ 309407 was more potent than Daratumumab alone. UniAb™ 309407 on a human IgG4 background also augmented CDC activity of Daratumumab. IgG4 does not bind complement. This indicates that binding of UniAb™ 309407 to CD38 modulates CDC activity of an antibody binding a non-overlapping epitope.

FIG. 25 shows complement fixation of combinations of UniAbs™ and a tetravalent bispecific UniAb™ comprising VH domains of UniAb™ ID309021 and ID309407. These two UniAbs™ and their VH domains bind 2 non-overlapping epitopes on CD38. Combining these two CD38 binders in a single tetravalent antibody (309021_309407_2XGSlink) yielded strong complement fixation and killing of tumor cells. Mixtures of UniAbs™ and tetravalent bispecific UniAb™ induced more efficacious CDC of Ramos cells compared to Daratumumab. Individual UniAbs™ did not induce CDC.

FIG. 26 shows enzyme inhibition of the cyclase activity of CD38 by bivalent and tetravalent UniAbs™. A tetravalent bispecific UniAb™ binding two non-overlapping epitopes on CD38 inhibited cyclase activity potently. Bivalent-monospecific UniAbs™ did not inhibit cyclase activity. An anti-BCMA UniAb™ was used as a negative control.

FIG. 27 shows competition between antibodies for binding to CD38. UniAbs™ from the five sequence families fall into two broad competition groups based on the ability of Daratumamab and Isatuximab to block UniAb™ binding to CD38+ cells. To identify UniAbs™ with epitopes that partially or completely overlap with epitopes for Daratumumab and Isatuximab, flow cytometry was used to measure percent of UniAb™ binding that is blocked by pre-treatment of Ramos cells with Daratumumab or Isatuximab. Increasing blocking percentages signal a higher likelihood of the two antibodies having overlapping epitopes. In this set, families F01, F04, F07 and F09 all show at least some level of blocking by both Daratumumab and Isatuximab, indicating likely binding to overlapping epitopes (placing them in competition group 1). In contrast, F03 UniAb™ (309407) binding is not blocked by pre-treatment with either Daratumumab or Isatuximab, indicating it is likely binding a distinct epitope (placing it in competition group 2).

FIG. 28 shows CDC activity on Ramos cells. UniAb™ 309021 was titrated and mixed with fixed concentration of different UniAbs™ (see legend). UniAbs™ 309407 in IgG1 and IgG4 formats showed synergy with UniAb™ 309021. UniAb™ 309265 in an IgG1 format showed synergy with UniAb™ 309021. All other UniAb™ did not synergize with UniAb™ 309021.

FIG. 29 shows CDC-mediated activity on Ramos cells by tetravalent bispecific UniAbs comprising VH domains of clone ID 321986 and clone ID 321663 compared to a mixture of bivalent monospecific mixture of these same two UniAbs

FIG. 30 shows direct tumor cell apoptosis of Ramos cells by tetravalent bispecific UniAbs comprising VH domains of clone ID 321986 and clone ID 321663.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim

The term “antibody” is used herein in the broadest sense and specifically covers monoclonal antibodies, polyclonal antibodies, monomers, dimers, multimers, multispecific antibodies (e.g., bispecific antibodies), heavy-chain only antibodies, three chain antibodies, single chain Fv, nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule, or an immunologically active portion of any of these polypeptides, i.e., a polypeptide that comprises an antigen-binding site that immunospecifically binds an antigen of a target of interest or part thereof, such targets including but not limited to, cancer cells or cells that produce autoimmune antibodies associated with an autoimmune disease. The immunoglobulins disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species. In one aspect, the immunoglobulin is of largely human origin.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010;47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001;309:657-670; Ofran et al. “Automated identification of complementarity determining regions (CDRs) reveals peculiar characteristics of CDRs and B cell epitopes.” J Immunol. 2008;181:6230-6235; Almagro “Identification of differences in the specificity-determining residues of antibodies that recognize antigens of different size: implications for the rational design of antibody repertoires.” J Mol Recognit. 2004;17:132-143; and Padlanet al. “Identification of specificity-determining residues in antibodies.” Faseb J. 1995;9:133-139., each of which is herein specifically incorporated by reference.

The terms “heavy chain-only antibody,” and “heavy chain antibody” are used interchangeably and refer, in the broadest sense, to antibodies lacking the light chain of a conventional antibody. The term specifically includes, without limitation, homodimeric antibodies comprising the VH antigen-binding domain and the CH2 and CH3 constant domains, in the absence of the CH1 domain; functional (antigen-binding) variants of such antibodies, soluble VH variants, Ig-NAR comprising a homodimer of one variable domain (V-NAR) and five C-like constant domains (C-NAR) and functional fragments thereof; and soluble single domain antibodies (sUniDabs™). In one embodiment, the heavy chain-only antibody is composed of the variable region antigen-binding domain composed of framework 1, CDR1, framework 2, CDR2, framework 3, CDR3, and framework 4. In another embodiment, the heavy chain-only antibody is composed of an antigen-binding domain, at least part of a hinge region and CH2 and CH3 domains In another embodiment, the heavy chain-only antibody is composed of an antigen-binding domain, at least part of a hinge region and a CH2 domain. In a further embodiment, the heavy chain-only antibody is composed of an antigen-binding domain, at least part of a hinge region and a CH3 domain. Heavy chain-only antibodies in which the CH2 and/or CH3 domain is truncated are also included herein. In a further embodiment the heavy chain is composed of an antigen binding domain, and at least one CH (CH1, CH2, CH3, or CH4) domain but no hinge region. The heavy chain-only antibody can be in the form of a dimer, in which two heavy chains are disulfide bonded other otherwise, covalently or non-covalently attached with each other. The heavy chain-only antibody may belong to the IgG subclass, but antibodies belonging to other subclasses, such as IgM, IgA, IgD and IgE subclass, are also included herein. In a particular embodiment, the heavy chain antibody is of the IgG1, IgG2, IgG3, or IgG4 subtype, in particular IgG1 subtype. In one embodiment, the heavy-chain antibody is of the IgG4 subtype, wherein one or more of the CH domains are modified to alter an effector function of the antibody. In one embodiment, the heavy-chain antibody is of the IgG1 subtype, wherein one or more of the CH domains are modified to alter an effector function of the antibody. Modifications of CH domains that alter effector function are further described herein.

In one embodiment, the heavy chain-only antibodies herein are used as a binding (targeting) domain of a chimeric antigen receptor (CAR). The definition specifically includes human heavy chain only antibodies produced by human immunoglobulin transgenic rats (UniRat™), called UniAb™. The variable regions (VH) of UniAb™ are called UniDabs™, and are versatile building blocks that can be linked to Fc's or serum albumin for the development of novel therapeutics with multi-specificity, increased potency and extended half-life. Since the homodimeric UniAbs™ lack a light chain and thus a VL domain, the antigen is recognized by one single domain, i.e., the variable domain of the heavy chain of a heavy-chain antibody (VH).

An “intact antibody chain” as used herein is one comprising a full length variable region and a full length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as α, δ, ϵ, γ, and μ, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called κ and λ, based on the amino acid sequences of their constant domains.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; FcγRIIB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

Variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). The human IgG1 amino acid sequence (UniProtKB No. P01857) is provided herein as SEQ ID NO: 43. The human IgG4 amino acid sequence (UniProtKB No. P01861) is provided herein as SEQ ID NO: 44. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The Fc may be in the form of having native sugar chains, increased sugar chains compared to a native form or decreased sugar chains compared to the native form, or may be in an aglycosylated or deglycosylated form. The increase, decrease, removal or other modification of the sugar chains may be achieved by methods common in the art, such as a chemical method, an enzymatic method or by expressing it in a genetically engineered production cell line. Such cell lines can include microorganisms, e.g., Pichia Pastoris, and mammalian cell lines, e.g. CHO cells, that naturally express glycosylating enzymes. Further, microorganisms or cells can be engineered to express glycosylating enzymes, or can be rendered unable to express glycosylation enzymes (See e.g., Hamilton, et al., Science, 313:1441 (2006); Kanda, et al, J. Biotechnology, 130:300 (2007); Kitagawa, et al., J. Biol. Chem., 269 (27): 17872 (1994); Ujita-Lee et al., J. Biol. Chem., 264 (23): 13848 (1989); Imai-Nishiya, et al, BMC Biotechnology 7:84 (2007); and WO 07/055916). As one example of a cell engineered to have altered sialylation activity, the alpha-2,6-sialyltransferase 1 gene has been engineered into Chinese Hamster Ovary cells and into sf9 cells. Antibodies expressed by these engineered cells are thus sialylated by the exogenous gene product. A further method for obtaining Fc molecules having a modified amount of sugar residues compared to a plurality of native molecules includes separating said plurality of molecules into glycosylated and non-glycosylated fractions, for example, using lectin affinity chromatography (See, e.g., WO 07/117505). The presence of particular glycosylation moieties has been shown to alter the function of immunoglobulins. For example, the removal of sugar chains from an Fc molecule results in a sharp decrease in binding affinity to the C1q part of the first complement component C1 and a decrease or loss in antibody-dependent cell-mediated cytotoxicity (ADCC) or complement-dependent cytotoxicity (CDC), thereby not inducing unnecessary immune responses in vivo. Additional important modifications include sialylation and fucosylation: the presence of sialic acid in IgG has been correlated with anti-inflammatory activity (See, e.g., Kaneko, et al, Science 313:760 (2006)), whereas removal of fucose from the IgG leads to enhanced ADCC activity (See, e.g., Shoj-Hosaka, et al, J. Biochem., 140:777 (2006)).

In alternative embodiments, antibodies of the invention may have an Fc sequence with enhanced effector functions, e.g., by increasing their binding capacities to FcγRIIIA and increasing ADCC activity. For example, fucose attached to the N-linked glycan at Asn-297 of Fc sterically hinders the interaction of Fc with FcγRIIIA, and removal of fucose by glyco-engineering can increase the binding to FcγRIIIA, which translates into >50-fold higher ADCC activity compared with wild type IgG1 controls. Protein engineering, through amino acid mutations in the Fc portion of IgG1, has generated multiple variants that increase the affinity of Fc binding to FcγRIIIA. Notably, the triple alanine mutant S298A/E333A/K334A displays 2-fold increase binding to FcγRIIIA and ADCC function. S239D/I332E (2X) and S239D/I332E/A330L (3X) variants have a significant increase in binding affinity to FcγRIIIA and augmentation of ADCC capacity in vitro and in vivo. Other Fc variants identified by yeast display also showed the improved binding to FcγRIIIA and enhanced tumor cell killing in mouse xenograft models. See, e.g., Liu et al. (2014) JBC 289(6):3571-90, herein specifically incorporated by reference.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, an antibody having an Fc region according to this invention can comprise an antibody with or without K447.

The term “CD38” as used herein relates to a single-pass type II transmembrane protein with ectoenzymatic activities, also known as ADP-ribosyl cyclase/cyclic ADP-ribose hydrolase 1. The term “CD38” includes a CD38 protein of any human and non-human animal species, and specifically includes human CD38 as well as CD38 of non-human mammals.

The term “human CD38” as used herein includes any variants, isoforms and species homologs of human CD38 (UniProt P28907), regardless of its source or mode of preparation. Thus, “human CD38” includes human CD38 naturally expressed by cells and CD38 expressed on cells transfected with the human CD38 gene.

The terms “anti-CD38 heavy chain-only antibody,” “CD38 heavy chain-only antibody,” “anti-CD38 heavy chain antibody” and “CD38 heavy chain antibody” are used herein interchangeably to refer to a heavy chain-only antibody as hereinabove defined, immunospecifically binding to CD38, including human CD38, as hereinabove defined. The definition includes, without limitation, human heavy chain antibodies produced by transgenic animals, such as transgenic rats or transgenic mice expressing human immunoglobulin, including UniRats™ producing human anti-CD38 UniAb™ antibodies, as hereinabove defined.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies of the invention include multi-specific antibodies. Multi-specific antibodies have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. “Multi-specific” antibodies specifically include antibodies comprising a combination of different binding entities as well as antibodies comprising more than one of the same binding entity. The terms “multi-specific antibody,” multi-specific heavy chain-only antibody,” “multi-specific heavy chain antibody,” and “multi-specific UniAb™” are used herein in the broadest sense and cover all antibodies with more than one binding specificity. The multi-specific heavy chain anti-CD38 antibodies of the present invention specifically include antibodies immunospecifically binding to more than one non-overlapping epitopes on a CD38 protein, such as a human CD38.

An “epitope” is the site on the surface of an antigen molecule to which a single antibody molecule binds. Generally an antigen has several or many different epitopes and reacts with many different antibodies. The term specifically includes linear epitopes and conformational epitopes.

“Epitope mapping” is the process of identifying the binding sites, or epitopes, of antibodies on their target antigens. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

“Polyepitopic specificity” refers to the ability to specifically bind to two or more different epitopes on the same or different target(s). As noted above, the present invention specifically includes anti-CD38 heavy chain antibodies with polyepitopic specificities, i.e. anti-CD38 heavy chain antibodies binding to two or more non-overlapping epitopes on a CD38 protein, such as a human CD38.The term “non-overlapping epitope(s)” or “non-competitive epitope(s)” of an antigen is defined herein to mean epitope(s) that are recognized by one member of a pair of antigen-specific antibodies but not the other member. Pairs of antibodies, or antigen-binding regions targeting the same antigen on a multi-specific antibody, recognizing non-overlapping epitopes do not compete for binding to that antigen and are able to bind that antigen simultaneously.

An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in all number of different formats, using either labeled antigen or labeled antibody. Usually, the antigen is immobilized on a 96-well plate, and the ability of unlabeled antibodies to block the binding of labeled antibodies is measured using radioactive or enzyme labels.

The term “valent” as used herein refers to a specified number of binding sites in an antibody molecule.

A “multi-valent” antibody has two or more binding sites. Thus, the terms “bivalent”, “trivalent”, and “tetravalent” refers to the presence of two binding sites, three binding sites, and four binding sites, respectively. Thus, a bispecific antibody according to the invention is at least bivalent and may be trivalent, tetravalent, or otherwise multi-valent.

A large variety of methods and protein configurations are known and used for the preparation of bispecific monoclonal antibodies (BsMAB), tri-specific antibodies, and the like.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9. Non-limiting examples of such linkers include GGGGS (SEQ ID NO: 433) (n=1) and GGGGSGGGGS (SEQ ID NO: 434) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety.

The term “bispecific three-chain antibody like molecule” or “TCA” is used herein to refer to antibody-like molecules comprising, consisting essentially of, or consisting of three polypeptide subunits, two of which comprise, consist essentially of, or consist of one heavy and one light chain of a monoclonal antibody, or functional antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy chain only antibody comprising an Fc portion comprising CH2 and/or CH3 and/or CH4 domains, in the absence of a CH1 domain, and an antigen binding domain that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and J_{H} gene segments, or J_{L} gene segments. The variable region may be encoded by rearranged V_{H}DJ_{H}, V_{L}DJ_{H}, V_{H}J_{L}, or V_{L}J_{L} gene segments. A TCA protein makes use of a heavy chain-only antibody as hereinabove defined.

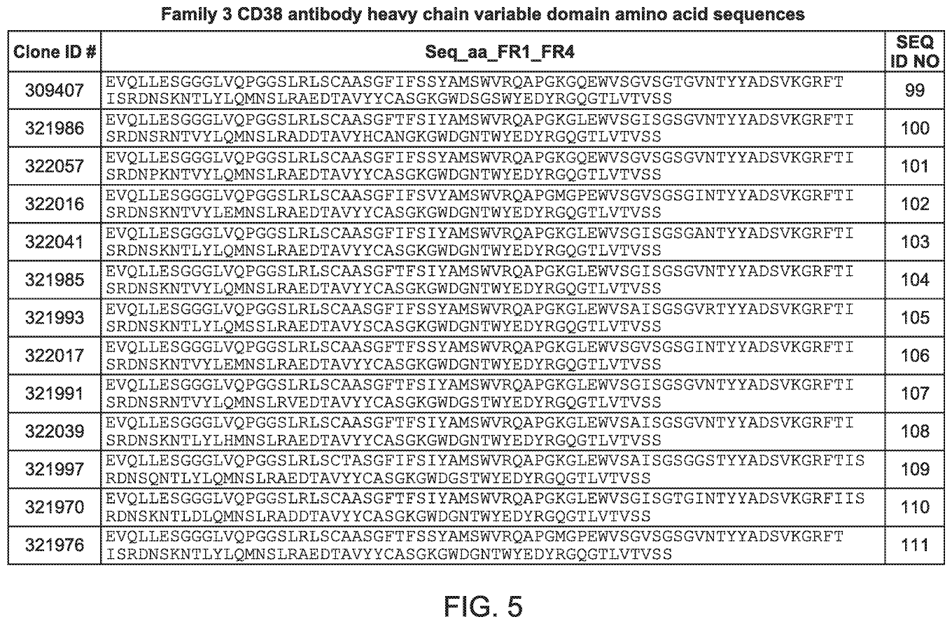

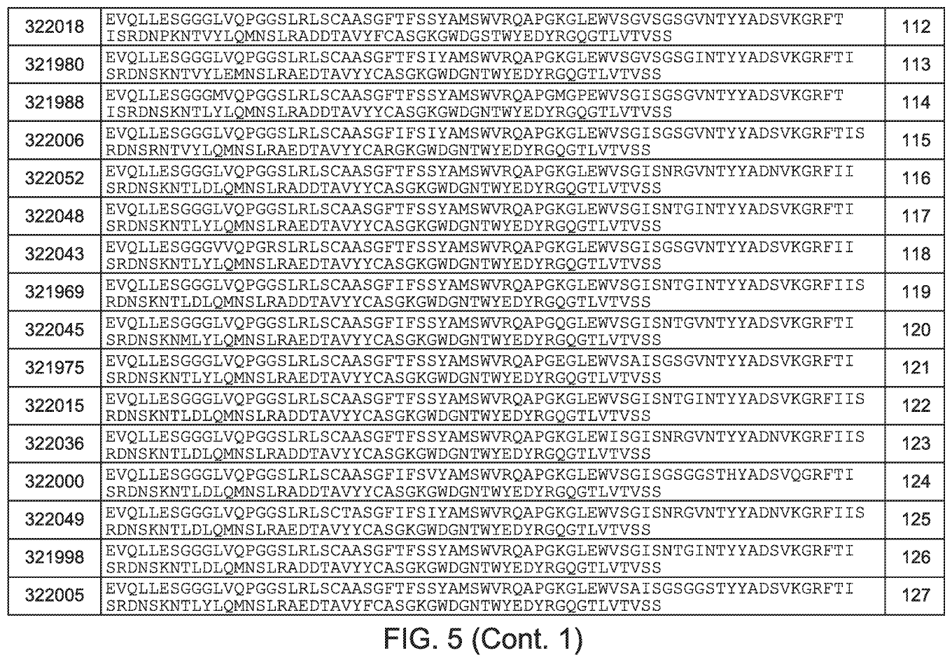

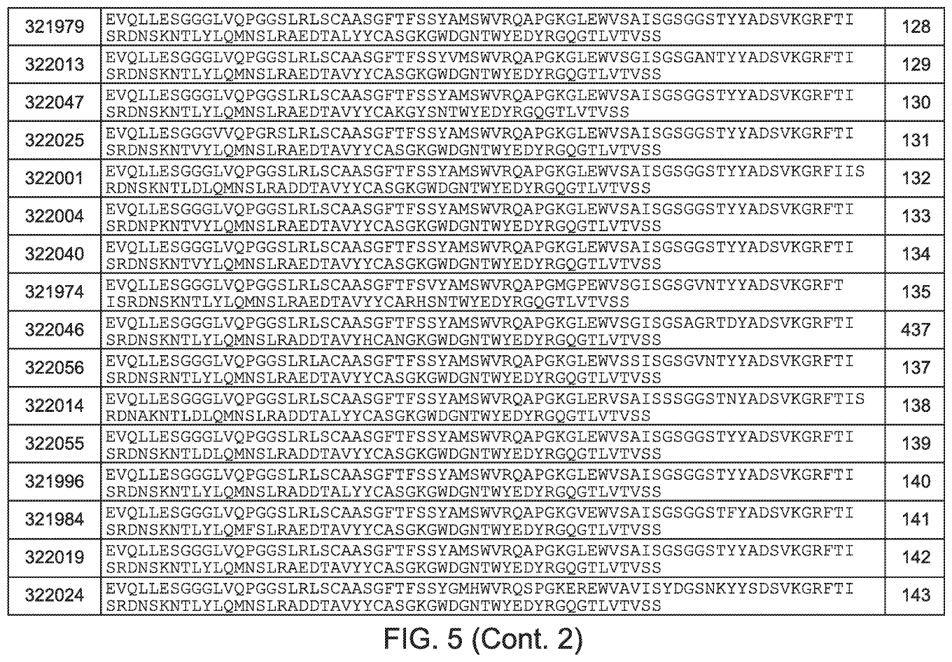

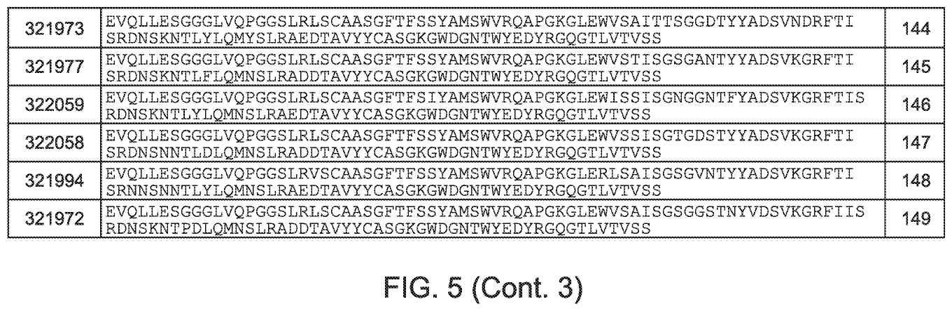

The term “chimeric antigen receptor” or “CAR” is used herein in the broadest sense to refer to an engineered receptor, which grafts a desired binding specificity (e.g. the antigen-binding region of a monoclonal antibody or other ligand) to membrane-spanning and intracellular-signaling domains. Typically, the receptor is used to graft the specificity of a monoclonal antibody onto a T cell to create a chimeric antigen receptors (CAR). (J Natl Cancer Inst, 2015; 108(7):dvj439; and Jackson et al., Nature Reviews Clinical Oncology, 2016; 13:370-383.) A representative CAR-T construct comprising a human VH extracellular binding domain is shown in FIG. 5 (panel B) in comparison to an scFv CAR-T construct (panel A).

The term “human antibody” is used herein to include antibodies having variable and constant regions derived from human germline immunoglobulin sequences. The human antibodies herein may include amino acid residues not encoded by human germline immunoglobulin sequences, e.g. mutations introduced by random or site-specific mutagenesis in vitro or by somatic mutation in vivo. The term “human antibody” specifically includes heavy chain only antibodies having human heavy chain variable region sequences, produced by transgenic animals, such as transgenic rats or mice, in particular UniAb™ produced by UniRats™, as defined above.

By a “chimeric antibody” or a “chimeric immunoglobulin” is meant an immunoglobulin molecule comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the invention that have been engineered to produce such chimeric antibodies.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes andmacrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T cell receptors or FcRs and perform effector functions. Preferably, the cells express at least FcγRIII and perform ADCC effector function. Examples of human leukocytes which mediate ADCC include natural killer (NK) cells, monocytes, cytotoxic T cells and neutrophils; with NK cells being preferred. The effector cells may be isolated from a native source thereof, e.g. from blood or PBMCs as described herein.

The term “immune cell” is used herein in the broadest sense, including, without limitation, cells of myeloid or lymphoid origin, for instance lymphocytes (such as B cells and T cells including cytolytic T cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Binding affinity ” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, Calif.) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12(8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “B-cell neoplasms” or “mature B-cell neoplasms” in the context of the present invention include small lymphocytic lymphoma, B-cell prolymphocytic lymphoma, B-cell chronic lymphocytic leukemia, mantle cell lymphoma, Burkitt's lymphoma, follicular lymphoma, diffuse large B-cell lymphoma, multiple myeloma, lymphoplasmacytic lymphoma, splenic marginal zone lymphoma, plasma cell neoplasms, such as plasma cell myeloma, plasmacytoma, monoclonal immunoglobulin deposition disease, heavy chain disease, MALT lymphoma, nodal marginal B cell lymphoma, intravascular large B cell lymphoma, primary effusion lymphoma, lymphomatoid granulomatosis, non-Hodgkins lymphoma, Hodgkins lymphoma, hairy cell leukemia, primary effusion lymphoma and AIDS-related non-Hodgkins lymphoma.

The terms “subject,” “individual,” and “patient” are used interchangeably herein to refer to a mammal being assessed for treatment and/or being treated. In an embodiment, the mammal is a human The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g. mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomeriation), clipping/hydrolysis/fragmentation (e.g., hinge region fragmentation), succinimide formation, unpaired cysteine(s), N-terminal extension, C-terminal processing, glycosylation differences, etc.

### II. Detailed Description

The invention is based, at least in part, on the finding that combinations of heavy chain antibodies binding non-overlapping epitopes on ectoenzymes work synergistically to lyse tumor cells and/or inhibit enzymatic activity of the target ectoenzyme Similarly, multi-specific, e.g. bispecific heavy chain antibodies having binding specificity to at least two non-overlapping epitopes on ectoenzymes act synergistically to kill tumor cells and/or inhibit the enzymatic activity of the target ectoenzyme.

### Ectoenzymes

Ectoenzymes are a diverse group of membrane proteins having catalytic sites outside the plasma membrane. Many of the ectoenzymes are found on leukocytes and endothelial cells, where they play multiple biological roles. Apart from the extracellular catalytic activity that is common to all, ectoenzymes are a diverse class of molecules that are involved in very different types of enzymatic reactions. Different ectoenzymes can modulate each step of leukocyte-endothelial contacts, as well as subsequent cell migration in tissues. Ectoenzymes include, without limitation, CD38, CD10, CD13, CD26, CD39, CD73, CD156b, CD156c, CD157, CD203, VAP1, ART2, and MT1-MMP.

The ectoenzyme CD38 belongs to the family of nucleotide-metabolizing enzymes which, in addition to recycling nucleotides generate compounds that control cellular homeostasis and metabolism. The catalytic activity of CD38 is required for various physiological processes, including insulin secretion, muscarinic Ca²⁺ signaling in pancreatic acinar cells, neutrophil chemotaxis, dendritic cell trafficking, oxytoxin secretion, an in the development of diet-induced obesity. See, Vaisitti et al., Laeukemia, 2015, 29: 356-368, and the references cited therein. CD38 is expressed in a variety of malignancies, including chronic lymphocytic leukemia (CLL). CD38 has been shown to identify a particularly aggressive form of CLL, and is considered a negative prognostic marker, predicting a shorter overall survival of patients with this aggressive variant of CLL. See, Malavasi et al., 2011, Blood, 118:3470-3478, and Vaisitti, 2015, supra.

### Preparation of Anti-Ectoenzyme Heavy Chain Antibodies

The heavy chain antibodies of the present invention can be prepared by methods known in the art. In a preferred embodiment, the heavy chain antibodies herein are produced by transgenic animals, including transgenic mice and rats, preferably rats, in which the endogenous immunoglobulin genes are knocked out or disabled. In a preferred embodiment, the heavy chain antibodies herein are produced in UniRat™. UniRat™ have their endogenous immunoglobulin genes silenced and use a human immunoglobulin heavy-chain translocus to express a diverse, naturally optimized repertoire of fully human HCAbs. While endogenous immunoglobulin loci in rats can be knocked out or silenced using a variety technologies, in UniRat™ the zinc-finger (endo)nuclease (ZNF) technology was used to inactivate the endogenous rat heavy chain J-locus, light chain Cκ locus and light chain Cλ locus. ZNF constructs for microinjection into oocytes can produce IgH and IgL knock out (KO) lines. For details see, e.g. Geurts et al., 2009, Science 325:433 Characterization of Ig heavy chain knockout rats has been reported by Menoret et al., 2010, Eur. J. Immunol. 40:2932-2941. Advantages of the ZNF technology are that non-homologous end joining to silence a gene or locus via deletions up to several kb can also provide a target site for homologous integration (Cui et al., 2011, Nat Biotechnol 29:64-67). Human heavy chain antibodies produced in UniRat™ are called UniAbs™ can bind epitopes that cannot be attacked with conventional antibodies. Their high specificity, affinity, and small size make them ideal for mono- and poly-specific applications.

In addition to UniAbs™, specifically included are heavy chain only antibodies lacking the camelid VHH framework and mutations, and their functional VH regions. Such heavy chain only antibodies can, for example, be produced in transgenic rats or mice which comprise fully human heavy chain-only gene loci as described, e.g. in WO2006/008548, but other transgenic mammals, such as rabbit, guinea pig, rat can also be used, rats and mice being preferred. Heavy chain only antibodies, including their VHH or VH functional fragments, can also be produced by recombinant DNA technology, by expression of the encoding nucleic acid in a suitable eukaryotic or prokaryotic host, including E. coli or yeast.

Domains of heavy chain only antibodies combine advantages of antibodies and small molecule drugs: can be mono- or multi-valent; have low toxicity; and are cost-effective to manufacture. Due to their small size, these domains are easy to administer, including oral or topical administration, are characterized by high stability, including gastrointestinal stability; and their half-life can be tailored to the desired use or indication. In addition, VH and VHH domains of HCAbs can be manufactured in a cost effective manner.

In a particular embodiment, the heavy chain antibodies of the present invention, including UniAbs™, have the native amino acid residue at the first position of the FR4 region (amino acid position 101 according to the Kabat numbering system), substituted by another amino acid residue, which is capable of disrupting a surface-exposed hydrophobic patch comprising or associated with the native amino acid residue at that position. Such hydrophobic patches are normally buried in the interface with the antibody light chain constant region but become surface exposed in HCAbs and are, at least partially, for the unwanted aggregation and light chain association of HCAbs. The substituted amino acid residue preferably is charged, and more preferably is positively charged, such as lysine (Lys, K), arginine (Arg, R) or histidine (His, H), preferably arginine (R). In a preferred embodiment the heavy chain only antibodies derived from the transgenic animals contain a Trp to Arg mutation at position 101. The resultant HCAbs preferably have high antigen-binding affinity and solubility under physiological conditions in the absence of aggregation.

In certain embodiments, the anti-ectoenzyme heavy chain antibodies bind CD38. In a preferred embodiment, the anti-CD38 heavy chain only antibodies are UniAbs™.

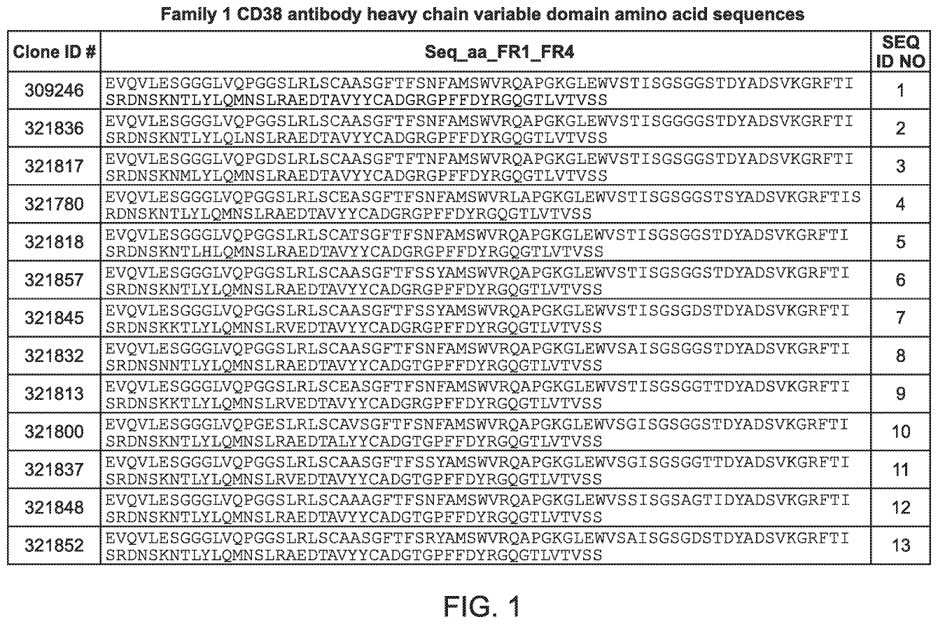

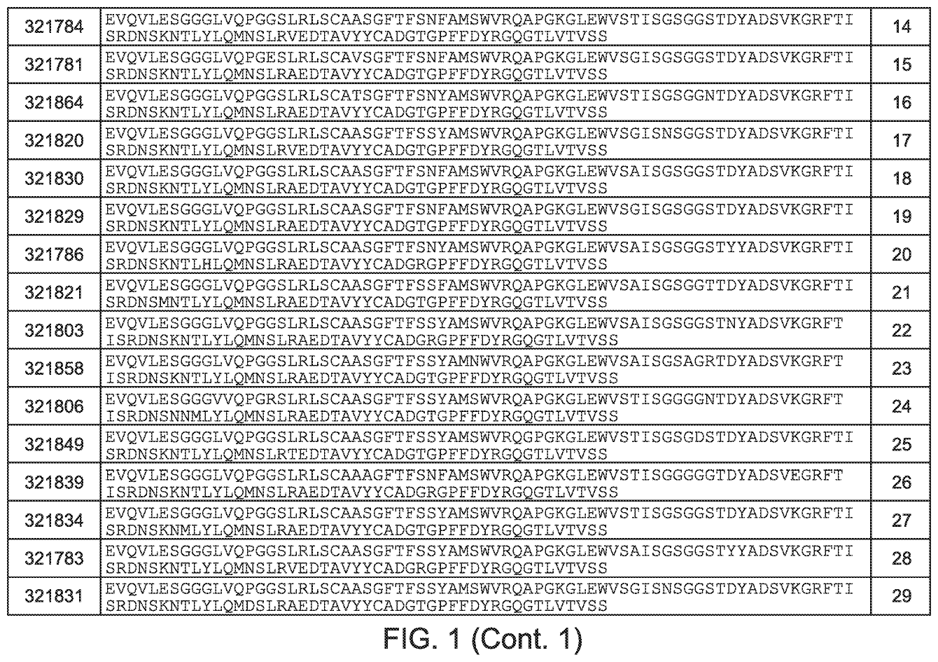

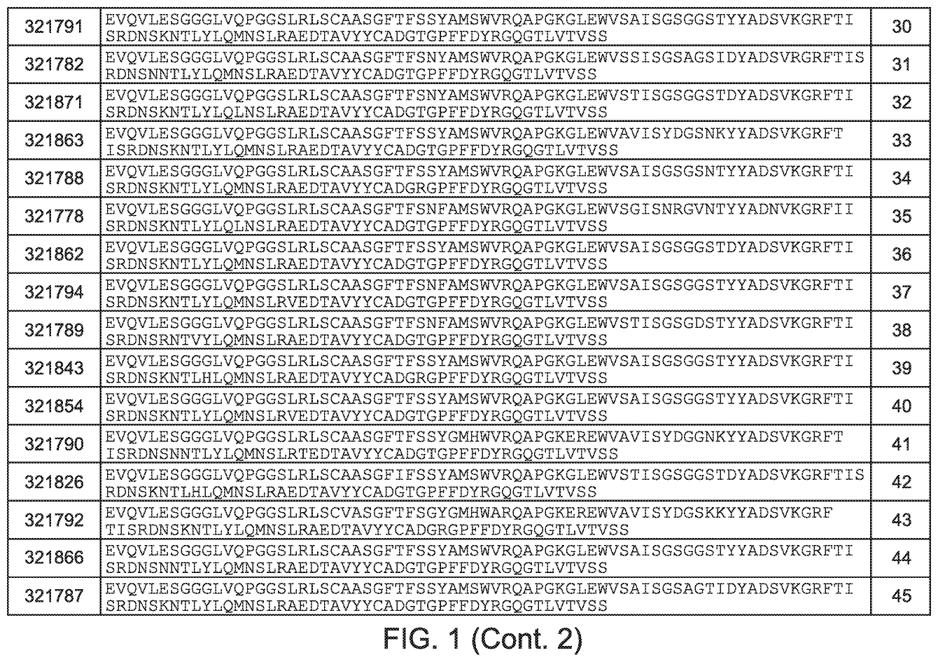

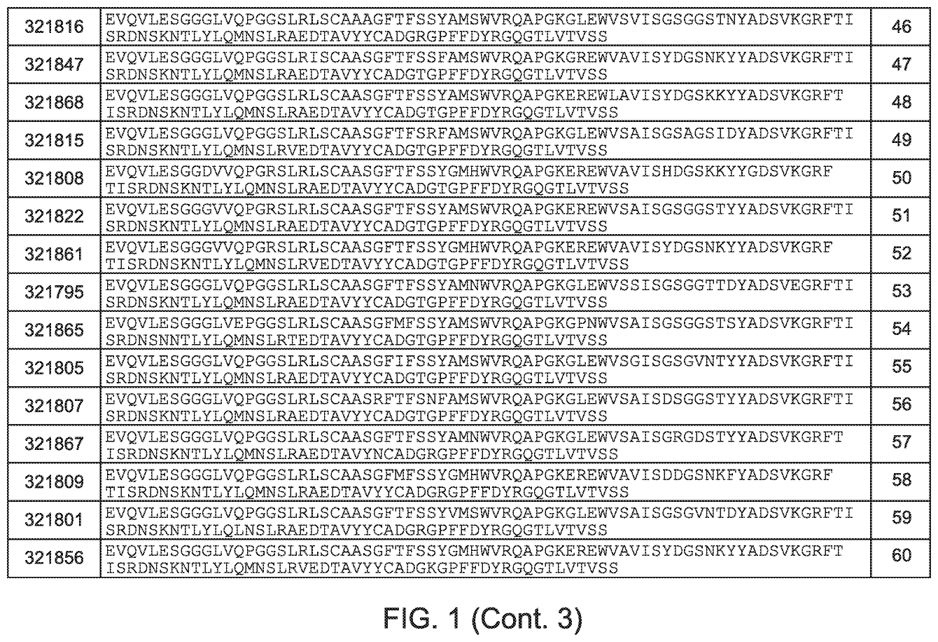

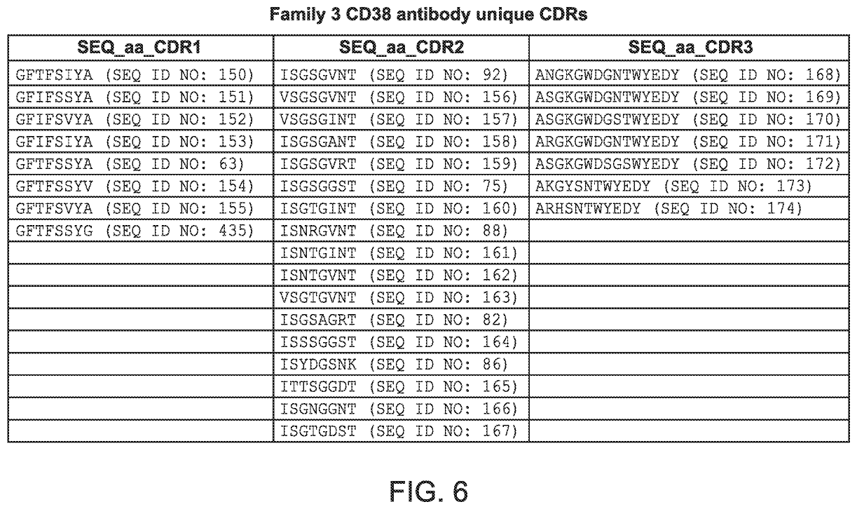

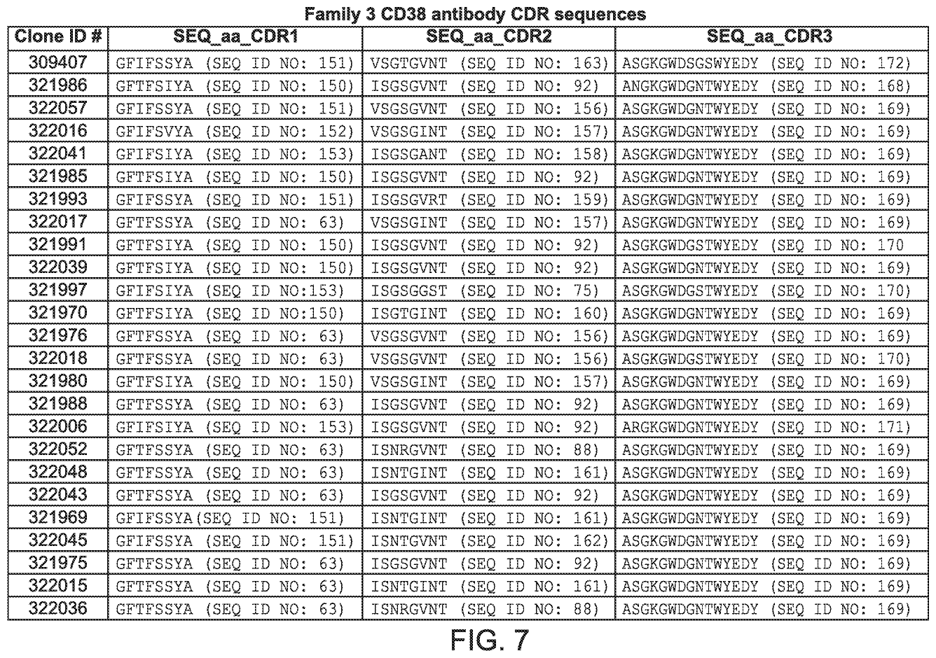

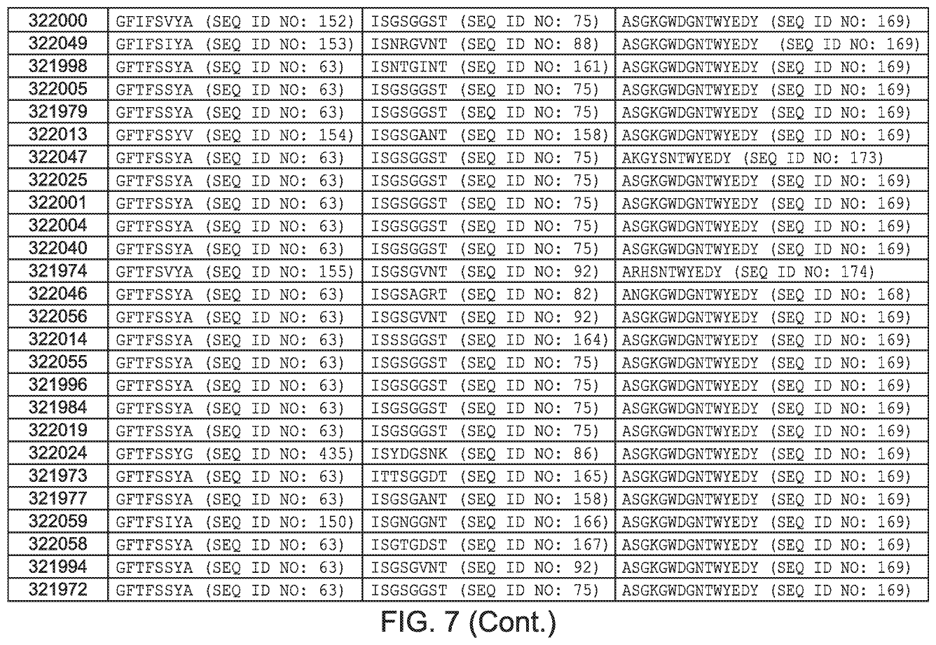

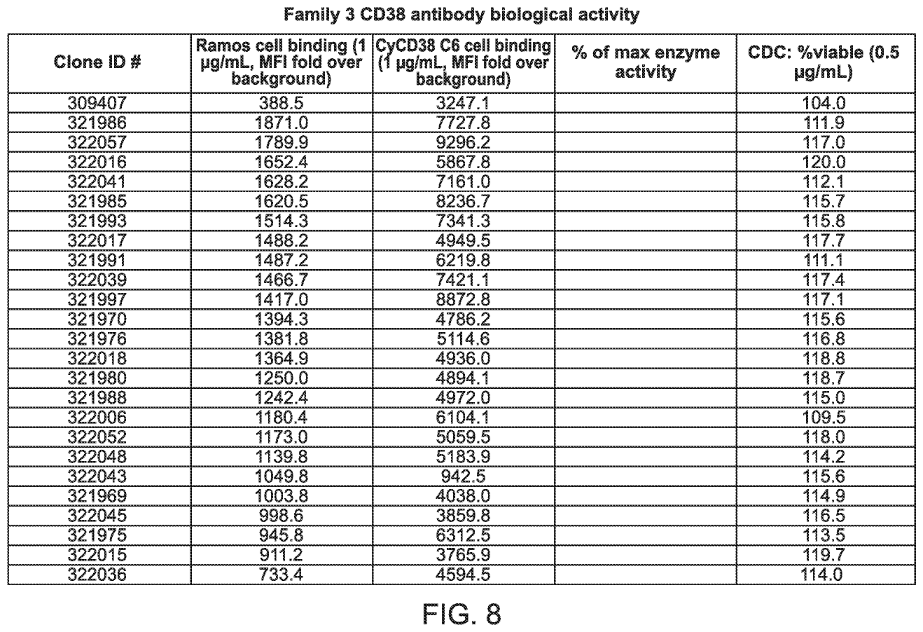

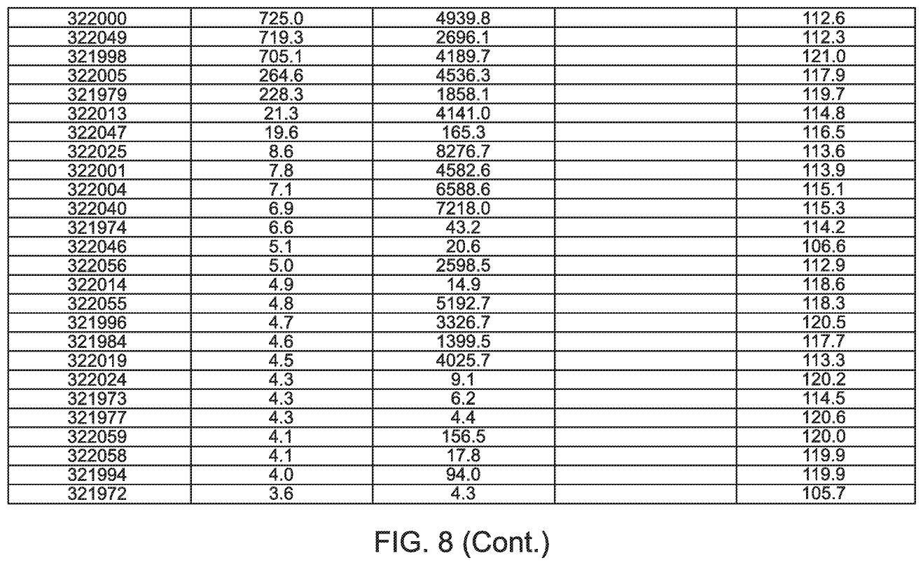

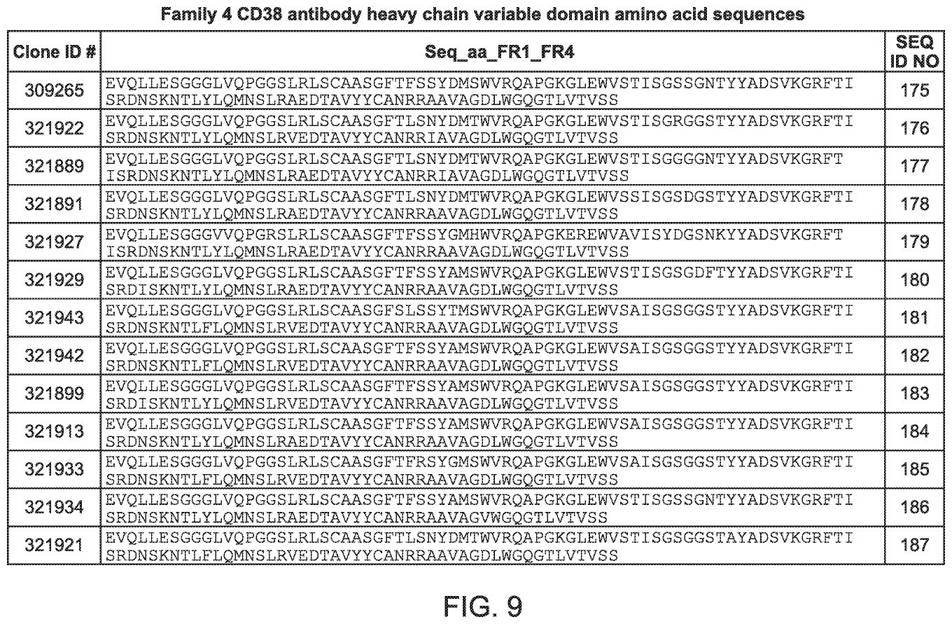

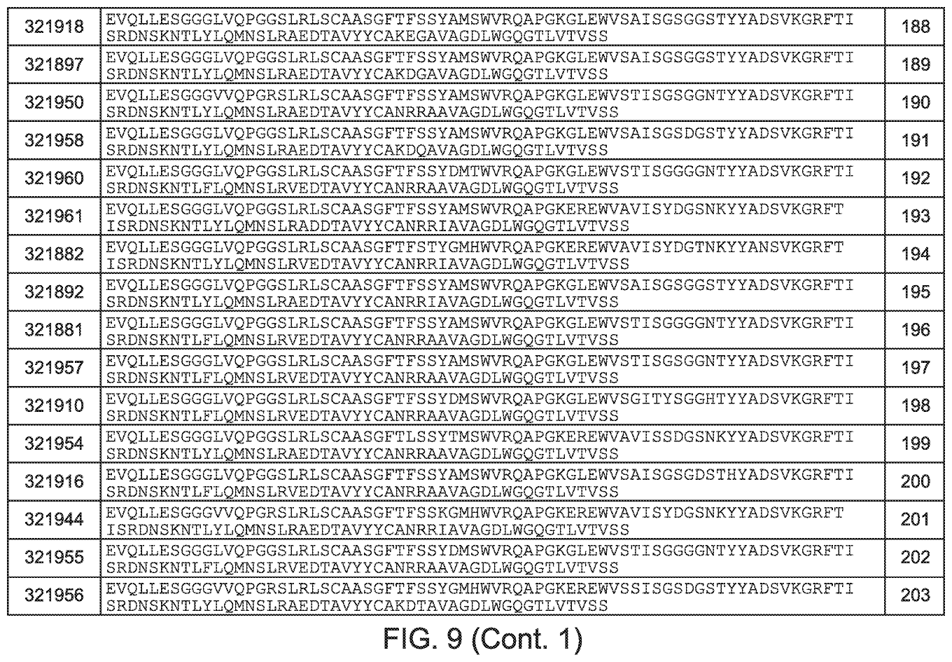

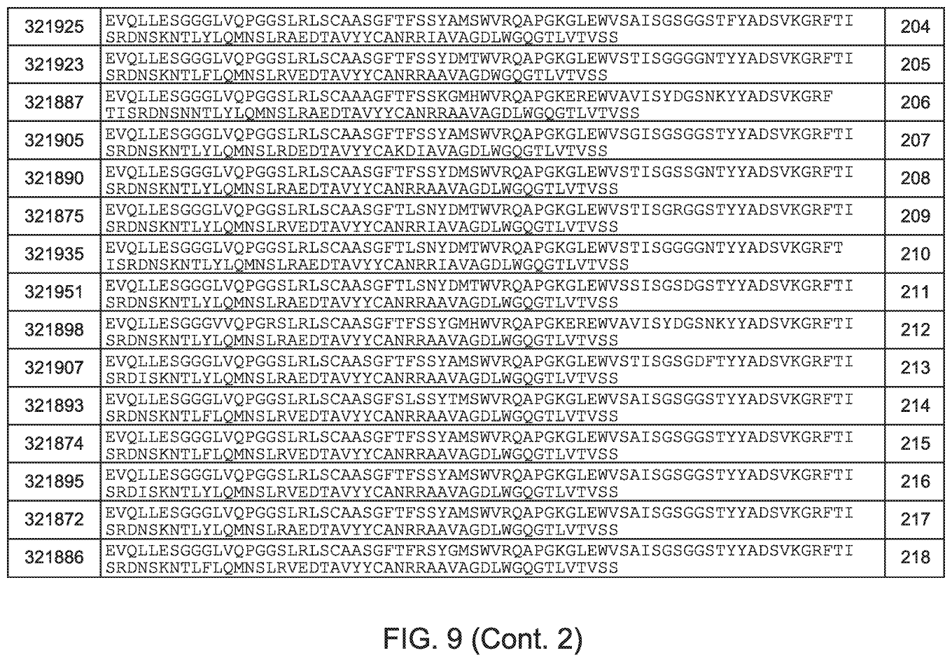

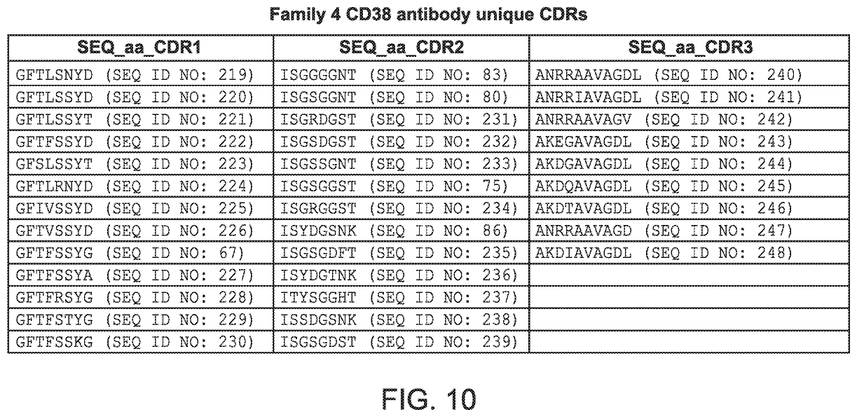

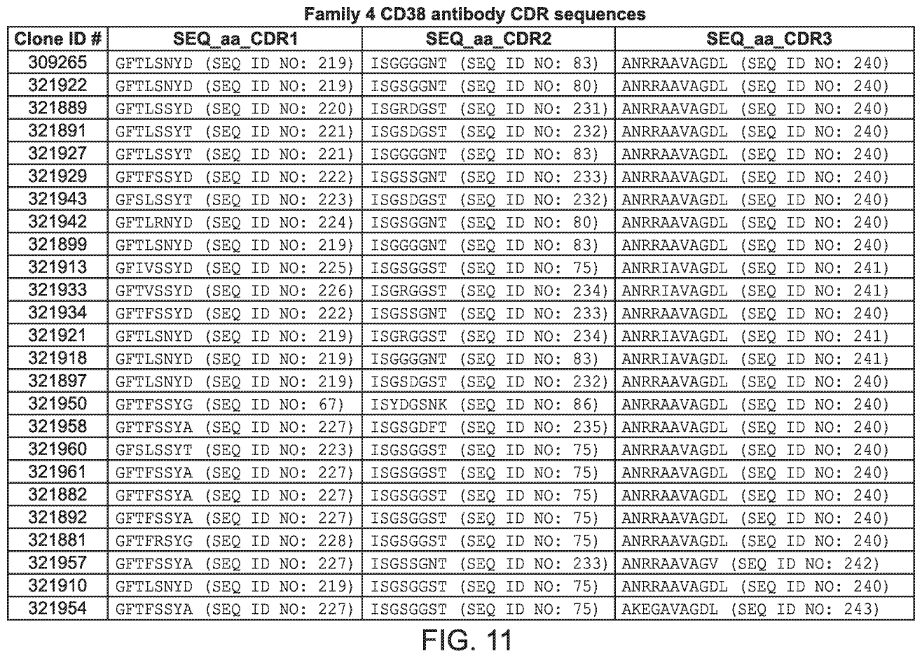

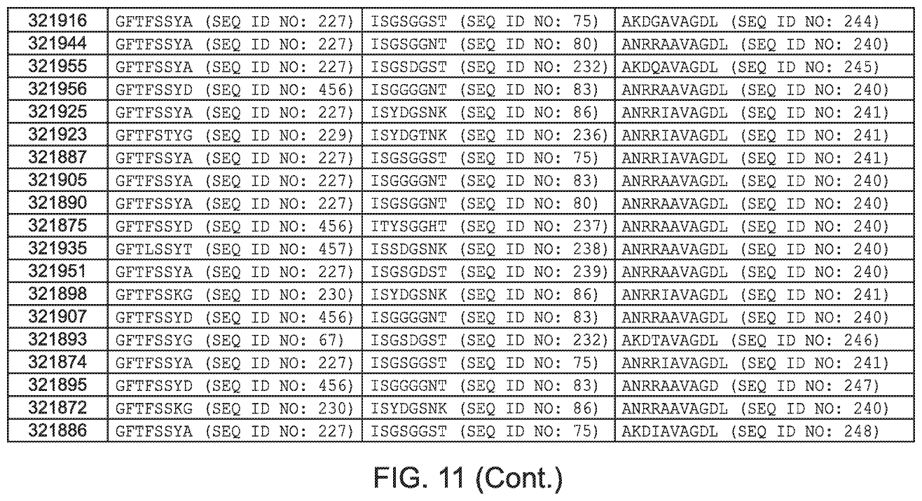

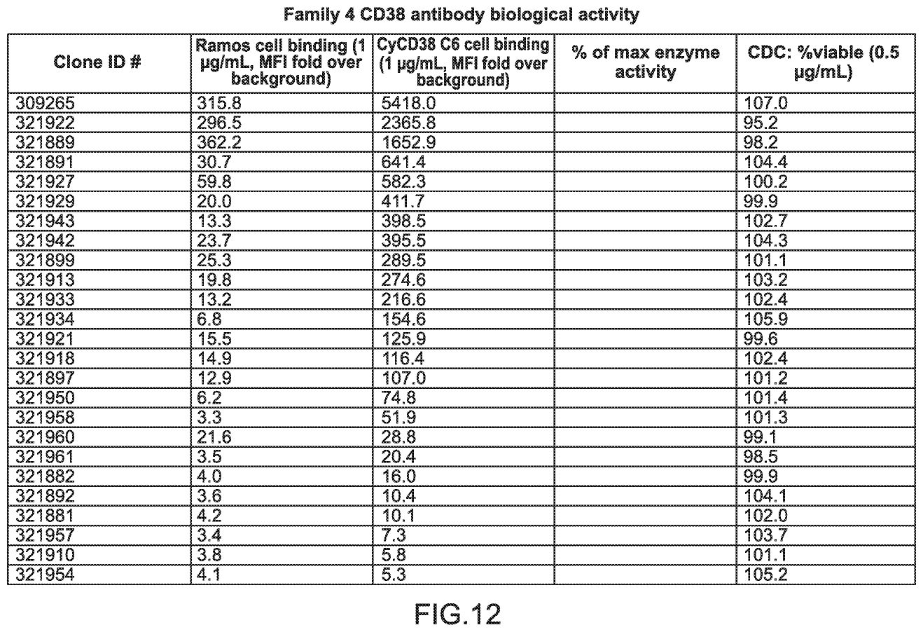

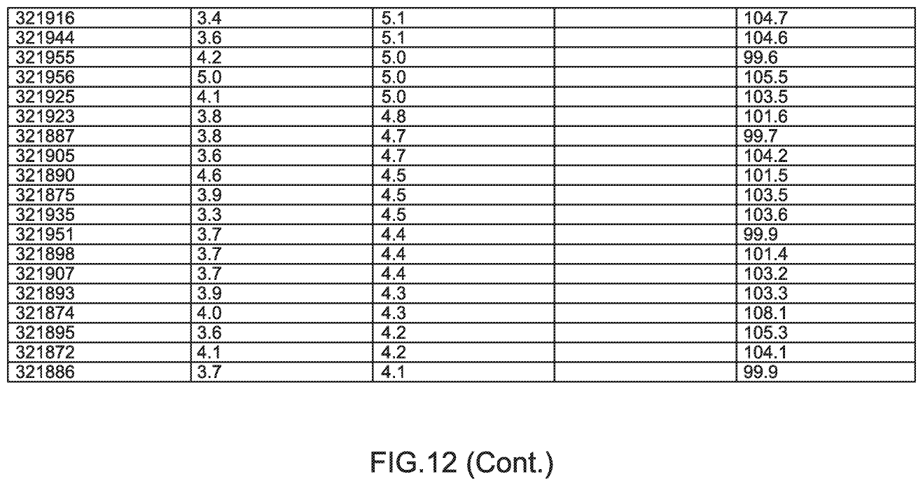

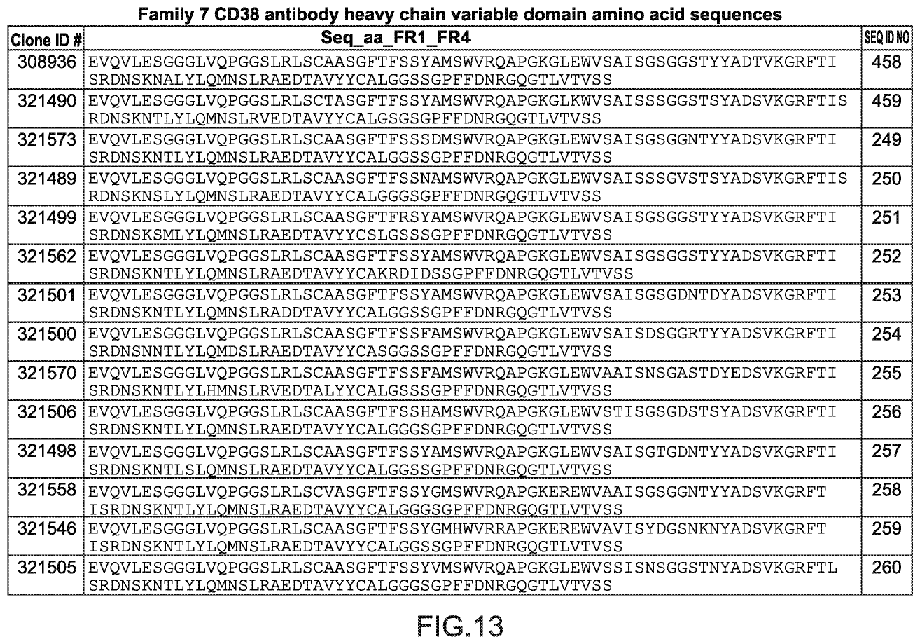

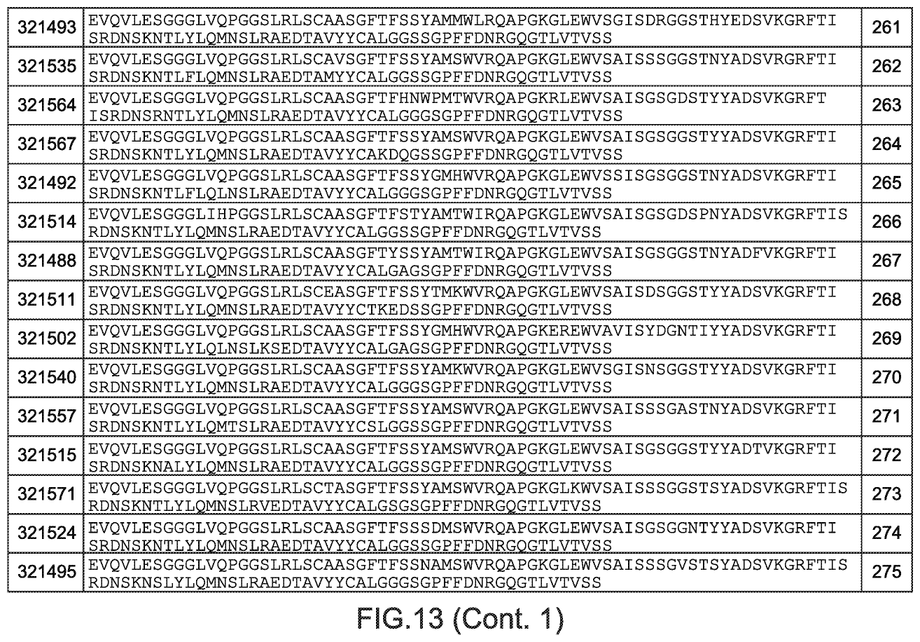

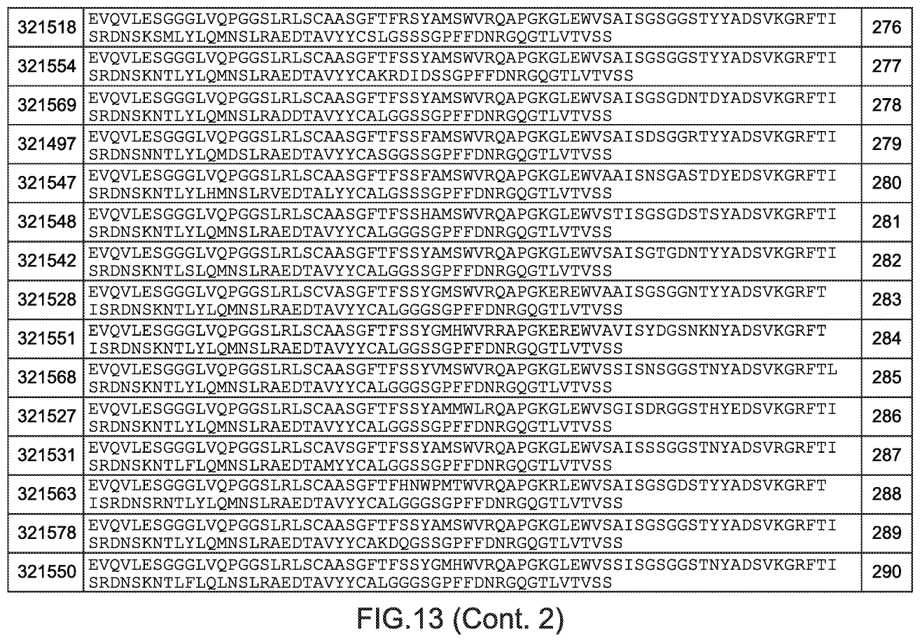

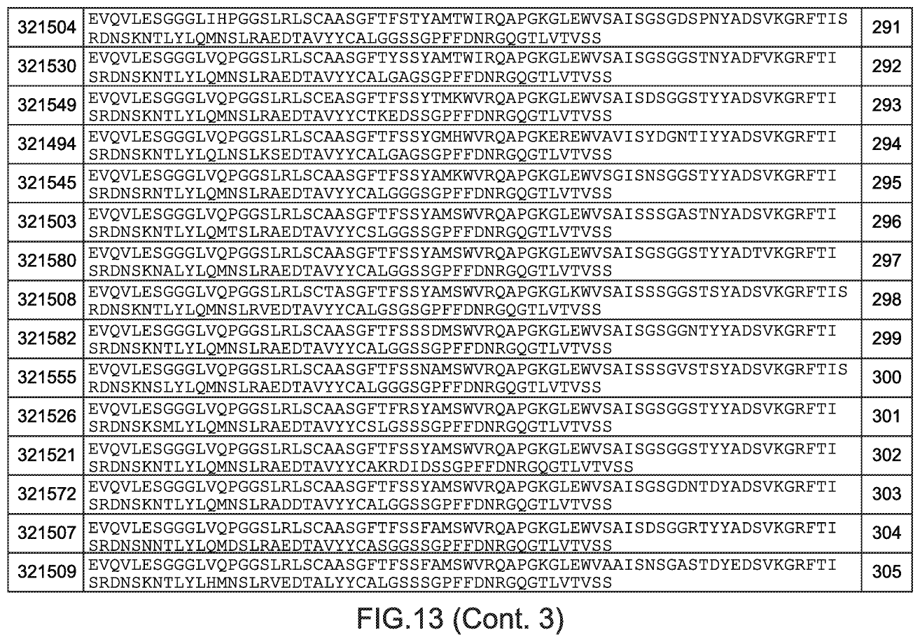

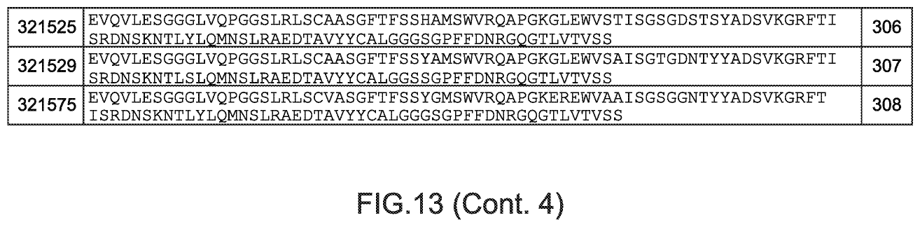

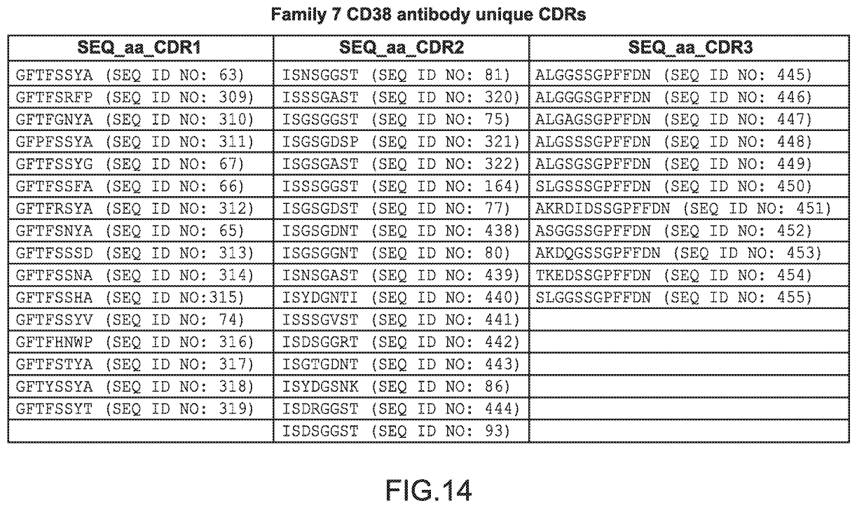

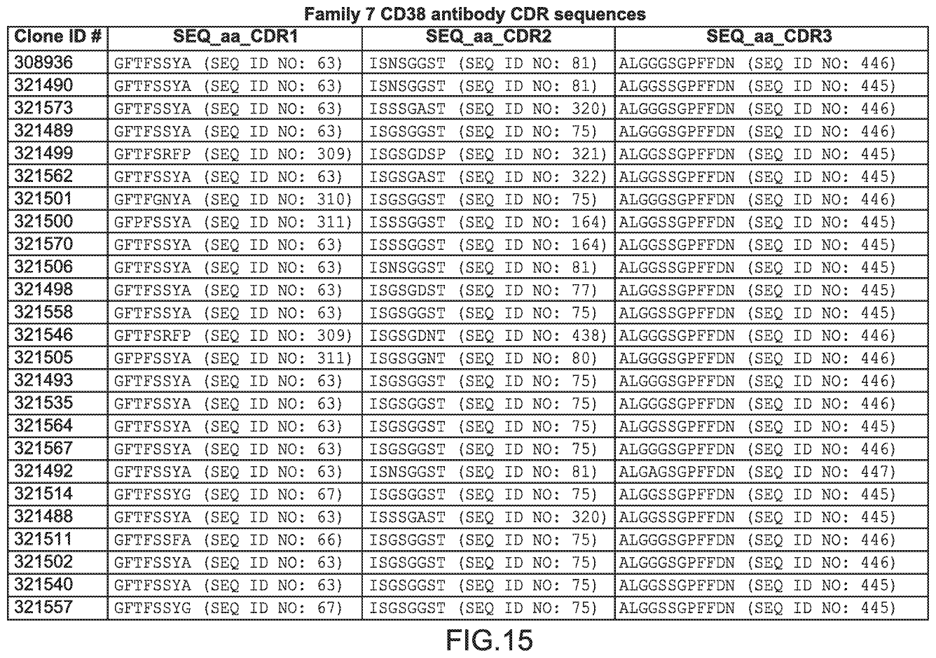

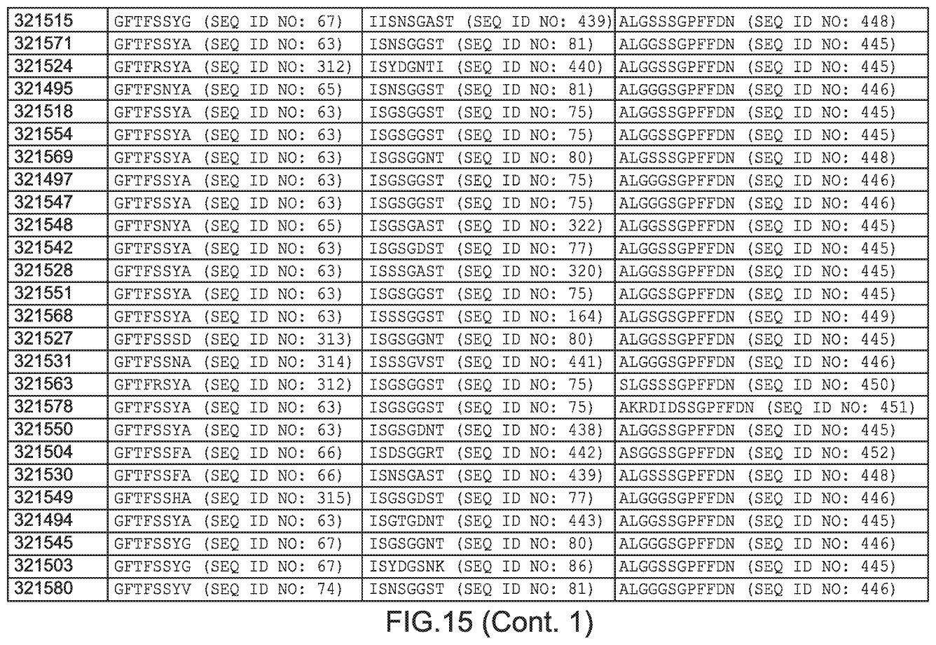

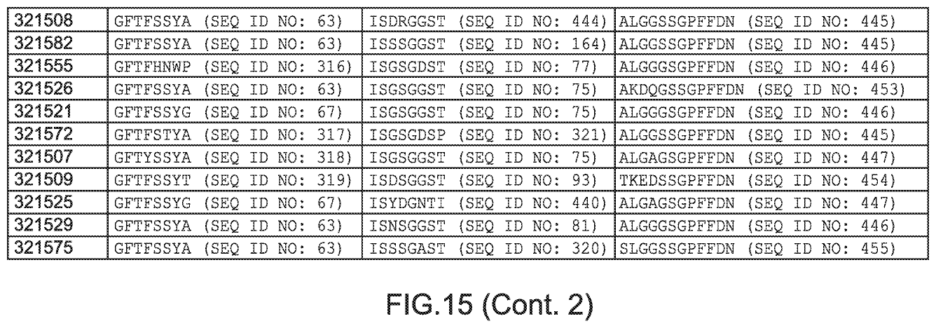

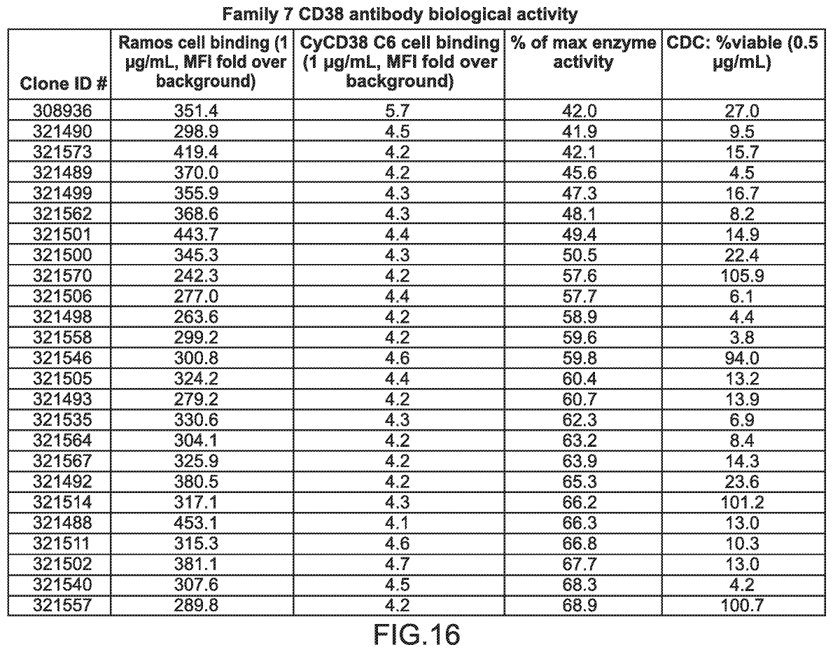

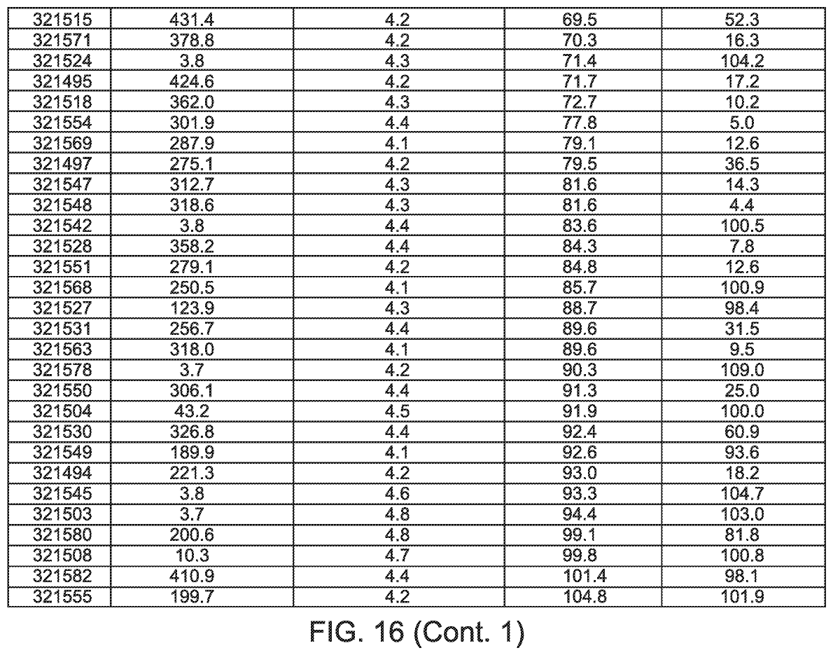

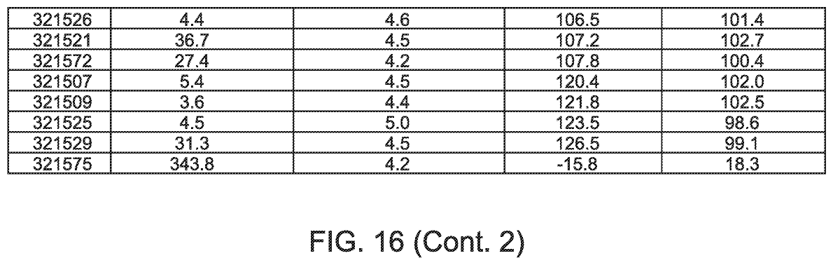

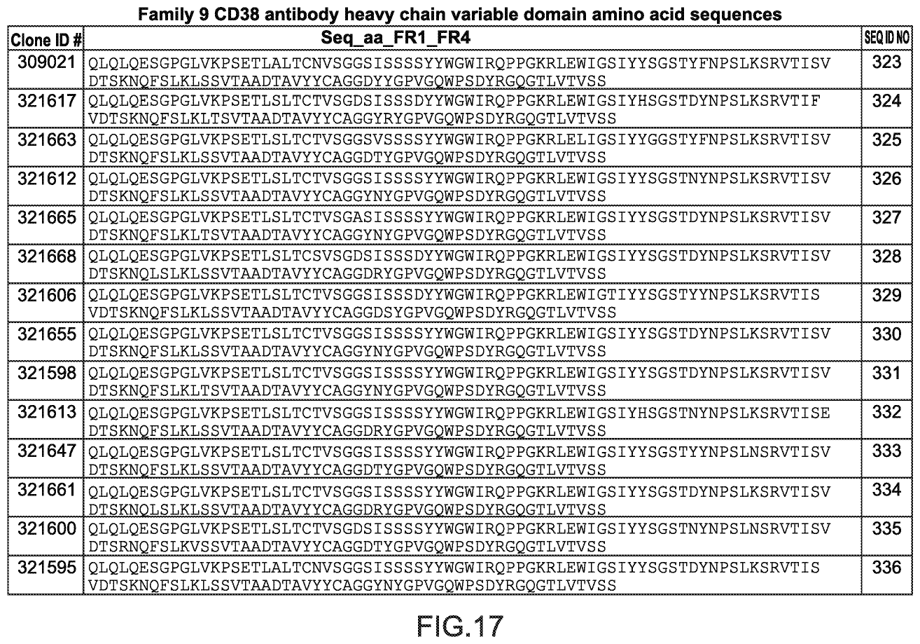

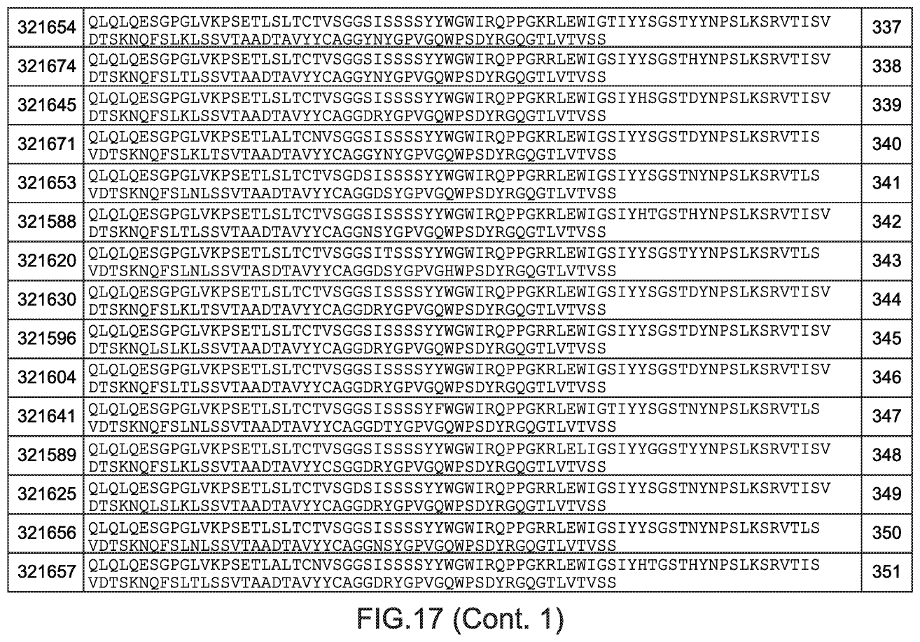

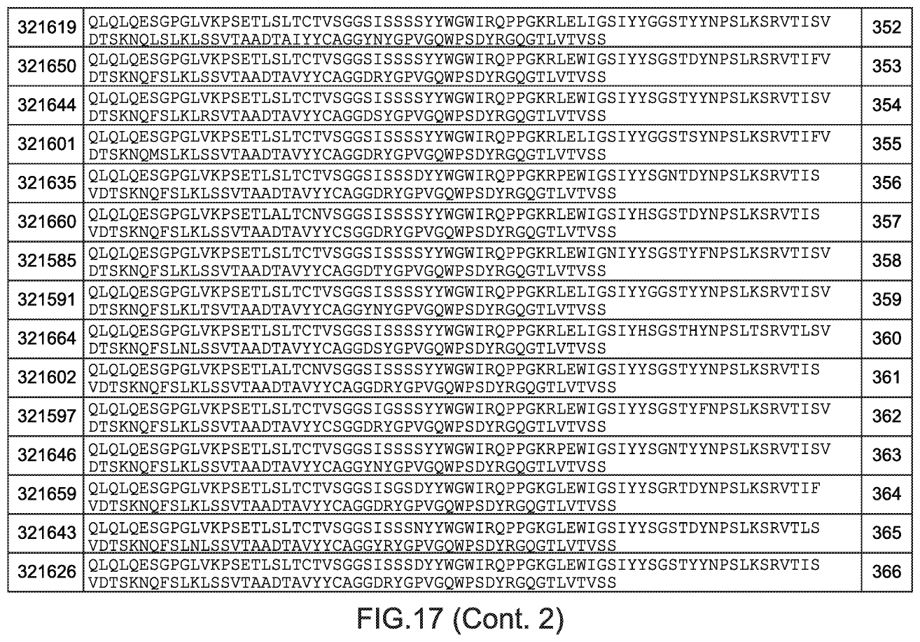

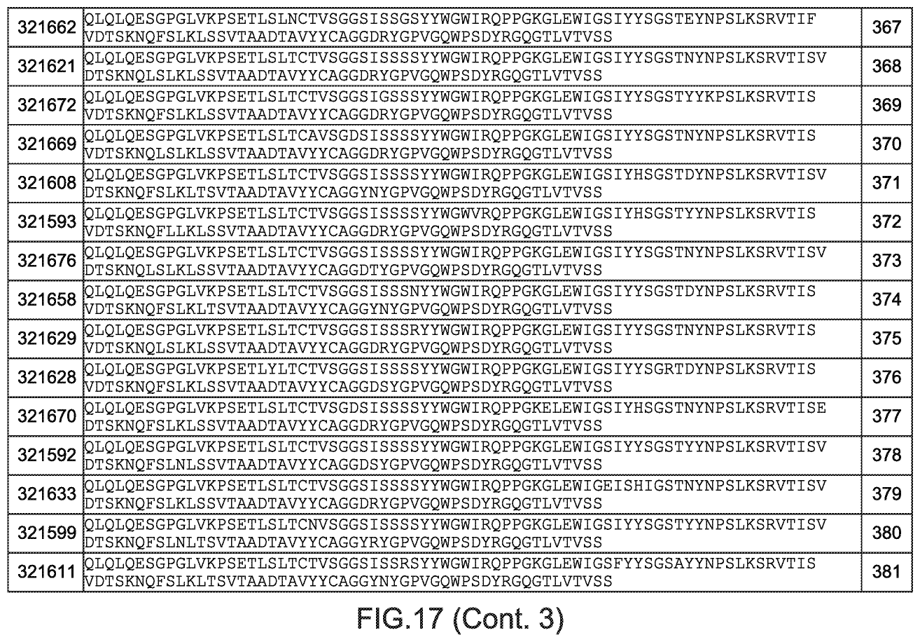

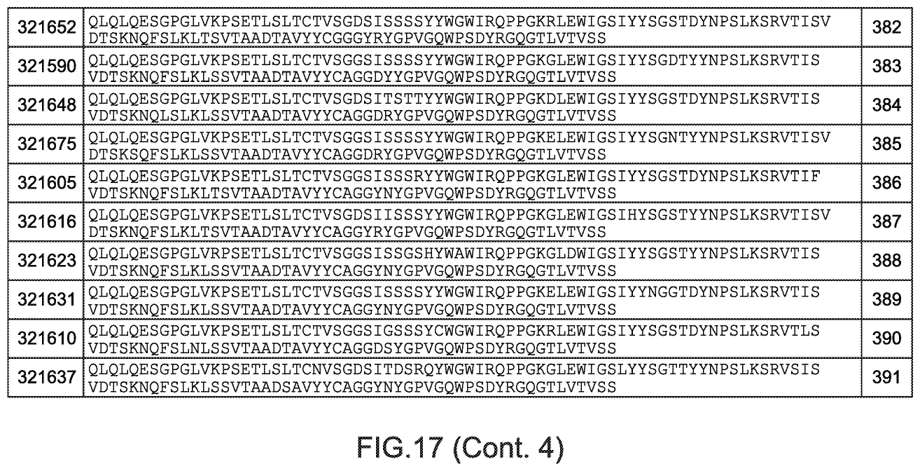

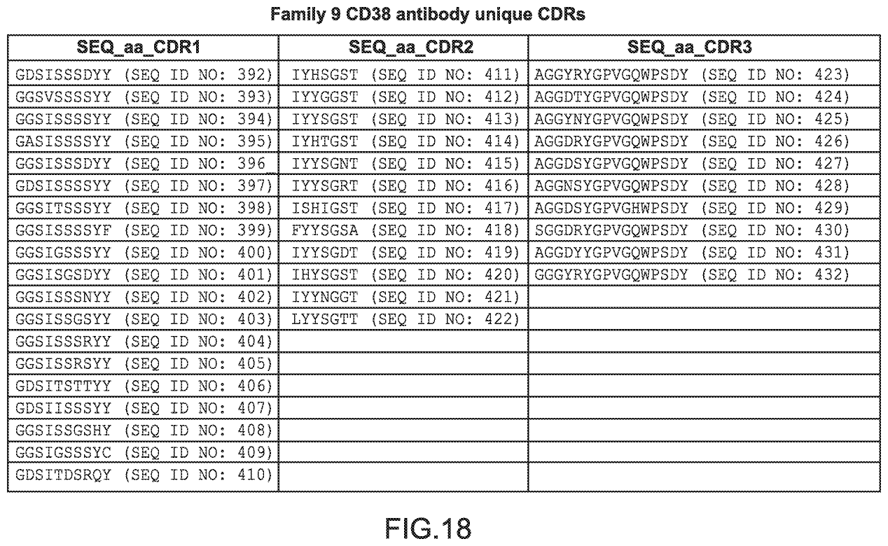

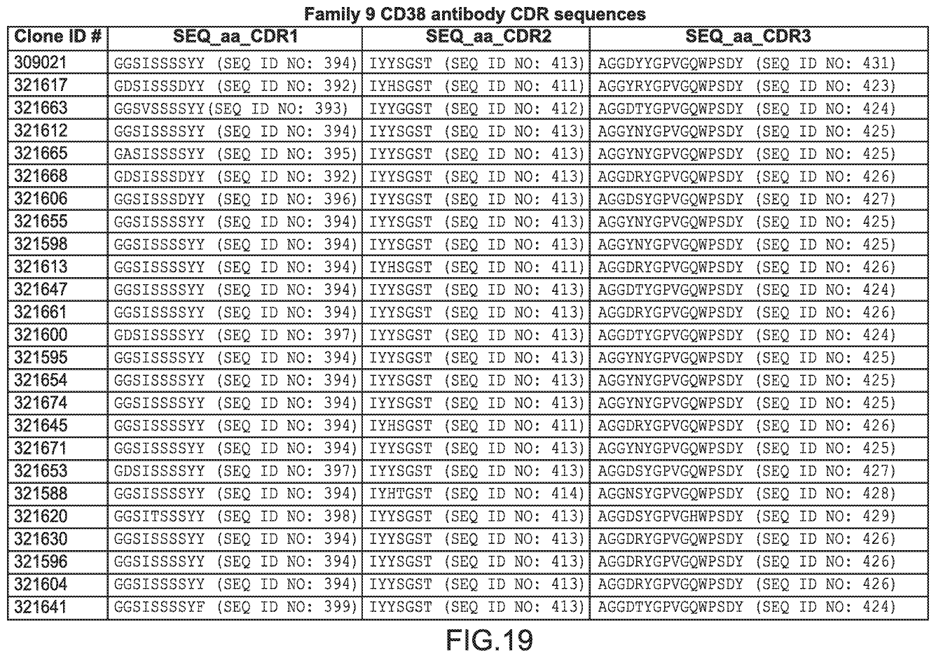

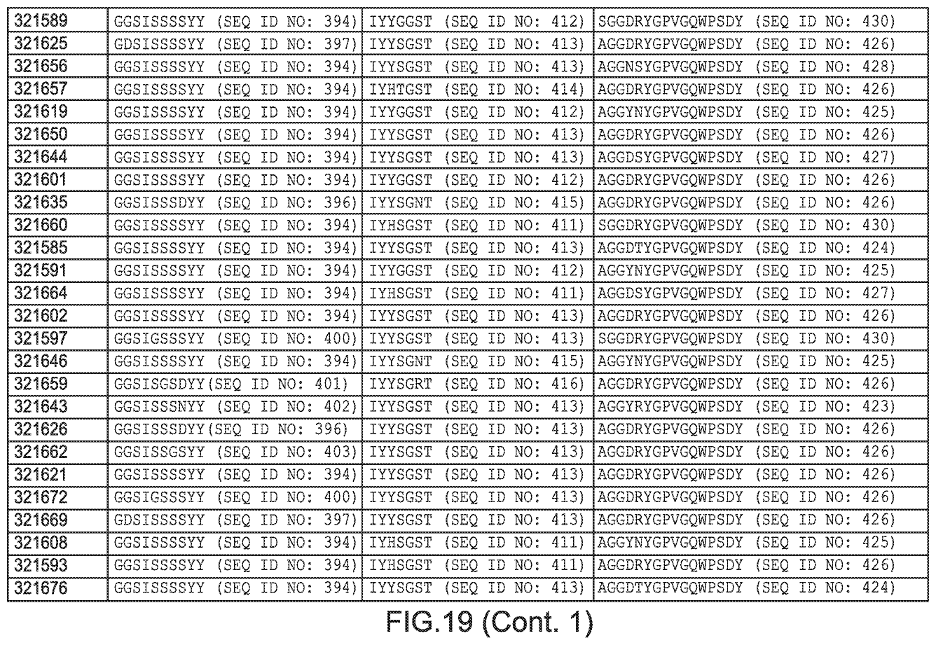

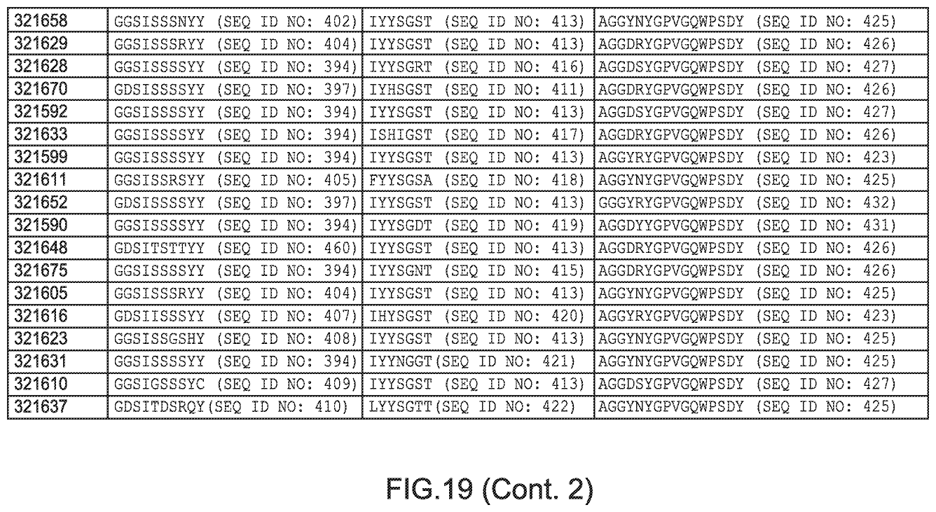

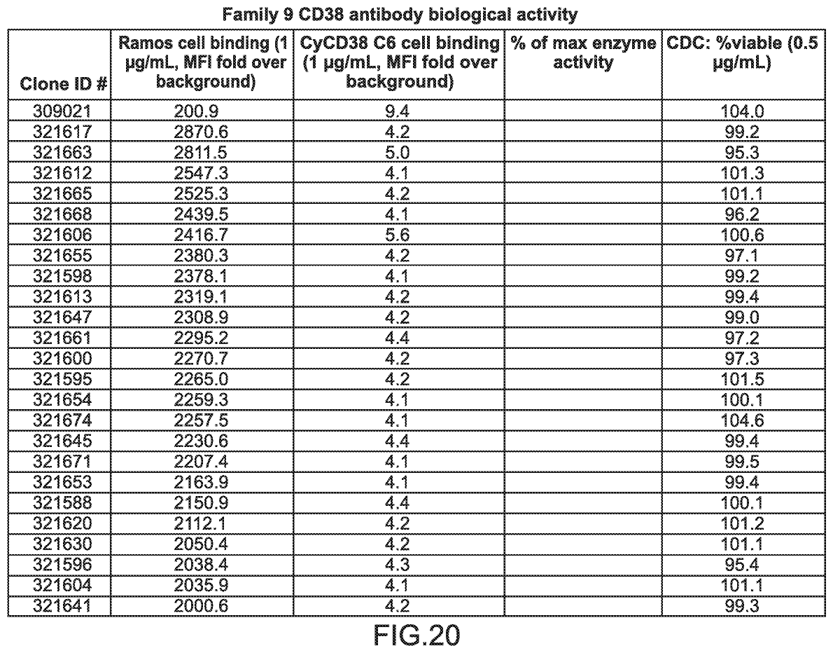

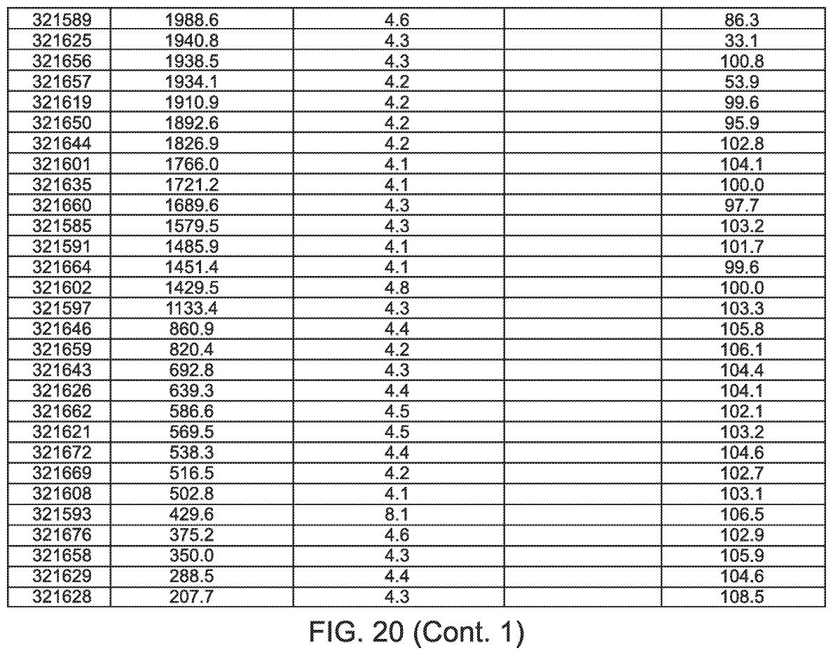

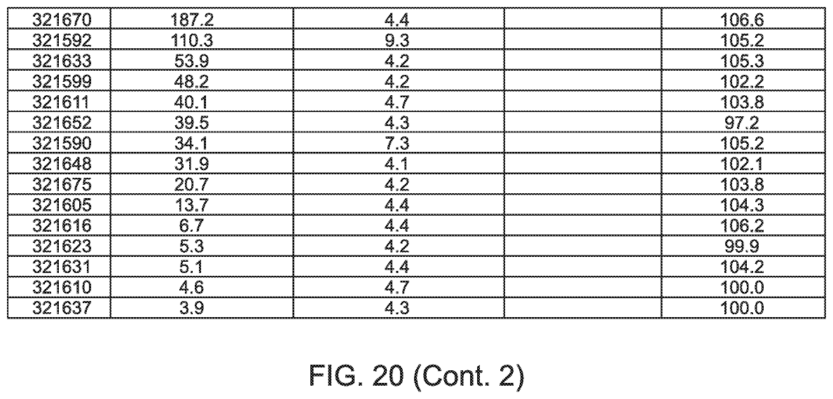

As part of the present invention, human IgG heavy chain anti-CDR3 antibody families with unique CDR3 sequences from UniRat™ animals (UniAb™) were identified that bind human CD38 in ELISA (recombinant CD38 extracellular domain) protein and cell-binding assays. Heavy chain variable region (VH) sequences comprising five sequence families (F01, F03, F04, F07, and F09) (see FIGS. 1-20) are positive for human CD38 protein binding and/or for binding to CD38+ cells, and are all are negative for binding to cells that do not express CD38. UniAbs™ from these five sequence families fall into two broad competition groups based on the ability of Daratumamab and Isatuximab to block UniAb™ binding to CD38+ cells. Combinations of two or more UniAbs™ binding distinct epitopes induce potent CDC and direct apoptosis, where the same UniAbs™ by themselves do not induce either of these effector functions. Combinations of UniAbs™ also inhibited enzymatic activities more potently than the individual UniAbs™.

Members of the antibody families herein bind CD38-positive Burkitt's lymphoma cell line Ramos, and some are cross-reactive with the CD38 protein of Cynomolgus macaque. In addition, they can be engineered to provide cross-reactivity with the CD38 protein of any animal species, if desired.

The anti-ectoenzyme heavy chain antibodies, including anti-CD38 heavy chain antibodies, such as UniAbs™ herein may have an affinity for CD38 with a Kd of from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating, e.g. blocking, a CD38 biological activity, including in vitro assays, pre-clinical models, and clinical trials, as well as assessment of potential toxicity.

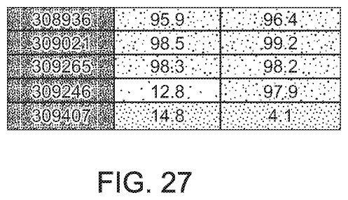

Heavy chain antibodies binding to non-overlapping epitopes on an ectoenzyme target, including but not limited to anti-CD38 heavy chain antibodies, e.g. UniAbs™ can be identified by competition binding assays, such as enzyme-linked immunoassays (ELISA assays) or flow cytometric competitive binding assays. For example, one can use competition between known antibodies binding to the target antigen and the antibody of interest. By using this approach, one can divide a set of antibodies into those that compete with the reference antibody and those that do not. The non-competing antibodies are identified as binding to a distinct epitope that does not overlap with the epitope bound by the reference antibody. Often, one antibody is immobilized, the antigen is bound, and a second, labeled (e.g. biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc). For further details see the Examples and FIG. 27.

Typically, an antibody “competes” with a reference antibody if it causes about 15-100% reduction in the binding of the reference antibody to the target antigen, as determined by standard techniques, such as by the competition binding assays described above. In various embodiments, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

In one embodiment, a bispecific, bivalent heavy chain antibody having binding affinity to a first CD38 epitope and a second, non-overlapping CD38 epitope comprises a first polypeptide having binding affinity to the first CD38 epitope comprising an antigen-binding domain of a heavy-chain antibody comprising a CDR1 sequence of SEQ ID NO: 150, a CDR2 sequence of SEQ ID NO: 92, and a CDR3 sequence of SEQ ID NO: 168, at least a portion of a hinge region, and a CH domain comprising a CH2 domain and a CH3 domain, and a second polypeptide having binding affinity to the second CD38 epitope comprising an antigen-binding domain of a heavy-chain antibody comprising a CDR1 sequence of SEQ ID NO: 393, a CDR2 sequence of SEQ ID NO: 412, and a CDR3 sequence of SEQ ID NO: 424, at least a portion of a hinge region, and a CH domain comprising a CH2 domain and a CH3 domain, and an asymmetric interface between the CH2 domain of the first polypeptide and the CH2 domain of the second polypeptide. In certain preferred embodiments, this bispecific, bivalent heavy chain antibody comprises an Fc region that is a human IgG1 Fc region, a human IgG4 Fc region, a silenced human IgG1 Fc region, or a silenced human IgG4 Fc region.

In one embodiment, a bispecific, tetravalent heavy chain antibody having binding affinity to a first CD38 epitope and a second, non-overlapping CD38 epitope includes two identical polypeptides, each polypeptide comprising a first antigen-binding domain of a heavy-chain antibody having binding affinity to the first CD38 epitope, comprising a CDR1 sequence of SEQ ID NO: 150, a CDR2 sequence of SEQ ID NO: 92, and a CDR3 sequence of SEQ ID NO: 168, a second antigen-binding domain of a heavy-chain antibody having binding affinity to the second CD38 epitope, comprising a CDR1 sequence of SEQ ID NO: 393, a CDR2 sequence of SEQ ID NO: 412, and a CDR3 sequence of SEQ ID NO: 424, at least a portion of a hinge region, and a CH domain comprising a CH2 domain and a CH3 domain. In certain embodiments, this heavy chain antibody comprises an Fc region that is a human IgG1 Fc region, a human IgG4 Fc region, a silenced human IgG1 Fc region, or a silenced human IgG4 Fc region.

In another embodiment, a bispecific, tetravalent heavy chain antibody having binding affinity to a first CD38 epitope and a second, non-overlapping CD38 epitope comprises a first and a second heavy chain polypeptide, wherein the first heavy chain polypeptide comprises two antigen-binding domains of a heavy-chain antibody having binding affinity to the first CD38 epitope, each antigen-binding domain comprising a CDR1 sequence of SEQ ID NO: 150, a CDR2 sequence of SEQ ID NO: 92, and a CDR3 sequence of SEQ ID NO: 168, at least a portion of a hinge region, and a CH domain comprising a CH2 domain and a CH3 domain, and an asymmetric interface between the CH2 domain of the first polypeptide and the CH2 domain of the second polypeptide, and wherein the second heavy chain polypeptide comprises two antigen-binding domains of a heavy-chain antibody having binding affinity to the second CD38 epitope, each antigen-binding domain comprising a CDR1 sequence of SEQ ID NO: 393, a CDR2 sequence of SEQ ID NO: 412, and a CDR3 sequence of SEQ ID NO: 424, at least a portion of a hinge region, and a CH domain comprising a CH2 domain and a CH3 domain, and an asymmetric interface between the CH2 domain of the first polypeptide and the CH2 domain of the second polypeptide. In certain preferred embodiments, this heavy chain antibody comprises an Fc region that is a human IgG1 Fc region, a human IgG4 Fc region, a silenced human IgG1 Fc region, or a silenced human IgG4 Fc region.

In some embodiments, two or more of the antigen-binding domains described herein are combined into a single molecule, e.g., a bispecific, tetravalent antibody, in accordance with methods described herein and/or known in the art. In one embodiment, a bispecific, tetravalent antibody of the invention includes heavy chain variable region sequences of clone ID 321986 and clone ID 321663. Antibodies in accordance with embodiments of the invention can have any suitable orientation of heavy chain variable region sequences (N terminus to C terminus, or C terminus to N terminus) along each polypeptide subunit of the binding compound. In certain embodiments, the orientation of the heavy chain variable region sequences along each polypeptide subunit, from N terminus to C terminus, is: VH 321663, VH 321986. In certain embodiments, the orientation of the heavy chain variable region sequences along each polypeptide subunit, from N terminus to C terminus, is: VH 321986, VH 321663. Tetravalent antibodies in accordance with some embodiments of the invention include linker sequences that are positioned in a suitable location. In some embodiments, a linker is positioned between the first and second VH domains on each polypeptide subunit. In some embodiments, a linker is placed proximally or distally to a give VH domain, e.g., a linker is positioned on a C-terminal end of a VH domain and/or an N terminal end of a VH domain.

### Pharmaceutical Compositions, Uses and Methods of Treatment

It is another aspect of the present invention to provide pharmaceutical compositions comprising one or more antibodies of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one embodiment, the pharmaceutical composition comprises two or more heavy chain only antibodies binding to non-overlapping epitopes on an ectoenzyme, such as, for example, CD38, CD73, or CD39. In a preferred embodiment, the pharmaceutical compositions comprise synergistic combinations of two or more heavy chain only antibodies binding to non-ovelapping epitopes of an ectoenzyme, such a, for example, CD38, CD73, or CD39.

In another embodiment, the pharmaceutical composition comprises a multi-specific (including bispecific) heavy chain only antibody with binding specificity for two or more non-overlapping epitopes on an ectoenzyme, such as, for example, CD38, CD73, or CD39. In a preferred embodiment, the pharmaceutical composition comprises a multi-specific (including bispecific) heavy chain only antibody with binding specificity to two or more non-overlapping epitopes on an ectoenzyme, e.g. CD38, CD73, or CD39, having improved properties relative to any of the monospecific antibodies binding to the same epitopes.

Pharmaceutical composition of the antibodies used in accordance with the present invention are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The antibodies herein can be administered by intravenous injection or infusion or subcutaneously. For injection administration, the antibodies herein can be formulated in aqueous solutions, preferably in physiologically-compatible buffers to reduce discomfort at the site of injection. The solution can contain carriers, excipients, or stabilizers as discussed above. Alternatively antibodies can be in lyophilized form for constitution with a suitable vehicle, e.g., sterile pyrogen-free water, before use.

Anti-CD38 antibody formulations are disclosed, for example, in U.S. Pat. No. 9,034,324. Similar formulations can be used for the heavy chain antibodies, including UniAbs™, of the present invention. Subcutaneous antibody formulations are described, for example, in US 20160355591 and US 20160166689.

### Methods of Use

The heavy chain only antibodies binding to non-overlapping epitopes on an ectoenzyme, combinations, including synergistic combinations, of such antibodies, multi-specific antibodies with binding specificities to two or more non-overlapping epitopes on an ectoenzyme, and pharmaceutical compositions comprising such antibodies and antibody combinations, can be used to target diseases and conditions characterized by the expression of the target ectoenzyme.

In various embodiments, the ectoenzyme is selected from the group consisting of CD10, CD13, CD26, CD38, CD39, CD73, CD156b, CD156c, CD157, CD203, VAP1, ART2, and MT1-MMP.

In a particular embodiment, the ectoenzyme is CD38, CD73 and/or CD39.

CD38 is a 46-kDa type II transmembrane glycoprotein with a short 20-aa N-terminal cytoplasmic tail and a long 256-aa extracellular domain (Malavasi et al., Immunol. Today, 1994, 15:95-97). Due to its high level of expression in a number of hematological malignancies, including multiple myeloma (MM), non-Hodgkin's lymphoma (reviewed in Shallis et al., Cancer Immunol. Immunother., 2017, 66(6):697-703), B-cell chronic lymphocylic leukemia (CLL) (Vaisitti et al., Leukemia, 2015, 29″356-368), B-cell acute lymphoblastic leukemia (ALL), an dT-cell ALL, CD38 is a promising target for antibody-based therapeutics to treat hematological malignancies. CD38 has also be implicated as a key actor in age-related micorinamide adenine dinucleotide (NAD) decline, and it has been suggested that CD38 inhibition, combined with NAD precursors may serve as a potential therapy for metabolic dysfunction and age-related diseases (see, e.g. Camacho-Pereira et al., Cell Metabolism 2016, 23:1127-1139). CD38 has also been described as being involved in the development of airway hyper-responsiveness, a hallmark feature of asthma, and has been suggested as a target to treat such conditions.

The heavy chain only anti-CD38 antibodies, antibody combinations, multi-specific antibodies, and pharmaceutical compositions herein can be used to target diseases and conditions characterized by the expression or overexpression of CD38, including, without limitation, the conditions and diseases listed above.

In one aspect, the CD38 heavy chain antibodies and pharmaceutical compositions herein can be used to treat hematological malignancies characterized by the expression of CD38, including multiple myeloma (MM), non-Hodgkin's lymphoma, B-cell chronic lymphocylic leukemia (CLL), B-cell acute lymphoblastic leukemia (ALL), an dT-cell ALL. The CD38 heavy chain antibodies and pharmaceutical compositions of the present invention can also be used to treat asthma and other conditions characterized by airway hyper-responsiveness, and age-related, and metabolic dysfunction characterized by micorinamide adenine dinucleotide (NAD) decline.

MM is a B-cell malignancy characterized by a monoclonal expansion and accumulation of abnormal plasma cells in the bone marrow compartment. Current therapies for MM often cause remissions, but nearly all patients eventually relapse and die. There is substantial evidence of an immune-mediated elimination of myeloma cells in the setting of allogeneic hematopoietic stem cell transplantation; however, the toxicity of this approach is high, and few patients are cured. Although some monoclonal antibodies have shown promise for treating MM in preclinical studies and early clinical trials, consistent clinical efficacy of any monoclonal antibody therapy for MM has not been conclusively demonstrated. There is therefore a great need for new therapies, including immunotherapies for MM (see, e.g. Shallis et al, supra).

CD73 has been described to function as an ectoenzyme to produce extracellular adenosine, which promotes tumor growth by limiting antitumor T-cell immunity via adenosine receptor signaling. CD73 is expressed in certain cancers, such as breast, colon and prostate cancers. Results with small molecule inhibitors or monoclonal antibodies targeting CD73 in murine tumor models, suggest the potential of targeted CD73 therapy, including immunotherapy, to control growth of tumors characterized by the expression of CD73, as monotherapy or in combination with other anticancer agents, such as anti-PD1 and/or anti-CTLA-4 antibodies. See, e.g. B Zhang, Cancer Res; 2010, 70(16), 6407-11; Allard et al., Clinical Cancer Res, 2013, 19(20):5626-5635.

CD39 and CD73 have been widely considered pivotal in the generation of immunosuppressive microenvironments through adenosine production. Upregulation of CD39 has been reported in a number of epithelial and hematological malignancies and its expression in chronic lymphocytic leukemia has been shown to correlate with poor prognosis (Pulte et al., 2011, Clin Lymphoma Myeloma Leuk. 2011;11:367-372; Bastid et al., 2013, Oncogene, 32:1743-1751; Bastid et al., 2015, Cancer Immunol Res., 3:254-265. CD39 is also highly expressed on regulatory T-cells (Tregs) and is required for their suppressive function as demonstrated with impaired suppressive activity of Tregs in CD39-null mice (Deaglio et al., 2007, J Exp Med., 204:1257-1265). It has been suggested that CD39 may help drive tumorigenesis by its enhanced enzymatic activity either on Tregs, tumor-associated stroma or on malignant epithelial cells, resulting in adenosine-mediated immunosuppression of anti-tumor T- and natural killer (NK) cells as well as neutralization of ATP-induced cell death by chemotherapy (Bastid et al., 2013 and 2015, supra; Feng et al., 2011, Neoplasia, 13:206-216). Modulation of the immunosuppressive CD39/CD73-adenosine pathway has been suggested as a promising immunotherapeutic strategy for cancer therapy (Sitkovsky et al., 2014, Cancer Immunol Res. 2:598-605). See also, Hayes et al., Am J Trans Res, 2015, 7(6):1181-1188.

For review of the role of CD73 and CD39 ectonucleotidases in T cell differentiation see, e.g. Bono et al., FEBS Letters, 2015, 589:3454-3460.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage the agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Typically, compositions are prepared as injectables, either as liquid solutions or suspensions;

solid forms suitable for solution in, or suspension in, liquid vehicles prior to injection can also be prepared. The pharmaceutical compositions herein are suitable for intravenous or subcutaneous administration, directly or after reconstitution of solid (e.g. lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other ablative agent dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent, e.g. a chemotherapeutic drug, etc. Kits typically include a label indicating the intended use of the contents of the kit. The term label includes any writing, or recorded material supplied on or with the kit, or which otherwise accompanies the kit.

The invention now being fully described, it will be apparent to one of ordinary skill in the art that various changes and modifications can be made without departing from the spirit or scope of the invention.

### Materials and Methods

### CD38 Protein Binding

The kinetic experiments to determine the antigen-antibody affinities were performed on the Octet QK-384 system (ForteBio). Anti-human IgG Fc Capture (AHC) biosensors (Forte Bio, Part No: 18-5064) were hydrated in assay buffer (1×PBS, 0.1% BSA, 0.02% Tween-20, pH 7.2) and preconditioned in 100 mM Glycine pH 1.5. A baseline was established in the assay buffer for 120 seconds. AHC biosensors were then immobilized with UniAbs™ at a concentration of 5 μg/mL for 120 seconds. Another baseline (120 seconds) was established in the assay buffer. Next, they were then dipped into a 7-point, 1:2 dilution series of the antigen cyCD38 (Sino Biologics—90050-CO8H) in the assay buffer, starting from 250 nM. The last well of the analyte column contained only assay buffer to test for non-specific binding between the buffer and the loaded biosensors, and was used as a reference well. Association was observed for 600 seconds, followed by dissociation for 900 seconds. Data analysis was performed using Octet Data Analysis v9.0 (ForteBio). Binding kinetics were analyzed using a standard 1:1 binding model.

### CD38 Cell Binding

Binding to CD38 positive cells was assessed by flow cytometry (Guava easyCyte 8HT, EMD Millipore) using the Ramos cell line (ATCC). Briefly, 100,000 target cells were stained with a dilution series of purified UniAbs™ for 30 minutes at 4° C. Following incubation, the cells were washed twice with flow cytometry buffer (1×PBS, 1% BSA, 0.1% NaN₃) and stained with goat F(ab′)₂ anti-human IgG conjugated to R-phycoerythrin (PE) (Southern Biotech, cat. #2042-09) to detect cell-bound antibodies. After a 20-minute incubation at 4° C., the cells were washed twice with flow cytometry buffer and then mean fluorescence intensity (MFI) was measured by flow cytometry. EC₅₀ values were calculated using GraphPad Prism 7. Binding to cynomolgus CD38 positive cells was determined using the same protocol with the following modifications: the target cells were from a rat C6 cell line (ATCC) stably transfected to express the extracellular domain of cynomolgus CD38 and each antibody was tested at a single concentration (˜1.7 μg/mL) so EC50 values were not calculated.

### Complement Dependent Cytotoxicity (CDC)

Complement dependent cytotoxicity (CDC) was determined for each anti-CD38 UniAb™ using the CD38 positive Daudi or Ramos cell lines (ATCC). In summary, 20,000 target cells were opsonized with either a single concentration of 1 μg/mL of purified UniAb™, or a dose range of purified UniAb for 10 minutes at room temperature. Following incubation, either human complement serum (Innovative Research, cat. #IPLA-CSER) was added to a final concentration of 17% or rabbit complement serum (Sigma-Aldrich, cat. #S7764) was added to a final concentration of 5% and incubated (37° C., 8% CO₂) for 3.5 hours or 30 minutes respectively. After incubation, cell viability was measured by indirect quantification of ATP through addition of an ATP-dependent luminescence reagent, Cell Titer Glo 2.0 (Promega, cat. #G9232). Luminescence signal was recorded using a Spectramax i3x plate reader (Molecular Devices) and percent viability was determined by comparison to cells treated with an isotype control antibody.

### Antibody Dependent Cellular Cytotoxicity (ADCC)

Antibody dependent cellular cytotoxicity (ADCC) was assessed using a cell-based ADCC Reporter Bioassay (Promega, cat. #G7010). In brief, 12,500 CD38 positive Ramos target cells (ATCC) were added to the wells of a 96-well plate and treated with a dilution series of each anti-CD38 UniAb™. Next, reporter cells expressing FcγRIIIa as well as a luciferase reporter under control of a NFAT response element were added at an E:T ratio of 6:1 and incubated for 6 hours in a tissue culture incubator (37° C., 8% CO₂). After the addition of Bio-Glo luciferase assay substrate, luminescence was measured using a Spectramax i3x plate reader (Molecular Devices). Increasing luminescent reporter signal indicates more ADCC activity. EC50 values were calculated using GraphPad Prism software (sigmoidal, 4PL curve fit).

### Antibody-Induced Direct Apoptosis

Cytotoxicity through antibody-induced direct apoptosis was analyzed using CD38 positive Ramos cells (ATCC). In summary, 45,000 target cells were treated with either 2 μg/mL of purified UniAbs™ or a dose range of purified UniAbs™ for 48 hours (37° C., 8% CO₂). Following incubation, the cells were washed twice with Annexin-V binding buffer (BioLegend, cat. #422201) and stained with Annexin V and 7-AAD (BioLegend, cat. #640945 and 420404). The samples were then analyzed by flow cytometry (Guava easyCyte 8HT, EMD Millipore) and the percentage of viable cells was determined as the population negative for Annexin V and 7AAD.

### Antibody-Induced Indirect Apoptosis

To measure apoptosis mediated through Fc cross-linking, CD38 positive Ramos target cells (ATCC) were treated with 0.4 μg/mL of anti-CD38 UniAbs™ and 1.6 μg/mL of purified goat F(ab′)2 anti-human IgG Fc (Abcam, cat. #ab98526). After a 24-hour incubation (37° C., 8% CO₂) the cells were washed and resuspended in Annexin V binding buffer (BioLegend, cat. #422201) and stained with Annexin V and 7-AAD (BioLegend, cat. #640945 and 420404). The samples were then analyzed by flow cytometry (Guava easyCyte 8HT, EMD Millipore) and the percentage of viable cells was determined as the population negative for Annexin V and 7AAD.

### CD38 Enzymatic Activity

To measure inhibition of CD38 cyclase activity, recombinant human CD38 (Sino Biological, 10818-H08H) was incubated with 50 μg/mL of each purified anti-CD38 UniAb™ in cyclase activity buffer (50 mM MES pH 6.5) for 15 minutes at room temperature. After incubation, nicotinamide guanine dinucleotide (Sigma Aldrich, cat. #N5131) was added to a final concentration of 150 μM. Production of the fluorescent molecule cyclic GDP ribose was measured at 1 hour (ex 300 nm/em 410 nm) using a Spectramax i3x plate reader (Molecular Devices). Cyclase enzyme inhibition was assessed by comparing signal from UniAb™-treated wells to the percent of total enzymatic activity observed when CD38 protein was treated with an isotype control antibody (max).

### Example 1: Genetically Engineered Rats Expressing Heavy Chain-Only Antibodies

A ‘human- rat’ IgH locus was constructed and assembled in several parts. This involved the modification and joining of rat C region genes downstream of human JHs and subsequently, the upstream addition of the human V_{H}6-D-segment region. Two BACs with separate clusters of human V_{H} genes [BAC6 and BAC3] were then co-injected with the BAC termed Georg, encoding the assembled and modified region comprising human V_{H}6, all Ds, all J_{H}s, and modified rat Cγ2a/1/2b (ΔC_{H}1).

Transgenic rats carrying artificial heavy chain immunoglobulin loci in unrearranged configuration were generated. The IgG2a(ΔC_{H}1), IgG1(ΔC_{H}1), IgG2b(ΔC_{H}1) genes lacked the C_{H}1 segment. The constant region genes IgE, IgA and 3′ enhancer were included in Georg BAC. RT-PCR and serum analysis (ELISA) of transgenic rats revealed productive rearrangement of transgenic immunoglobulin loci and expression of heavy chain only antibodies of various isotypes in serum. Transgenic rats were cross-bred with rats with mutated endogenous heavy chain and light chain loci previously described in US Patent Publication No. 2009/0098134 A1. Analysis of such animals demonstrated inactivation of rat immunoglobulin heavy and light chain expression and high level expression of heavy chain antibodies with variable regions encoded by human V, D, and J genes. Immunization of transgenic rats resulted in production of high titer serum responses of antigen-specific heavy chain antibodies. These transgenic rats expressing heavy chain antibodies with a human VDJ region were called UniRats™.

### Example 2: Immunization of UniRats™ and Determination of Serum Titers

### Immunization with Recombinant Extracellular Domain of BCMA

Twelve UniRat™ animals (6 HC27, 6 HC28) were immunized with recombinant human CD38 protein. The animals were immunized according to standard protocol using a Titermax/Alhydrogel adjuvant. Recombinant extracellular domain of CD38 was purchased from R&D Systems and was diluted with sterile saline and combined with adjuvant. The immunogen was combined with Titermax and Alhydrogel adjuvants. The first immunization (priming) with immunogen in Titermax was administered in the left and right legs. Subsequent boosting immunizations were done in the presence of Alhydrogel and three days before harvest boosts were performed with immunogens in PBS. Serum was collected from rats at the final bleed to determine serum titers.

### Serum Titer Results

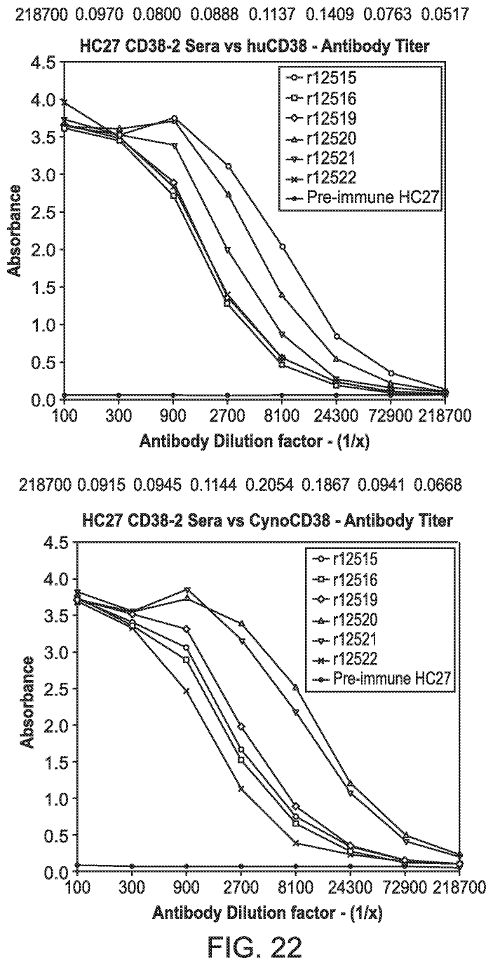

Binding activity for serum titer dilutions were tested against the immunogen as shown in FIG. 22 for six animals. Serum taken from all animals showed reactivity to the recombinant protein and did not bind control antigens.

### Example 3: Gene Assembly, Expression and Sequencing

cDNAs encoding heavy chain only antibodies highly expressed in lymph node cells were selected for gene assembly and cloned into an expression vector. Subsequently, these heavy chain sequences were expressed in HEK cells as UniAb™ heavy chain only antibodies (CH1 deleted, no light chain).

FIGS. 1, 5, 9, 13, and 17 show the heavy chain variable domain amino acid sequences of anti-CD38 UniAb™ families 1, 3, 4, 7, and 9, respectively.

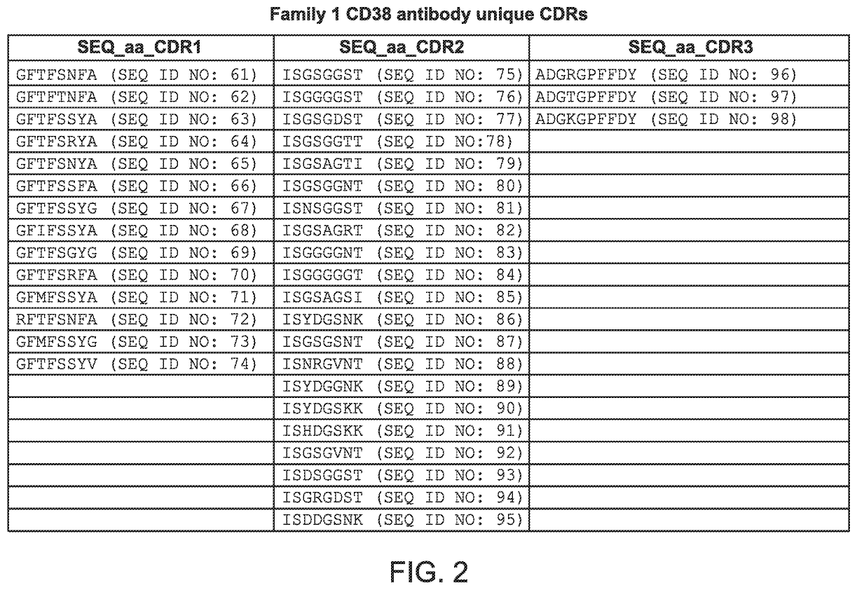

FIGS. 2, 6, 10, 14, and 18 show unique CDR1-3 sequences of anti-CD38 UniAb™ families 1, 3, 4, 7, and 9, respectively.

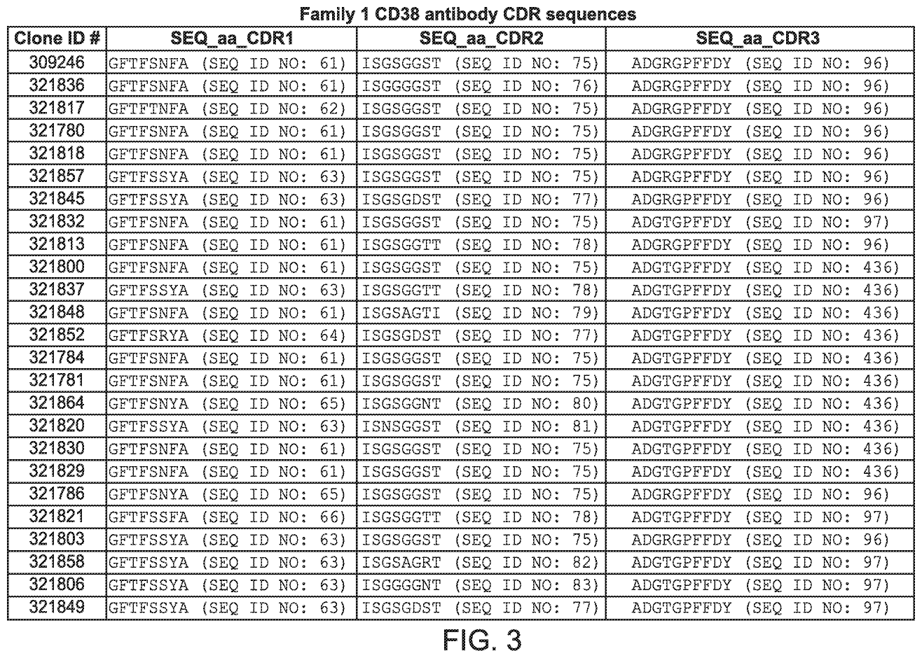

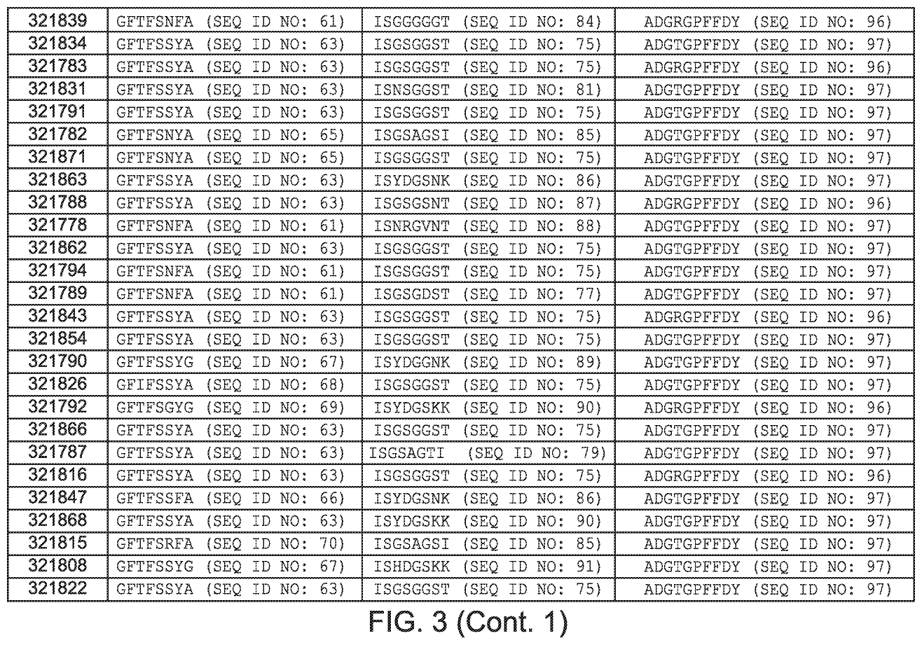

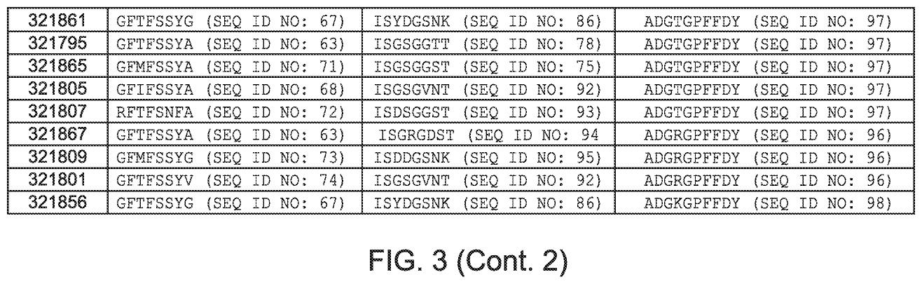

FIGS. 3, 7, 11, 15, and 19 show the CDR1-CDR3 sequences of the listed anti-CD38 UniAb™ antibodies of families 1, 3, 4, 7, and 9, respectively.

### Example 4: Cell Binding, Enzymatic and CDC Activities

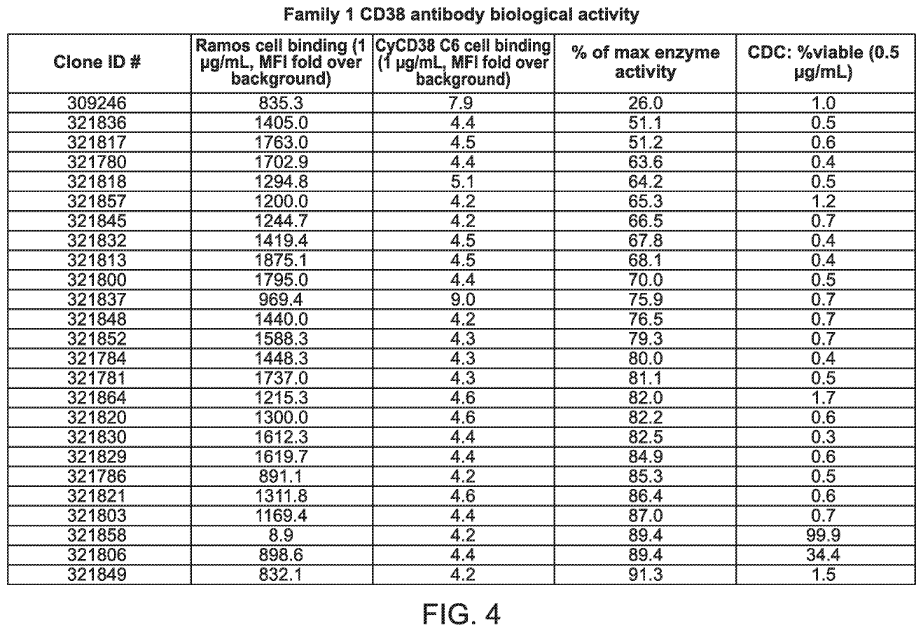

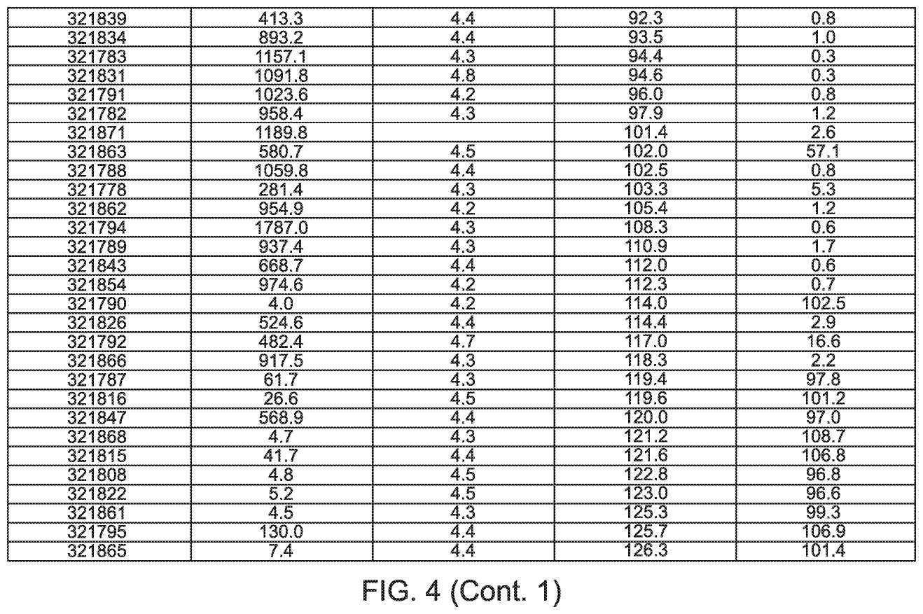

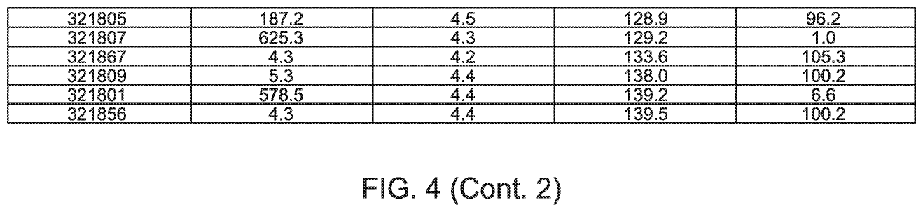

FIGS. 4, 8, 12, 16, and 20 show the Ramos cell binding, CyCD38 C6 cell binding, enzymatic activities and CDC activities of the listed anti-CD38 UniAb™ antibodies of families 1, 3, 4, 7, and 9, respectively. The first column indicates the clone ID of the UniAb™ tested. The second column indicates the mean fluorescent intensity (MFI) of cell binding to Ramos cells divided by the background MFI of a control antibody incubated with Ramos. The third column indicates the mean fluorescent intensity (MFI) of cell binding to rat C6 cells transfected with cynomolgus CD38 divided by the background MFI of a control antibody incubated with the same cells. The fourth column indicates percentage enzymatic activity of recombinant CD38 in the presence of the respective CD38-binding UniAbs™ versus control UniAb™.

### Example 5: Further Characterization of Anti-CD38 UniAbs™

As shown in FIG.23, UniAbs™ representing five unique heavy chain CDR3 sequence families exhibit a variety of functional behaviors with each family displaying a unique set of characteristics. A single lead VH sequence was selected from each of the five CDR3 sequence families for additional functional screening in IgG1 UniAb™ format. In some assays, Daratumumab and Isatuximab were included as reference controls. Each UniAb was characterized for its binding to human and cyno CD38 proteins and binding to cells expressing either human or cyno CD38. In addition, the UniAbs™ were assessed for ability to inhibit the natural cyclase (enzyme) activity of CD38 as well as the ability to stimulate indirect apoptosis, direct apoptosis, ADCC and CDC on CD38-expressing mammalian cells under the appropriate assay conditions.

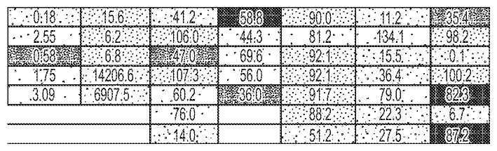

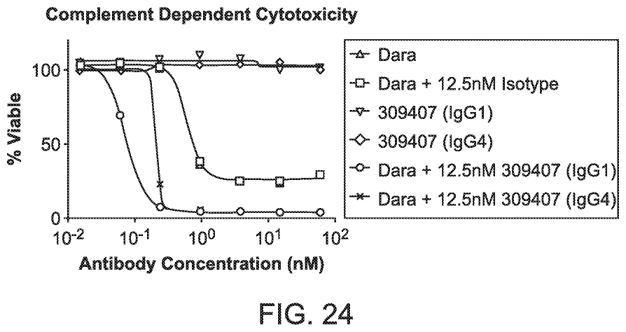

FIG. 24 shows CDC of different combinations UniAb™ 309407 (at 12.5 nM) mixed with Daratumumab at different concentrations. UniAb™ 309407 did not lyse Ramos cells by CDC by itself. Daratumumab mixed with UniAb 309407 was more potent than Daratumumab alone. UniAb 309407 on a human IgG4 background also augmented CDC activity of Daratumumab. IgG4 does not bind complement. This indicates that binding of UniAb 309407 to CD38 modulates CDC activity of an antibody binding a non-overlapping epitope.

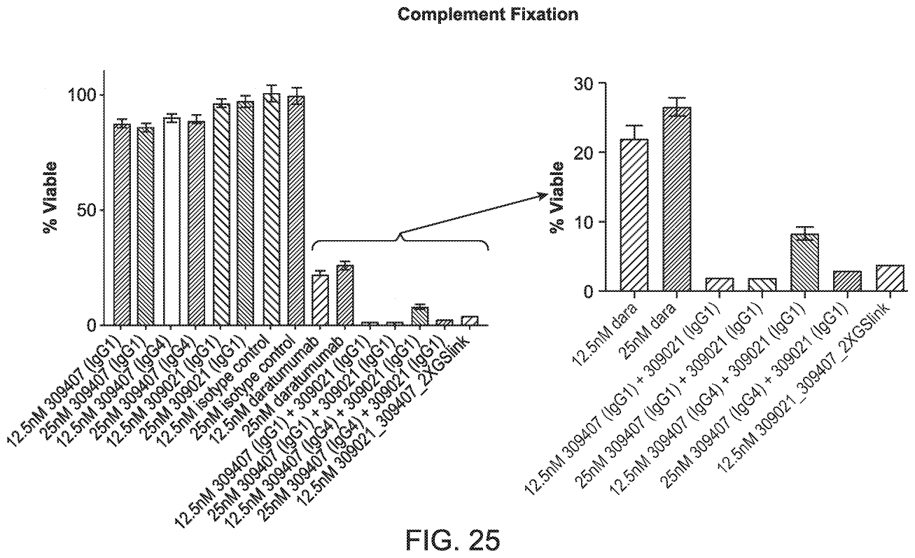

FIG. 25 shows complement fixation of combinations of UniAbs™ and a tetravalent bispecific UniAb comprising VH domains of ID309021 and ID309407. These two UniAbs™ and their VH domains bind 2 non-overlapping epitopes on CD38. Combining these two CD38 binders in a single tetravalent antibody (309021_309407_2XGSlink) yielded strong complement fixation and killing of tumor cells. Mixtures of UniAbs™ and tetravalent bispecific UniAb induced more efficacious CDC of Ramos cells compared to Daratumumab. Individual UniAbs™ did not induce CDC.

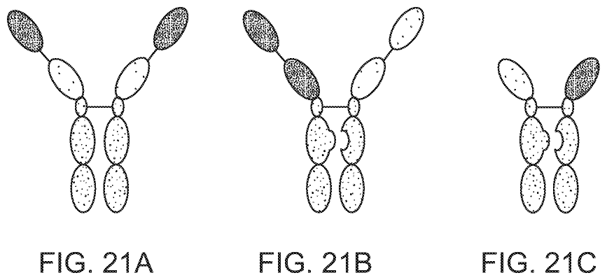

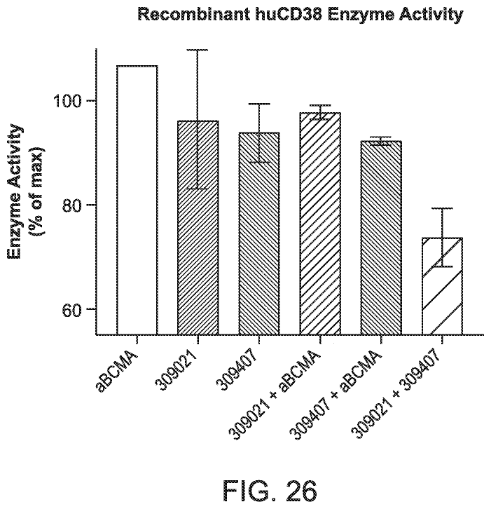

FIG. 26 shows enzyme inhibition of the cyclase activity of CD38 by bivalent and tetravalent UniAbs™. A tetravalent bispecific UniAb™ binding two non-overlapping epitopes on CD38 inhibited cyclase activity potently. Bivalent-monospecific UniAbs™ did not inhibit cyclase activity. An anti-BCMA UniAb™ was used as a negative control. See also FIG. 21, which is a schematic representation of two tetravalent, bispecific heavy chain antibodies and one bivalent bispecific heavy chain antibody.

FIG. 27 shows competition between antibodies for binding to CD38. UniAbs™ from the five sequence families fall into two broad competition groups based on the ability of Daratumamab and Isatuximab to block UniAb binding to CD38+ cells. To identify UniAbs™ with epitopes that partially or completely overlap with epitopes for Daratumumab and Isatuximab, flow cytometry was used to measure percent of UniAb binding that is blocked by pre-treatment of Ramos cells with Daratumumab or Isatuximab. Increasing blocking percentages signal a higher likelihood of the two antibodies having overlapping epitopes. In this set, families F01, F04, F07 and F09 all show at least some level of blocking by both Daratumumab and Isatuximab, indicating likely binding to overlapping epitopes (placing them in competition group 1). In contrast, F03 UniAb (309407) binding is not blocked by pre-treatment with either Daratumumab or Isatuximab, indicating it is likely binding a distinct epitope (placing it in competition group 2).

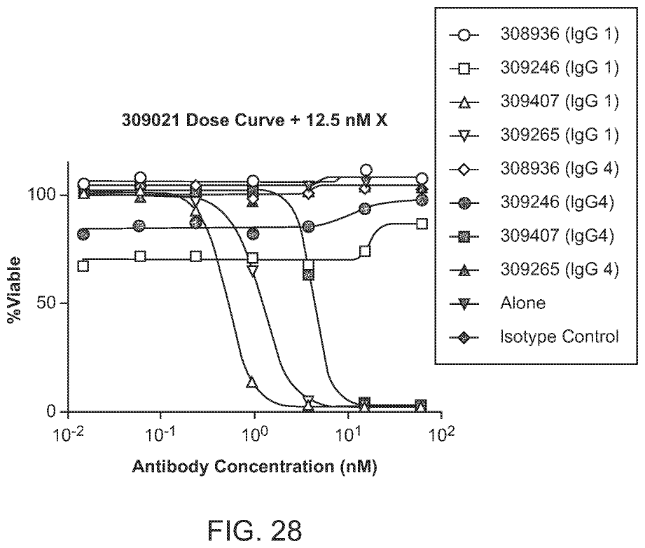

FIG. 28 shows CDC of Ramos cells. UniAb™ 309021 was titrated and mixed with fixed concentration of different UniAbs™ (see legend). UniAbs™ 309407 in IgG1 and IgG4 formats showed synergy with UniAb™ 309021. UniAb™ 309265 in a IgG1 format showed synergy with UniAb 309021™. All other UniAb™ did not synergize with UniAb™ 309021.

### Example 6: CDC-Mediated Cell Death

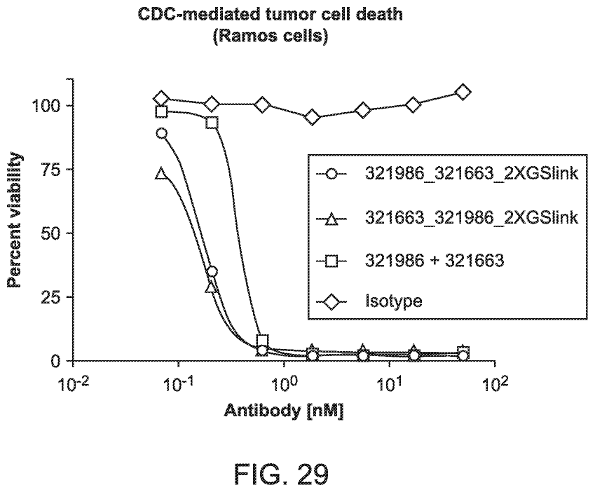

FIG. 29 shows CDC-mediated tumor cell death of Ramos cells by tetravalent bispecific UniAbs™ comprising VH domains of clone ID 321986 and clone ID 321663 compared to a mixture of bivalent monospecific mixture of these same two UniAbs™. These two VH domains bind non-overlapping epitopes on CD38, and combining these VH domains into a single tetravalent antibody (321986_321663-2XGSlink and 321663_321986-2XGSlink) improves killing of tumor cells by CDC compared to a mixture of both bivalent, monospecific UniAbs™ (321986+321663).

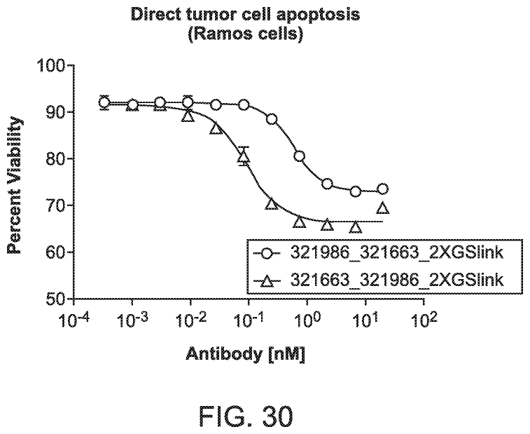

FIG. 30 shows direct tumor cell apoptosis of Ramos cells by tetravalent bispecific UniAbs™ comprising VH domains of clone ID 321986 and clone ID 321663. The efficacy of killing is influenced by the order of the VH domains within the tetravalent molecule. When the VH domain of clone ID 321663 is distal (321663_321986_2XGSlink) (i.e., positioned closer to the N terminus) more potent killing is observed compared to when the VH domain of clone ID 321986 is distal (321986_321663_2XGSlink) (i.e., positioned closer to the C terminus).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising a combination of two or more heavy chain antibodies binding to non-overlapping epitopes on the same ectoenzyme.

2. The composition of claim 1, wherein the ectoenzyme is selected from the group consisting of CD10, CD13, CD26, CD38, CD39, CD73, CD156b, CD156c, CD157, CD203, VAP1, ART2, and MT1-MMP.

3. The composition of claim 2, wherein the ectoenzyme is CD38, CD39 or CD73.

4. The composition of claim 3, wherein the ectoenzyme is CD38.

5. The composition of claim 4, wherein the heavy chain antibody is a UniAb™.

6. The composition of claim 5, wherein the two or more heavy chain antibodies comprise heavy chain variable region amino acid sequences selected from the group consisting of SEQ ID NOs: 1-60, 99-149, 175-218, 247-308, and 323-391.

7. The composition of claim 6, wherein the heavy chain variable region amino acid sequences are selected from the group consisting of SEQ ID NOs: 1, 99, 100 175, 247,323 and 325.

8. The composition of claim 7, wherein the heavy chain variable region amino acid sequences are selected from the group consisting of SEQ ID NOs: 99, 100, 175, 323 and 325.

9. The composition of claim 1, comprising a combination of a first and a second heavy chain antibody, wherein
(a) the first antibody comprises a CDR1 sequence selected from the group consisting of SEQ ID NOs: 150 and 394, a CDR2 sequence selected from the group consisting of SEQ ID NOs: 92 and 413, and a CDR3 sequence selected from the group consisting of SEQ ID NOs: 168 and 431, and
(b) the second antibody comprises a CDR1 sequence selected from the group consisting of SEQ ID NOs: 219 and 393, a CDR2 sequence selected from the group consisting of SEQ ID NOs: 83 and 412, and a CDR3 sequence selected from the group consisting of SEQ ID NOs: 240 and 424.

10. The composition of claim 9, wherein the first antibody comprises a heavy chain variable region amino acid sequence selected from the group consisting of SEQ ID NOs: 100 and 323 and the second antibody comprises a heavy chain variable region amino acid sequence selected from the group consisting of SEQ ID NOs: 175 and 325.

11. The composition of claim 10, wherein the first and the second antibodies are IgG1.

12. The composition of claim 10, wherein the combination is synergistic.

13. The composition of claim 10, comprising a combination of UniAbs™ 309021 and 309265 or a combination of UniAbs™ 321986 and 321663.

14. The composition of claim 1, comprising a combination of a first and a second heavy chain antibody, wherein
(a) the first antibody comprises a CDR1 sequence of SEQ ID NO: 394, a CDR2 sequence of SEQ ID NO: 413, and a CDR3 sequence of SEQ ID NO: 431, and
(b) the second antibody comprises a CDR1 sequence of SEQ ID NO: 151, a CDR2 sequence of SEQ ID NO: 163 and a CDR3 sequence of SEQ ID NO: 172.

15. The composition of claim 14, wherein the first antibody comprises a heavy chain variable region amino acid sequence of SEQ ID NO: 323 and the second antibody comprises a heavy chain variable region amino acid sequence of 99.

16. The composition of claim 15, wherein the first and the second antibodies are IgG1 or IgG4.

17. The composition of claim 15, wherein the combination is synergistic.

18. The composition of claim 15, comprising a combination of UniAbs™ 309021 and 309407.

19. The composition of claim 1, comprising a UniAb™ selected from the group consisting of 309021, 309407 and 309265.

20. A multi-specific heavy chain antibody having binding specificity to at least two non-overlapping epitopes on an ectoenzyme.

21. The multi-specific antibody of claim 20, wherein the ectoenzyme is selected from the group consisting of CD10, CD13, CD26, CD38, CD39, CD73, CD156b, CD156c, CD157, CD203, VAP1, ART2, and MT1-MMP.

22. The multi-specific antibody of claim 21, wherein the ectoenzyme is CD38, CD39 or CD73.

23. The multi-specific antibody of claim 22, wherein the ectoenzyme is CD38.

24. The multi-specific antibody of claim 23, comprising two or more heavy chain variable region amino acid sequences binding to non-ovelapping epitopes on CD38, selected from the group consisting of SEQ ID NOs: 1-60, 99-149, 175-218, 247-308, and 323-391.

25. The multi-specific antibody of any one of claims 21-24, which is bispecific.

26. The multi-specific antibody of claim 25, which is bivalent.

27. The multi-specific antibody of claim 25, which is tetravalent.

28. The multi-specific antibody of claim 22, which is bispecific comprising (a) a first heavy chain variable region comprising a CDR1 sequence selected from the group consisting of SEQ ID NOs: 150 and 394, a CDR2 sequence selected from the group consisting of SEQ ID NOs: 92 and 413, and a CDR3 sequence selected from the group consisting of SEQ ID NOs: 168 and 431, and (b) a second heavy chain variable region comprising a CDR1 sequence selected from the group consisting of SEQ ID NOs: 219 and 393, a CDR2 sequence selected from the group consisting of SEQ ID NOs:
83 and 412 and a CDR3 sequence selected from the group consisting of SEQ ID NO: 240 and 424.

29. The multi-specific antibody of claim 28, comprising a first heavy chain variable region sequence selected from the group consisting of SEQ ID NOs: SEQ ID NO: 100 and 323 and a second heavy chain variable region sequence selected from the group consisting of SEQ ID NOs: 175 and 325.

30. The multi-specific antibody of claim 28 or 29, which is bivalent.

31. The multi-specific antibody of claim 28 or 29, which is tetravalent.

32. The multi-specific antibody of claim 28 or 29, which is IgG1.

33. The multi-specific antibody of claim 22, which is bispecific comprising (a) a first heavy chain variable region comprising a CDR1 sequence of SEQ ID NO: 394, a CDR2 sequence of SEQ ID NO: 413, and a CDR3 sequence of SEQ ID NO: 431, and (b) a second heavy chain variable region comprising a CDR1 sequence of SEQ ID NO: 151, a CDR2 sequence of SEQ ID NO: 163 and a CDR3 sequence of SEQ ID NO: 172.

34. The multi-specific antibody of claim 33, comprising a first heavy chain variable region sequence of SEQ ID NO: SEQ ID NO: 323 and a second heavy chain variable region sequence of SEQ ID NO: 99.

35. The multi-specific antibody of claim 33 or 34, which is bivalent.

36. The multi-specific antibody of claim 33 or 34, which is tetravalent.

37. The multi-specific antibody of claim 33 or 34, which is IgG1 or IgG4.

38. The multi-specific antibody of any one of claims 20-37, which is a UniAb™.

39. A multi-specific antibody, comprising binding specificity of one or more of UniAbs™ 309021, 309265, 309407, 321986, and 321663.

40. The multi-specific antibody of claim 39, comprising binding specificity of UniAbs™ 309021,309265, and 309407.

41. The multi-specific antibody of claim 39, comprising binding specificity of UniAbs™ 321986, and 321663.

42. A CAR-T comprising heavy chain variable region sequences of one or more of multi-specific antibodies of any one or claims 20-41.

43. A pharmaceutical composition comprising a composition of any one or claims 1-19, a multi-specific antibody of any one of claims 20-41 or a CAR-T of claim 42.

44. A method for the treatment of a disease or condition characterized by expression of an ectoenzyme, comprising administering to a subject in need an effective amount of a pharmaceutical composition of claim 43.

45. A method for the treatment of a disease or condition characterized by expression of CD38, CD39, or CD73, comprising administering to a subject in need an effective amount of a multi-specific heavy chain antibody binding to two or more non-overlapping epitopes on CD38, CD39 or CD73.

46. The method of claim 45, wherein said disease or condition is characterized by expression of CD38.

47. The method of claim 46, wherein said disease or condition is selected from the group consisting of hematological malignancies, conditions characterized by airway hyper-responsiveness, and age-related and metabolic dysfunction characterized by nicotinamide adenine dinucleotide (NAD) decline.

48. The method of claim 47, wherein the hematological malignancy is selected from the group comprising multiple myeloma (MM), non-Hodgkin's lymphoma, B-cell chronic lymphocylic leukemia (CLL), B-cell acute lymphoblastic leukemia (ALL), and dT-cell ALL. The CD38 heavy chain antibodies and pharmaceutical compositions of the present invention can also be used to treat asthma and other conditions characterized by airway hyper-responsiveness, and age-related and metabolic dysfunction characterized by nicotinamide adenine dinucleotide (NAD) decline.

49. The method of claim 48, wherein the hematological malignancy is MM.

50. The method of any one of claims 46 to 49, wherein the multi-specific antibody comprises heavy chain CDR1, CDR2 and CDR3 sequences of two or more of antibodies selected from the group consisting of 309021, 309265, 309407, 321986, and 321663.

51. The method of claim 50, wherein the multi-specific antibody comprises heavy chain variable region sequences of two or more of UniAbs™ selected from the group consisting of 309021, 309265, 309407, 321986, and 321663.

52. The method of claim 50, wherein the multi-specific antibody comprises heavy chain CDR1, CDR2 and CDR3 sequences selected from the group consisting of UniAbs™ 309201 and 309265; and 309021 and 309407; and 321986 and 321663.

53. The method of claim 52, wherein the multi-specific antibody comprises heavy chain variable region sequences selected from the group consisting of UniAbs™ 309201 and 309265; and
309021 and 309407; and 321986 and 321663.

54. The method of any one of claims 49 to 53, further comprising administration of one or more further agents for the treatment of MM.

55. The method of claim 54, wherein the agent is selected from the group consisting of daratumumab, isatuximab, elotuzumab, and chemotherapeutic agents effective in the treatment of MM.

56. The method of claim 55, wherein said chemotherapeutic agent is selected from the group consisting of lenalidomide, dexamethasone, and bortezomib.

57. The method of claim 56, wherein the chemotherapeutic agent is lenalidomide and dexamethasone or bortezomib and dexamethasone.
